SEQUENCE LISTING (77 sequences)

SEQ ID NO: 1 - DNA, 798 bp, synthetic construct
  source 1..798: mol_type = other DNA
atggccctgcccgtgaccgctctcctgctgcctctggccctgctcctccatgctgccagacccgacatccagatgacacagacaaccagcagcctgtccgcttccctcggagacagggtgacaatttcctgcagggccagccaggacatcagcaagtacctgaactggtaccagcagaaacccgacggcaccgtcaagctcctgatctaccacaccagcagactgcacagcggagtgccttccaggttcagcggcagcggctccggcaccgattactccctgaccattagcaacttagaacaggaggacattgccacctacttttgtcagcagggcaacaccctcccctacacctttggaggcggaaccaagttagaaatcaccggcggcggcggcagcggaggaggaggcagcggaggcggaggctccgaggtgaaactgcaggagagcggccccggactggtcgcccctagccaatccctctccgtcacctgcaccgtgagcggagtgagcctgcctgactacggagtgagctggatcagacagccccctaggaaaggactggaatggctgggcgtgatttggggcagcgagaccacctattacaacagcgccctgaagtccagactgacaatcatcaaggacaatagcaaaagccaagtgtttctgaagatgaacagcctgcagaccgatgacaccgccatctattattgcgccaagcactactactacggaggaagctacgctatggattattggggccaaggcacaagcgtgaccgtcagcagcgcggccgcc

SEQ ID NO: 2 - DNA, 921 bp, synthetic construct
  source 1..921: mol_type = other DNA
atggccttaccagtgaccgccttgctcctgccgctggccttgctgctccacgccgccaggccgatggaaaaggatacactgttgttgtgggttctgctcctgtgggtgcccggcagcaccggagatattgtgctgacgcagtctcctgcatcactcgccgtgtctctgggccagcgcgctaccatcagctgcagagcctctgaaagtgttgacaattatggaatttctttcatgaattggttccagcagaagcctggccagcccccgaaactcctcatatatgccgcgtctaatcagggctctggggtccctgctagattttctggcagcggctccggcaccgacttcagtctgaatatacatcccatggaagaagacgataccgccatgtacttttgccaacaatctaaggaggtgccttggacgttcggcggcggtacgaagctggaaattaagggcggcgggggaagcggcggggggggatcaggcgggggtggctccggaggcggtggaagtatgggctggagttggatcttccttttccttctttctggtaccgcgggagtgcactctgaggtgcagctccagcagtccggccccgagctcgtcaagcctggggccagtgtcaagatttcctgtaaggcatctggatatacctttacagattacaatatgcattgggtgaaacagtcacatggaaagtcactcgagtggatcggatacatttacccttacaatggaggaaccggatataatcagaagtttaagagcaaggccacactcacggtggacaattcttcatctacagcctacatggatgttcggtctctgacttccgaggatagtgcggtgtattactgcgccaggggacgccccgctatggattactgggggcagggaacctctgtaacagttagctca

SEQ ID NO: 3 - DNA, 1044 bp, synthetic construct
  source 1..1044: mol_type = other DNA
atggccttaccagtgaccgccttgctcctgccgctggccttgctgctccacgccgccaggccggacttcgtgatgactcagtctcctagctccctgaccgtgacagccggcgagaaggtgaccatgtcctgcaaatctagtcagagtctgctgaactcaggcaatcagaagaactatctgacatggtacctgcagaagccagggcagccccctaaactgctgatctattgggccagcaccagggaatccggcgtgcccgacagattcaccggctccgggtctggaacagattttactctgaccatttcaagcgtgcaggccgaggacctggctgtgtactattgtcagaatgattacagctatccctacacatttggcgggggaactaagctggaaatcaaaggtggtggtggttctggtggtggtggttccggcggcggcggctccggtggtggtggatccgaggtgcagctgcagcagagtggacccgaactggtgaaacctggcgcctccgtgaaaatgtcttgcaaggctagtgggtacaccttcacagactactatatgaaatgggtgaagcagtcacacgggaagagcctggagtggatcggagatatcattccctctaacggcgccactttctacaatcagaagtttaaaggcaaggctactctgaccgtggaccggagctcctctaccgcctatatgcacctgaacagtctgacatcagaagatagcgctgtgtactattgtacacggtcccatctgctgagagcctcttggtttgcttattggggccaggggacactggtgactgtgagctccgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtg

SEQ ID NO: 4 - DNA, 252 bp, synthetic construct
  source 1..252: mol_type = other DNA
tccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatct

SEQ ID NO: 5 - DNA, 72 bp, synthetic construct
  source 1..72: mol_type = other DNA
ccattttttttctgctgcttcatcgctgtagccatgggaatccgtttcattattatggtaacaatatggagt

SEQ ID NO: 6 - DNA, 252 bp, synthetic construct
  source 1..252: mol_type = other DNA
cacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaa

SEQ ID NO: 7 - DNA, 73 bp, synthetic construct
  source 1..73: mol_type = other DNA
gtctctttctgcttggtgatggtactcctttttgcagtggacacaggactatatttctctgtgaagacaaaca

SEQ ID NO: 8 - DNA, 252 bp, synthetic construct
  source 1..252: mol_type = other DNA
tgtagaatctaccgcccttctgacaactctgtctctaagtccgtcagattctatctggtggtatctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagcccccattgcc

SEQ ID NO: 9 - DNA, 72 bp, synthetic construct
  source 1..72: mol_type = other DNA
ctggtgcctgtgttctgtggactcctcgtagccaagagcctggtgctgtcagccctgctcgtctggtggggg

SEQ ID NO: 10 - DNA, 252 bp, synthetic construct
  source 1..252: mol_type = other DNA
tccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggg

SEQ ID NO: 11 - DNA, 72 bp, synthetic construct
  source 1..72: mol_type = other DNA
gctggtacagtcctcctccttcgggctggattctatgctgtcagctttctctctgtggccgtgggcagcacc

SEQ ID NO: 12 - DNA, 252 bp, synthetic construct
  source 1..252: mol_type = other DNA
ttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcgg

SEQ ID NO: 13 - DNA, 72 bp, synthetic construct
  source 1..72: mol_type = other DNA
atgggcctggcctttctagtcctggtggctctagtgtggttcctggttgaagactggctcagcaggaagagg

SEQ ID NO: 14 - DNA, 252 bp, synthetic construct
  source 1..252: mol_type = other DNA
agggaaggggaggcccatgaacgtaggctccctgcagtgcgcagcatcaacggaacattccaggccgactttcctctgggccctgccacccacggagggacctacagatgcttcggctctttccgtgacgctccctacgagtggtcaaactcgagtgatccactgcttgtttccgtcacaggaaacccttcaaatagttggccttcacccactgaaccaagctccaaaaccggtaaccccagacacctacat

SEQ ID NO: 15 - DNA, 69 bp, synthetic construct
  source 1..69: mol_type = other DNA
gttctgattgggacctcagtggtcaaaatccctttcaccatcctcctcttctttctccttcatcgctgg

SEQ ID NO: 16 - DNA, 210 bp, synthetic construct
  source 1..210: mol_type = other DNA
atgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaag

SEQ ID NO: 17 - DNA, 78 bp, synthetic construct
  source 1..78: mol_type = other DNA
ctcactgccgaggtcctaggaatcatttgcattgtcctgatggccactgtgttaaaaacaatagttcttattcctttc

SEQ ID NO: 18 - DNA, 252 bp, synthetic construct
  source 1..252: mol_type = other DNA
gtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 19 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 20 - DNA, 252 bp, synthetic construct
  source 1..252: mol_type = other DNA
gcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacacc

SEQ ID NO: 21 - DNA, 84 bp, synthetic construct
  source 1..84: mol_type = other DNA
attccgtggctcggccacctcctcgtgggcctcagcggggcttttggcttcatcatcttagtgtacttgctgatcaactgcagg

SEQ ID NO: 22 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 23 - DNA, 182 bp, synthetic construct
  source 1..182: mol_type = other DNA
aaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttaag

SEQ ID NO: 24 - DNA, 512 bp, synthetic construct
  source 1..512: mol_type = other DNA
tttctcagggagtattgctaaaatgccaaaaaggaagttgttcaaatgccactcagtatgaggacactggagatctaaaagtgaataatggcacaagaagaaatataagtaataaggacctttgtgcttcgagatctgcagaccagacagtactatgccaatcagaatggctcaaataccaagggaagtgttattggttctctaatgagatgaaaagctggagtgacagttatgtgtattgtttggaaagaaaatctcatctactaatcatacatgaccaacttgaaatggcttttatacagaaaaacctaagacaattaaactacgtatggattgggcttaactttacctccttgaaaatgacatggacttgggtggatggttctccaatagattcaaagatattcttcataaagggaccagctaaagaaaacagctgtgctgccattaaggaaagcaaaattttctctgaaacctgcagcagtgttttcaaatggatttgtcagtattag

SEQ ID NO: 25 - DNA, 251 bp, synthetic construct
  source 1..251: mol_type = other DNA
agaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtg

SEQ ID NO: 26 - DNA, 242 bp, synthetic construct
  source 1..242: mol_type = other DNA
attccctatctttgtctactcagcgaacacagggccccgcagagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtgtaa

SEQ ID NO: 27 - DNA, 167 bp, synthetic construct
  source 1..167: mol_type = other DNA
agtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaag

SEQ ID NO: 28 - DNA, 351 bp, synthetic construct
  source 1..351: mol_type = other DNA
aaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaa

SEQ ID NO: 29 - DNA, 210 bp, synthetic construct
  source 1..210: mol_type = other DNA
atccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 30 - DNA, 144 bp, synthetic construct
  source 1..144: mol_type = other DNA
aagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatga

SEQ ID NO: 31 - DNA, 141 bp, synthetic construct
  source 1..141: mol_type = other DNA
aaggctctgatccacctgagcgacctccgggagtacaggcgctttgagaaggagaagctcaagtcccagtggaacaatgataatccccttttcaagagcgccaccacgacggtcatgaaccccaagtttgctgagagttag

SEQ ID NO: 32 - DNA, 144 bp, synthetic construct
  source 1..144: mol_type = other DNA
aaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaa

SEQ ID NO: 33 - DNA, 861 bp, synthetic construct
  source 1..861: mol_type = other DNA
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 34 - DNA, 573 bp, synthetic construct
  source 1..573: mol_type = other DNA
tataaagttgacttggttctgttctataggcgcatagcggaaagagacgagacactaacagatggtaaaacatatgatgcctttgtgtcttacctgaaagagtgtcatcctgagaataaagaagagtatacttttgctgtggagacgttacccagggtcctggagaaacagtttgggtataagttatgcatatttgaaagagatgtggtgcctggcggagctgttgtcgaggagatccattcactgatagagaaaagccggaggctaatcatcgttctcagccagagttacctgactaacggagccaggcgtgagctcgagagtggactccacgaagcactggtagagaggaagattaagatcatcttaattgagtttactccagccagcaacatcacctttctccccccgtcgctgaaactcctgaagtcctacagagttctaaaatggagggctgacagtccctccatgaactcaaggttctggaagaatcttgtttacctgatgcccgcaaaagccgtcaagccatggagagaggagtcggaggcgcggtctgttctctcagcaccttga

SEQ ID NO: 35 - DNA, 279 bp, synthetic construct
  source 1..279: mol_type = other DNA
aacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtga

SEQ ID NO: 36 - DNA, 660 bp, synthetic construct
  source 1..660: mol_type = other DNA
cattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 37 - DNA, 858 bp, synthetic construct
  source 1..858: mol_type = other DNA
agcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 38 - DNA, 1542 bp, synthetic construct
  source 1..1542: mol_type = other DNA
cccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctctga

SEQ ID NO: 39 - RNA, 20 nt, synthetic construct
  source 1..20: mol_type = other RNA
gcggctgtggtggtgccttc

SEQ ID NO: 40 - RNA, 20 nt, synthetic construct
  source 1..20: mol_type = other RNA
gcagctgtggtggtgccttc

SEQ ID NO: 41 - RNA, 20 nt, synthetic construct
  source 1..20: mol_type = other RNA
ccagaagtgggcggctgtgg

SEQ ID NO: 42 - RNA, 20 nt, synthetic construct
  source 1..20: mol_type = other RNA
agcaggaggggccggagtat

SEQ ID NO: 43 - DNA, 20 bp, synthetic construct
  source 1..20: mol_type = other DNA
gcaggacgcctacgacggca

SEQ ID NO: 44 - DNA, 639 bp, synthetic construct
  source 1..639: mol_type = other DNA
atggccttaccagtgaccgccttgctcctgccgctggccttgctgctccacgccgccaggccgggcatgcgcaccgaggacctgcccaaggccgtggtgttcctggagccccagtggtaccgcgtgctggagaaggactccgtgaccctgaagtgccagggcgcctactcccccgaggacaactccacccagtggttccacaacgagtccctgatctcctcccaggcctcctcctacttcatcgacgccgccaccgtggacgactccggcgagtaccgctgccagaccaacctgtccaccctgtccgaccccgtgcagctggaggtgcacatcggctggctgctgctgcaggccccccgctgggtgttcaaggaggaggaccccatccacctgcgctgccactcctggaagaacaccgccctgcacaaggtgacctacctgcagaacggcaagggccgcaagtacttccaccacaactccgacttctacatccccaaggccaccctgaaggactccggctcctacttctgccgcggcctgttcggctccaagaacgtgtcctccgagaccgtgaacatcaccatcacccagggcctggccgtgcccaccatctcctccttcttcccccccggctaccag

SEQ ID NO: 45 - DNA, 831 bp, synthetic construct
  source 1..831: mol_type = other DNA
caggtggacaccaccaaggccgtgatcaccctgcagcccccctgggtgtccgtgttccaggaggagaccgtgaccctgcactgcgaggtgctgcacctgcccggctcctcctccacccagtggttcctgaacggcaccgccacccagacctccaccccctcctaccgcatcacctccgcctccgtgaacgactccggcgagtaccgctgccagcgcggcctgtccggccgctccgaccccatccagctggagatccaccgcggctggctgctgctgcaggtgtcctcccgcgtgttcaccgagggcgagcccctggccctgcgctgccacgcctggaaggacaagctggtgtacaacgtgctgtactaccgcaacggcaaggccttcaagttcttccactggaactccaacctgaccatcctgaagaccaacatctcccacaacggcacctaccactgctccggcatgggcaagcaccgctacacctccgccggcatctccgtgaccgtgaaggagctgttccccgcccccgtgctgaacgcctccgtgacctcccccctgctggagggcaacctggtgaccctgtcctgcgagaccaagctgctgctgcagcgccccggcctgcagctgtacttctccttctacatgggctccaagaccctgcgcggccgcaacacctcctccgagtaccagatcctgaccgcccgccgcgaggactccggcctgtactggtgcgaggccgccaccgaggacggcaacgtgctgaagcgctcccccgagctggagctgcaggtgctgggcctgcagctgcccacccccgtgtggttccac

SEQ ID NO: 46 - DNA, 621 bp, synthetic construct
  source 1..621: mol_type = other DNA
atgcagtccggcacccactggcgcgtgctgggcctgtgcctgctgtccgtgggcgtgtggggccaggacggcaacgaggagatgggcggcatcacccagaccccctacaaggtgtccatctccggcaccaccgtgatcctgacctgcccccagtaccccggctccgagatcctgtggcagcacaacgacaagaacatcggcggcgacgaggacgacaagaacatcggctccgacgaggaccacctgtccctgaaggagttctccgagctggagcagtccggctactacgtgtgctacccccgcggctccaagcccgaggacgccaacttctacctgtacctgcgcgcccgcgtgtgcgagaactgcatggagatggacgtgatgtccgtggccaccatcgtgatcgtggacatctgcatcaccggcggcctgctgctgctggtgtactactggtccaagaaccgcaaggccaaggccaagcccgtgacccgcggcgccggcgccggcggccgccagcgcggccagaacaaggagcgccccccccccgtgcccaaccccgactacgagcccatccgcaagggccagcgcgacctgtactccggcctgaaccagcgccgcatc

SEQ ID NO: 47 - DNA, 546 bp, synthetic construct
  source 1..546: mol_type = other DNA
atggagcagggcaagggcctggccgtgctgatcctggccatcatcctgctgcagggcaccctggcccagtccatcaagggcaaccacctggtgaaggtgtacgactaccaggaggacggctccgtgctgctgacctgcgacgccgaggccaagaacatcacctggttcaaggacggcaagatgatcggcttcctgaccgaggacaagaagaagtggaacctgggctccaacgccaaggacccccgcggcatgtaccagtgcaagggctcccagaacaagtccaagcccctgcaggtgtactaccgcatgtgccagaactgcatcgagctgaacgccgccaccatctccggcttcctgttcgccgagatcgtgtccatcttcgtgctggccgtgggcgtgtacttcatcgccggccaggacggcgtgcgccagtcccgcgcctccgacaagcagaccctgctgcccaacgaccagctgtaccagcccctgaaggaccgcgaggacgaccagtactcccacctgcagggcaaccagctgcgccgcaac

SEQ ID NO: 48 - DNA, 183 bp, synthetic construct
  source 1..183: mol_type = other DNA
cgcaagcgctggcagaacgagaagctgggcctggacgccggcgacgagtacgaggacgagaacctgtacgagggcctgaacctggacgactgctccatgtacgaggacatctcccgcggcctgcagggcacctaccaggacgtgggctccctgaacatcggcgacgtgcagctggagaagccc

SEQ ID NO: 49 - DNA, 147 bp, synthetic construct
  source 1..147: mol_type = other DNA
ctggacaaggacgactccaaggccggcatggaggaggaccacacctacgagggcctggacatcgaccagaccgccacctacgaggacatcgtgaccctgcgcaccggcgaggtgaagtggtccgtgggcgagcaccccggccaggag

SEQ ID NO: 50 - RNA, 20 nt, synthetic construct
  source 1..20: mol_type = other RNA
taccggcaggacgcctacga

SEQ ID NO: 51 - RNA, 20 nt, synthetic construct
  source 1..20: mol_type = other RNA
ggagcagcggagagtctacc

SEQ ID NO: 52 - RNA, 20 nt, synthetic construct
  source 1..20: mol_type = other RNA
cgctgcagcgcacgggtacc

SEQ ID NO: 53 - RNA, 20 nt, synthetic construct
  source 1..20: mol_type = other RNA
gacctggcagcgggatgggg

SEQ ID NO: 54 - RNA, 20 nt, synthetic construct
  source 1..20: mol_type = other RNA
ggaggaccagacccaggaca

SEQ ID NO: 55 - RNA, 20 nt, synthetic construct
  source 1..20: mol_type = other RNA
gacccaggacacggagctcg

SEQ ID NO: 56 - RNA, 20 nt, synthetic construct
  source 1..20: mol_type = other RNA
cacaccatccagataatgta

SEQ ID NO: 57 - RNA, 20 nt, synthetic construct
  source 1..20: mol_type = other RNA
ccttaccccatctcagggtg

SEQ ID NO: 58 - RNA, 20 nt, synthetic construct
  source 1..20: mol_type = other RNA
cgagccagaagatggagccg

SEQ ID NO: 59 - RNA, 20 nt, synthetic construct
  source 1..20: mol_type = other RNA
ttaccccatctcagggtgag

SEQ ID NO: 60 - RNA, 20 nt, synthetic construct
  source 1..20: mol_type = other RNA
tgacggccatcctcggcgtc

SEQ ID NO: 61 - RNA, 20 nt, synthetic construct
  source 1..20: mol_type = other RNA
ctacgtagggtccttcatcc

SEQ ID NO: 62 - RNA, 100 nt, synthetic construct
  source 1..100: mol_type = other RNA
  modified_base 1..4: mod_base = OTHER; note = Phosphorothioate linkage
  modified_base 97..100: mod_base = OTHER; note = Phosphorothioate linkages
  modified_base 1: mod_base = gm
  modified_base 2: mod_base = cm
  modified_base 3: mod_base = gm
  modified_base 97..99: mod_base = um
gcggctgtggtggtgccttcgttttagagctagaaatagcaagttaaaataaggctagtccgttatcaacttgaaaaagtggcaccgagtcggtgctttt

SEQ ID NO: 63 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1: mod_base = gm
  modified_base 2: mod_base = cm
  modified_base 3: mod_base = OTHER; note = 2-O-Methyladenosine
  modified_base 97..99: mod_base = um
gcagctgtggtggtgccttcgttttagagctagaaatagcaagttaaaataaggctagtccgttatcaacttgaaaaagtggcaccgagtcggtgctttt

SEQ ID NO: 64 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1..2: mod_base = cm
  modified_base 3: mod_base = OTHER; note = 2-O-methyladenosine
  modified_base 97..99: mod_base = um
ccagaagtgggcggctgtgggttttagagctagaaatagcaagttaaaataaggctagtccgttatcaacttgaaaaagtggcaccgagtcggtgctttt

SEQ ID NO: 65 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1: mod_base = OTHER; note = 2-O-Methyladenosine
  modified_base 2: mod_base = gm
  modified_base 3: mod_base = cm
  modified_base 97..99: mod_base = um
agcaggaggggccggagtatgttttagagctagaaatagcaagttaaaataaggctagtccgttatcaacttgaaaaagtggcaccgagtcggtgctttt

SEQ ID NO: 66 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1: mod_base = gm
  modified_base 2: mod_base = cm
  modified_base 3: mod_base = OTHER; note = 2-O-Methyladenosine
  modified_base 97..99: mod_base = um
gcaggacgcctacgacggcagttttagagctagaaatagcaagttaaaataaggctagtccgttatcaacttgaaaaagtggcaccgagtcggtgctttt

SEQ ID NO: 67 - RNA, 121 nt, synthetic construct
  source 1..121: mol_type = other RNA
  modified_base 1..4: mod_base = OTHER; note = Phosphorothioate linkages
  modified_base 118..121: mod_base = OTHER; note = Phosphorothioate linkages
  modified_base 1..2: mod_base = gm
  modified_base 3: mod_base = OTHER; note = 2-O-Methyladenosine
  modified_base 119: mod_base = cm
  modified_base 120: mod_base = um
  modified_base 121: mod_base = cm
ggatggggaggaccagacccgttttagagctagaaatagcaagttaaaataaggctagtccgttatcaacttgaaaaagtggcaccgagtcggtgcgctccgccttctgggtctggtcctc

SEQ ID NO: 68 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1: mod_base = um
  modified_base 2: mod_base = OTHER; note = 2-O-Methyladenosine
  modified_base 3: mod_base = cm
  modified_base 97..99: mod_base = um
taccggcaggacgcctacgagttttagagctagaaatagcaagttaaaataaggctagtccgttatcaacttgaaaaagtggcaccgagtcggtgctttt

SEQ ID NO: 69 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1..2: mod_base = gm
  modified_base 3: mod_base = OTHER; note = 2-O-Methyladenosine
  modified_base 97..99: mod_base = um
ggagcagcggagagtctaccgttttagagctagaaatagcaagttaaaataaggctagtccgttatcaacttgaaaaagtggcaccgagtcggtgctttt

SEQ ID NO: 70 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1: mod_base = cm
  modified_base 2: mod_base = gm
  modified_base 3: mod_base = cm
  modified_base 97..99: mod_base = um
cgctgcagcgcacgggtaccgttttagagctagaaatagcaagttaaaataaggctagtccgttatcaacttgaaaaagtggcaccgagtcggtgctttt

SEQ ID NO: 71 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1: mod_base = gm
  modified_base 2: mod_base = OTHER; note = 2-O-Methyladenosine
  modified_base 3: mod_base = cm
  modified_base 97..99: mod_base = um
gacctggcagcgggatgggggttttagagctagaaatagcaagttaaaataaggctagtccgttatcaacttgaaaaagtggcaccgagtcggtgctttt

SEQ ID NO: 72 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1..2: mod_base = cm
  modified_base 3: mod_base = um
  modified_base 97..99: mod_base = um
ccttaccccatctcagggtggttttagagctagaaatagcaagttaaaataaggctagtccgttatcaacttgaaaaagtggcaccgagtcggtgctttt

SEQ ID NO: 73 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1: mod_base = cm
  modified_base 2: mod_base = gm
  modified_base 3: mod_base = OTHER; note = 2-O-Methyladenosine
  modified_base 97..99: mod_base = um
cgagccagaagatggagccggttttagagctagaaatagcaagttaaaataaggctagtccgttatcaacttgaaaaagtggcaccgagtcggtgctttt

SEQ ID NO: 74 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1..2: mod_base = um
  modified_base 3: mod_base = OTHER; note = 2-O-Methyladenosine
  modified_base 97..99: mod_base = um
ttaccccatctcagggtgaggttttagagctagaaatagcaagttaaaataaggctagtccgttatcaacttgaaaaagtggcaccgagtcggtgctttt

SEQ ID NO: 75 - RNA, 100 nt, synthetic construct
  source 1..100: mol_type = other RNA
  modified_base 1..4: mod_base = OTHER; note = Phosphorothioate linkages
  modified_base 97..99: mod_base = OTHER; note = Phosphorothioate linkages
  modified_base 1: mod_base = um
  modified_base 2: mod_base = gm
  modified_base 3: mod_base = OTHER; note = 2-O-Methyladenosine
  modified_base 97..99: mod_base = um
tgacggccatcctcggcgtcgttttagagctagaaatagcaagttaaaataaggctagtccgttatcaacttgaaaaagtggcaccgagtcggtgctttt

SEQ ID NO: 76 - RNA, 100 nt, synthetic construct
  source 1..100: mol_type = other RNA
  modified_base 1..4: mod_base = OTHER; note = Phosphorothioate linkages
  modified_base 97..100: mod_base = OTHER; note = Phosphorothioate linkages
  modified_base 1: mod_base = cm
  modified_base 2: mod_base = um
  modified_base 3: mod_base = OTHER; note = 2-O-Methyladenosine
  modified_base 97..99: mod_base = um
ctacgtagggtccttcatccgttttagagctagaaatagcaagttaaaataaggctagtccgttatcaacttgaaaaagtggcaccgagtcggtgctttt

SEQ ID NO: 77 - RNA, 20 nt, synthetic construct
  source 1..20: mol_type = other RNA
ccagaagtgggcggctgtgg

(19) United States
(12) Patent Application Publication
(10) Pub. No.: US 2024/0189355 A1
(43) Pub. Date: Jun. 13, 2024

(54) HYPOIMMUNOGENIC CELLS HAVING TARGETED MODIFICATIONS IN MHC CLASS-I GENES AND METHODS OF USE

(71) Applicant: Wugen, Inc., Saint Louis, MO (US)

(72) Inventors: You ZHOU, Creve Coeur, MO (US); Matthew COOPER, St. Louis, MO (US); Elizabeth SCHRAMM, O'Fallon, IL (US)

(21) Appl. No.: 18/509,110

(22) Filed: Nov. 14, 2023

Related U.S. Application Data
(60) Provisional application No. 63/425,272, filed on Nov. 14, 2022.

(51) Int. Cl. A61K 35/17; A61K 39/00; A61P 35/00; C07K 14/74; C12N 5/0783; C12N 9/22

(52) U.S. Cl. CPC A61K 35/17; A61K 39/4611; A61K 39/4613; A61K 39/4631; A61K 39/464411; A61P 35/00; C07K 14/70539; C12N 5/0636; C12N 5/0646; C12N 9/22; A61K 2239/15; A61K 2239/30; C12N 2310/20

(57) ABSTRACT

The present disclosure provides compositions of genetically modified cells that are hypoimmunogenic. The present disclosure also provides methods of making hypoimmunogenic cells and using hypoimmunogenic cells for the treatment of cancer and autoimmune conditions.

### CROSS REFERENCE TO RELATED APPLICATIONS

This application claims priority to U.S. Provisional Application Ser. No. 63/425,272, filed on Nov. 14, 2022, which is incorporated by reference herein in its entirety for all purposes.

### SEQUENCE LISTING

The Sequence Listing XML associated with this application is provided in XML file format and is hereby incorporated by reference into the specification. The name of the XML file containing the Sequence Listing XML is WUGE_003_01US_ST26.xml. The XML file is 124,465 bytes in size, was created on Nov. 14, 2023, and is being submitted electronically via USTPO Patent Center.

### FIELD

The present disclosure pertains to hypoimmunogenic cells and compositions thereof, as well as methods of making or using such hypoimmunogenic cells and compositions.

### BACKGROUND

Allogeneic cell therapies targeting cancer and other diseases have many benefits over autologous cell therapies. For example, they are off-the-shelf compatible, which utilizes an economy of scale to make products in a more efficient and cost-effective manner. Importantly, this reduces the lag time before a patient can be treated. In addition, allogeneic cell therapies are derived from healthy donors, which reduces the risk of manufacturing problems.

Despite these and other advantages, allogeneic cell products must overcome alloreactivity of a recipient patient's immune system in order to be effective. Briefly, mammalian somatic cells express a poly-allelic and highly polymorphic protein on cell surfaces known as the Major Histocompatibility Complex class I (MHC-I), which is encoded by the Human Leukocyte Antigen (HLA) gene. Most individuals express twelve heterogeneous variants of the MHC-I protein. When an allogeneic cell is transplanted into a patient, mismatched MHC-I proteins are recognized by the patient's immune system as foreign. A resulting CD8 cytotoxic T cell response is mounted, and immune rejection of the transplanted cells occurs. This is the primary reason that many solid tissue transplants are rejected.

To address this issue, various conditioning regimens have been used in order to deplete a patient's T cells prior to the injection of allogeneic cellular therapies. However, these conditioning regimens are associated with several drawbacks, including exposing the patient to a significantly increased infection risk. In addition, it's becoming evident that a patient's own T cells are important for achieving robust and durable control of cancer in the patient. Accordingly, alternative approaches attempting to “mask” the allogeneic cell therapies from CD8+ T cells have been explored. These efforts have primarily utilized the abrogation of MHC expression on the cell surface via genetic manipulations, including deletion of MHC chaperone proteins, such as beta-2-microglobulin (B2M) or TAP1/2 on the allogeneic cells.

While these targeted approaches spare a patient's immune system, they provide only an incomplete protection of the allogeneic cell therapy from rejection by the patient's immune system. This is due primarily to what is known as the “missing self” response, whereby a patient's NK cells will seek and eliminate cells missing surface expression of MHC. As a result, the deletion of MHC from the cell surface to avoid a T cell response induces an unwanted NK response against the cells. Therefore, additional steps are required in conjunction with MHC deletion in order to prevent an NK cell “missing self” response. These steps have included, for example, the re-expression of HLA-E, which is a less polymorphic MHC-I molecule, along with an exogenous copy of B2M and peptide as a “triple chain.” However, these and other alternative approaches are complicated and pose significant technical challenges.

Accordingly, there remains an important unmet need for compositions and methods that effectively abrogate immune rejection of allogenic cell therapies. As further described herein, the present disclosure meets this need and offers other related advantages.

### SUMMARY

The present disclosure provides a hypoimmunogenic cell comprising a genetic modification in one or more of HLA-A, HLA-B and HLA-C alleles, where the modification (i) reduces or prevents a CD8+ T cell response against the cell, and (ii) reduces or prevents induction of an NK cell missing self response against the cell.

In certain embodiments of the present disclosure, a genetic modification in the cell is in the alpha3 domain of the HLA-A, HLA-B and HLA-C alleles. In more particular embodiments, the genetic modification in the alpha3 domain comprises a mutation in the HLA-A, HLA-B and HLA-C alleles selected from the group consisting of A245V, D227K, T228A, K66A and R65A. In some embodiments, a combination of the one or more genetic modificationss is introduced. In other more particular embodiments, the genetic modification in the alpha3 domain further comprises a mutation in the HLA-E, HLA-F and/or HLA-G alleles.

In certain other embodiments of the present disclosure, a genetic modification in cell comprises one or more introduced in-frame stop codon mutations or other mutations for disrupting expression (e.g., by base editing splice junctions, start codons, promoter sequence motifs and the like). In more particular embodiments, the in-frame stop codon mutation is in the HLA-A, HLA-B and HLA-C alleles. In more particular embodiments, the introduced in-frame stop codon mutation in the cell is in the alpha1, alpha2 or alpha3 domain of the HLA-A, HLA-B and HLA-C alleles. In some more particular embodiments, where an in-frame stop codon is introduced, the cell further comprises a heterologous transgene encoding HLA-E.

In some embodiments, the genetic modification introduced in a cell according to the present disclosure is introduced using a gene editing technique. For example, in some embodiments, the genetic modification is introduced in the cell using a gene editing technique, such as prime editing and base editing.

In some embodiments, the type of cell used according to the present disclosure is a human cell. In other embodiments, the cell is an immune cell. In still other embodiments, the cell is an immune cell selected from a T cell, a B cell, an NK cell and a dendritic cell. In other embodiments, the cell is an induced pluripotent stem cell (iPSC). In still other embodiments, the cell is a somatic cell differentiated or derived from an iPSC.

In additional embodiments, a hypoimmunogenic cell of the present disclosure further comprises at least one heterologous transgene, such as a heterologous transgene encoding a chimeric antigen receptor.

According to another aspect of the present disclosure, there is provided a method of preparing a hypoimmunogenic cell, the method comprising a step of introducing into a cell a genetic modification in the HLA-A, HLA-B and HLA-C alleles, where the modification (i) reduces or prevents a CD8+ T cell response against the cell, and (ii) reduces or prevents induction of an NK cell missing self response against the cell.

In some embodiments, the genetic modification in a cell is introduced is in the alpha3 domain of the HLA-A, HLA-B and HLA-C alleles. In more particular embodiments, the genetic modification in the alpha3 domain comprises a mutation in the HLA-A, HLA-B and HLA-C alleles selected from the group consisting of A245V, D227K, T228A, K66A and R65A. In some embodiments, a combination of the one or more genetic modificationss is introduced. In other more particular embodiments, the genetic modification in the alpha3 domain further comprises a mutation in the HLA-E, HLA-F and/or HLA-G alleles.

In some embodiments, the genetic modification introduced in the method comprises an introduced in-frame stop codon mutation. For example, in more particular embodiments, the introduced in-frame stop codon mutation is in the alpha1, alpha2 or alpha3 domain of the HLA-A, HLA-B and HLA-C alleles. In some more particular embodiments, where an in-frame stop codon is introduced within the genome of a cell, the cell further comprises a heterologous transgene encoding HLA-E.

The genetic modification introduced in a cell of the present disclosure can be carried out using any available technique. In some embodiments, the genetic modification in the cell is introduced using a gene editing technique, such as prime editing or base editing.

In certain more particular embodiments, a genetic modification introduced in the cell is an A245V mutation introduced using a small guide RNA comprising a sequence selected from the group consisting of GCGGCUGUGGUGGUGCCUUC (SEQ ID NO: 39) and GCAGCUGUGGUGGUGCCUUC (SEQ ID NO: 40).

In other more particular embodiments, a genetic modification introduced in a cell is an in-frame stop codon mutation introduced using a small guide RNA comprising a sequence selected from the group consisting of CCAGAAGUGGGCGGCUGUGG (SEQ ID NO: 41), AGCAGGAGGGGCCGGAGUAU (SEQ ID NO: 42) and GCAGGACGCCUACGACGGCA (SEQ ID NO: 43). In some embodiments, where an in-frame stop codon is introduced, the method further comprises a step of introducing a heterologous transgene encoding HLA-E.

A cell prepared according to the disclosed methods can be essentially any desired cell type. For example, in some embodiments, the cell is a human cell, such as an immune cells, such as, but not limited to, a T cell, a B cell, an NK cell and a dendritic cell. In other embodiments, the cell is a stem cell, such as an induced pluripotent stem cell (iPSC). In still other embodiments, the cell is a somatic cell differentiated or derived from an iPSC.

In some cases, in the methods of the disclosure, there further comprises a step of introducing at least one heterologous transgene, such as a heterologous transgene encoding a chimeric antigen receptor.

According to yet another aspect, the present disclosure provides a pharmaceutical composition comprising (i) hypoimmunogenic cells as described herein or (ii) hypoimmunogenic cells prepared according to the methods as described herein, in combination with a physiologically acceptable excipient.

According to still another aspect, the present disclosure provides a method of treating a disease or condition comprising administering to a subject in need thereof a pharmaceutical composition as described herein. In particular embodiments, the disease or condition is selected from the group consisting of cancer, myocardial infarction, blindness, spinal cord injury, ALS, Parkinson's disease, Huntington's disease, Alzheimer's disease, enteric neuropathy, multiple sclerosis, osteoarthritis, skin disease, diabetes, liver disease, osteoporosis, DiGeorge syndrome, kidney disease and an immune disorder.

The disclosure provides an hypoimmunogenic cell comprising one or more genetic modifications in one or more HLA genes selected from the group consisting of: HLA-A, HLA-B, HLA-C, HLA-E, HLA-F, and HLA-G, wherein the modification (i) reduces or prevents a CD8+ T cell-mediated response against the hypoimmunogenic cell, and/or (ii) reduces or prevents an NK cell-mediated response against the hypoimmunogenic cell.

In some embodiments of the hypoimmunogenic cell of the disclosure, the one or more genetic modifications comprises a modification in one or more domains of the one or more HLA genes selected from the group consisting of: alpha 1 domain, alpha 2 domain, alpha 3 domain, transmembrane domain, intracellular domain, and signal peptide region.

In some embodiments of the hypoimmunogenic cell of the disclosure, the one or more genetic modifications comprises a modification in the alpha 3 domain of the one or more HLA genes.

In some embodiments of the hypoimmunogenic cell of the disclosure, the one or more genetic modifications encode one or more single amino acid substitutions.

In some embodiments of the hypoimmunogenic cell of the disclosure, the one or more genetic modifications encode one or more modifications at one or more amino acid residues selected from the group consisting of: A245, D227, T228, K66, and R65.

In some embodiments of the hypoimmunogenic cell of the disclosure, the one or more genetic modifications encode one or more amino acid substitutions selected from the group consisting of: A245V, D227K, T228A, K66A, and R65A.

In some embodiments of the hypoimmunogenic cell of the disclosure, the one or more genetic modifications encode a modification at A245 in HLA-A, HLA-B, and HLA-C.

In some embodiments of the hypoimmunogenic cell of the disclosure, the modification is A245V.

In some embodiments of the hypoimmunogenic cell of the disclosure, the one or more genetic modifications encode a modification at D227 in HLA-A, HLA-B, and HLA-C and a modification at T228 in HLA-A, HLA-B, and HLA-C.

In some embodiments of the hypoimmunogenic cell of the disclosure, the modification at D227 is D227K and the modification at T228 is T228A.

In some embodiments of the hypoimmunogenic cell of the disclosure, the one or more genetic modifications comprises one or more in-frame stop codon mutations.

In some embodiments of the hypoimmunogenic cell of the disclosure, the hypoimmunogenic cell comprises one or more exogenously expressed HLA genes, optionally wherein the HLA genes comprise one or more mutations.

In some embodiments of the hypoimmunogenic cell of the disclosure, the hypoimmunogenic cell comprises an exogenously expressed HLA-E gene.

In some embodiments of the hypoimmunogenic cell of the disclosure, the genetic modification is introduced using a gene editing technique, optionally wherein the gene editing technique is selected from the group consisting of CRISPR-Cas9 gene editing, prime editing, and base editing.

In some embodiments of the hypoimmunogenic cell of the disclosure, the hypoimmunogenic cell is differentiated or derived from a stem cell, optionally wherein the stem cell is an induced pluripotent stem cell (iPSC).

In some embodiments of the hypoimmunogenic cell of the disclosure, the hypoimmunogenic cell is an immune cell.

In some embodiments of the hypoimmunogenic cell of the disclosure, the hypoimmunogenic cell is selected from the group consisting of: a T cell, a B cell, an NK cell, a dendritic cell, and a macrophage.

In some embodiments of the hypoimmunogenic cell of the disclosure, the hypoimmunogenic cell further comprises a polynucleotide encoding a cell surface receptor, optionally wherein the cell surface receptor is a chimeric receptor.

The disclosure provides a method of making a hypoimmunogenic cell, the method comprising introducing into a cell one or more genetic modifications in one or more HLA genes selected from the group consisting of: HLA-A, HLA-B, HLA-C, HLA-E, HLA-F, and HLA-G; wherein the modification (i) reduces or prevents a CD8+ T cell-mediated response against the hypoimmunogenic cell, and/or (ii) reduces or prevents an NK cell-mediated response against the hypoimmunogenic cell.

In some embodiments of the method of making a hypoimmunogenic cell of the disclosure, the one or more genetic modifications comprises a modification in one or more domains of the one or more HLA genes selected from the group consisting of: alpha 1 domain, alpha 2 domain, alpha 3 domain, transmembrane domain, intracellular domain, and signal peptide region.

In some embodiments of the method of making a hypoimmunogenic cell of the disclosure, the one or more genetic modifications comprises a modification in the alpha 3 domain of the one or more HLA genes.

In some embodiments of the method of making a hypoimmunogenic cell of the disclosure, the one or more genetic modifications encode one or more single amino acid substitutions.

In some embodiments of the method of making a hypoimmunogenic cell of the disclosure, the one or more genetic modifications encode one or more modifications at one or more amino acid residues selected from the group consisting of: A245, D227, T228, K66, and R65.

In some embodiments of the method of making a hypoimmunogenic cell of the disclosure, the one or more genetic modifications encode one or more amino acid substitutions selected from the group consisting of: A245V, D227K, T228A, K66A, and R65A.

In some embodiments of the method of making a hypoimmunogenic cell of the disclosure, the one or more genetic modifications encode a modification at A245 in HLA-A, HLA-B, and HLA-C.

In some embodiments of the method of making a hypoimmunogenic cell of the disclosure, the modification is A245V.

In some embodiments of the method of making a hypoimmunogenic cell of the disclosure, the one or more genetic modifications encode an A245V mutation introduced using a small guide RNA comprising the sequence GCGGCUGUGGUGGUGCCUUC (SEQ ID NO: 39) or GCAGCUGUGGUGGUGCCUUC (SEQ ID NO: 40).

In some embodiments of the method of making a hypoimmunogenic cell of the disclosure, the one or more genetic modifications encode a modification at D227 in HLA-A, HLA-B, and HLA-C and a modification at T228 in HLA-A, HLA-B, and HLA-C.

In some embodiments of the method of making a hypoimmunogenic cell of the disclosure, the modification at D227 is D227K and the modification at T228 is T228A.

In some embodiments of the method of making a hypoimmunogenic cell of the disclosure, the one or more genetic modifications comprises one or more in-frame stop codon mutations.

In some embodiments of the method of making a hypoimmunogenic cell of the disclosure, the one or more in-frame stop codon mutation is introduced using a small guide RNA comprising a sequence selected from the group consisting of:

(SEQ ID NO: 41)
CCAGAAGUGGGCGGCUGUGG,
(SEQ ID NO: 42)
AGCAGGAGGGGCCGGAGUAU,
(SEQ ID NO: 43)
GCAGGACGCCUACGACGGCA,
(SEQ ID NO: 50)
UACCGGCAGGACGCCUACGA,
(SEQ ID NO: 51)
GGAGCAGCGGAGAGUCUACC,
(SEQ ID NO: 52)
CGCUGCAGCGCACGGGUACC,
(SEQ ID NO: 53)
GACCUGGCAGCGGGAUGGGG,
(SEQ ID NO: 58)
CGAGCCAGAAGAUGGAGCCG,
and
(SEQ ID NO: 59)
UUACCCCAUCUCAGGGUGAG.

In some embodiments of the method of making a hypoimmunogenic cell of the disclosure, the one or more genetic modification disrupts HLA transcript splicing and/or translation.

In some embodiments of the method of making a hypoimmunogenic cell of the disclosure, the genetic modification is introduced using a base editor and one or more small guide RNAs comprising the sequence of CCUUACCCCAUCUCAGGGUG (SEQ ID NO: 57), UGACGGCCAUCCUCGGCGUC (SEQ ID NO: 60), or CUACGUAGGGUCCUUCAUCC (SEQ ID NO: 61).

In some embodiments of the method of making a hypoimmunogenic cell of the disclosure, the method further comprises introducing into the cell one or more polynucleotides encoding one or more exogenous HLA genes, optionally wherein the HLA genes comprise one or more mutations.

In some embodiments of the method of making a hypoimmunogenic cell of the disclosure, the one or more exogenous HLA genes comprise HLA-E gene.

In some embodiments of the method of making a hypoimmunogenic cell of the disclosure, the genetic modification is introduced using a gene editing technique, optionally wherein the gene editing technique is selected from the group consisting of CRISPR-Cas9 gene editing, prime editing, and base editing.

In some embodiments of the method of making a hypoimmunogenic cell of the disclosure, the cell is differentiated or derived from a stem cell, optionally wherein the stem cell is an induced pluripotent stem cell (iPSC).

In some embodiments of the method of making a hypoimmunogenic cell of the disclosure, the cell is an immune cell.

In some embodiments of the method of making a hypoimmunogenic cell of the disclosure, the hypoimmunogenic cell is selected from the group consisting of: a T cell, a B cell, an NK cell, a dendritic cell, and a macrophage.

In some embodiments of the method of making a hypoimmunogenic cell of the disclosure, the method further comprises introducing into the cell a polynucleotide encoding a cell surface receptor, optionally wherein the cell surface receptor is a chimeric receptor.

The disclosure provides a pharmaceutical composition comprising (i) the hypoimmunogenic cell of the disclosure or (ii) the hypoimmunogenic cell made according to the method of the disclosure, and a pharmaceutically acceptable excipient.

The disclosure provides a method of treating a disease or condition in a subject in need thereof, comprising administering to the subject the pharmaceutical composition of the disclosure.

In some embodiments of the method of treating a disease or condition of the disclosure, the disease or condition is selected from the group consisting of cancer, an infectious disease, myocardial infarction, blindness, spinal cord injury, ALS, Parkinson's disease, Huntington's disease, Alzheimer's disease, enteric neuropathy, multiple sclerosis, osteoarthritis, skin disease, diabetes, liver disease, osteoporosis, DiGeorge syndrome, kidney disease and an immune disorder.

In some embodiments of the method of treating a disease or condition of the disclosure, the disease or condition is cancer or an autoimmune condition.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 provides a schematic showing T cell (Barrier 1) and NK cell (Barrier 2) cytotoxicity against an allogeneic cell product. Targeted mutations in MHC-I genes advantageously prevent CD8+ T cell interaction while reducing NK cell activation in response to missing-self recognition.

FIG. 2 provides a schematic listing some of the advantages of the use of DNA prime editing and base editing techniques over the use of CRISPR/Cas9 editing.

FIG. 3 provides a schematic showing a targeted introduction of a A245V mutation in the conserved alpha3 domain of MHC-1 genes (HLA-A, HLA-B, and HLA-C), which prevents induction of an NK cell missing-self response without the need to express a separate NK cell suppressor.

FIG. 4 provides a schematic showing the introduction of in frame stop mutations in the conserved alpha3 domain of MHC-1 genes (HLA-A, HLA-B, and HLA-C) in combination with HLA-E overexpression.

FIG. 5 provides histograms showing HLA-A cell surface expression in NK cells edited with a cytosine base editor (BE4) or a adenine base editor (ABE8e) and sgRNA, as quantified by flow cytometry.

FIG. 6 provides bar graphs showing the percentage of cells in which the MHC-I genes HLA-A, HLA-B, HLA-C, HLA-H, HLA-K, and HLA-L were successfully edited using a cytosine base editor (BE4) or a adenine base editor (ABE8e).

FIG. 7 provides flow cytometry pseudo-color plots showing GFP and HLA-A/B/C expression in GFP+ NALM6 cells in the following conditions: unstained control, WT NALM6 cells, and NALM6 cells in which HLA-1 is knocked out.

FIG. 8 provides flow cytometry histograms showing the expression levels of HLA-A/B/C or HLA-E in HLA-1 knockout cells overexpressing various HLA molecules or variants thereof before and after magnetic bead selection. FIG. 8A shows cell surface HLA-A/B/C expression in HLA-1 knockout cells overexpressing WT HLA-A (ΔHLA-1+HLA-A WT OE), mutant HLA-A-A245V (ΔHLA-1+HLA-A A245V OE), or double mutant HLA-A-D227K/T228A (ΔHLA-1+HLA-A D227K/T228A OE). FIG. 8B shows cell surface HLA-E expression in HLA-1 knockout cells overexpressing HLA-E (ΔHLA-1+HLA-E OE).

FIG. 9 provides bar graphs of the target count of NALM6 target cells, including WT NALM6 cells, NALM6 ΔHLA-1 (NALM6-ΔMHCI) cells and NALM6 ΔHLA-1 cells overexpressing WT HLA-A (+HLA-A), mutant HLA-A-A245V (+A→V), double mutant HLA-A-D227K/T228A (+DT→KA), and HLA-E (+HLA-E) cocultured with NK cells. FIG. 9A provides target counts after coculturing with conventional NK (cNK) cells. FIG. 9B provides target counts after coculturing with memory NK (NK-101) cells.

FIG. 10 provides flow cytometry pseudo-color plots showing CellTrace Violet (CTV) fluorescent stains in T cells after coculturing with WT NALM6 cells, NALM6 cells with beta-2-microglobulin knocked out (B2M KO) and NALM6 cells with MHC Class I genes knocked out (HLA-1 KO).

FIG. 11 provides a bar graph with summary data and a chart with individual donor data (n=11) showing the percent of T cell proliferation, normalized to ΔHLA-1 only, for T cells after coculturing with WT NALM6 cells or NALM6 ΔHLA-1 cells overexpressing WT HLA-A, mutant HLA-A-A245V, double mutant HLA-A-D227K/T228A, or WT HLA-E.

FIG. 12 provides graphs showing T cell cytotoxicity against WT NALM6 target cells or NALM6 ΔHLA-1 cells or NALM6 ΔHLA-1 cells overexpressing WT HLA-A, mutant HLA-A-A245V, double mutant HLA-A-D227K/T228A, or WT HLA-E. FIG. 12A provides target cell counts per image as measured by Incucyte over 72 hours. FIG. 12B provides a bar graph showing % target survival at 48 hours.

FIG. 13 provides a table showing the advantages of the hypoimmune cells of the present disclosure over other approaches to mitigate rejection of allogeneic cells.

### DETAILED DESCRIPTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### Overview

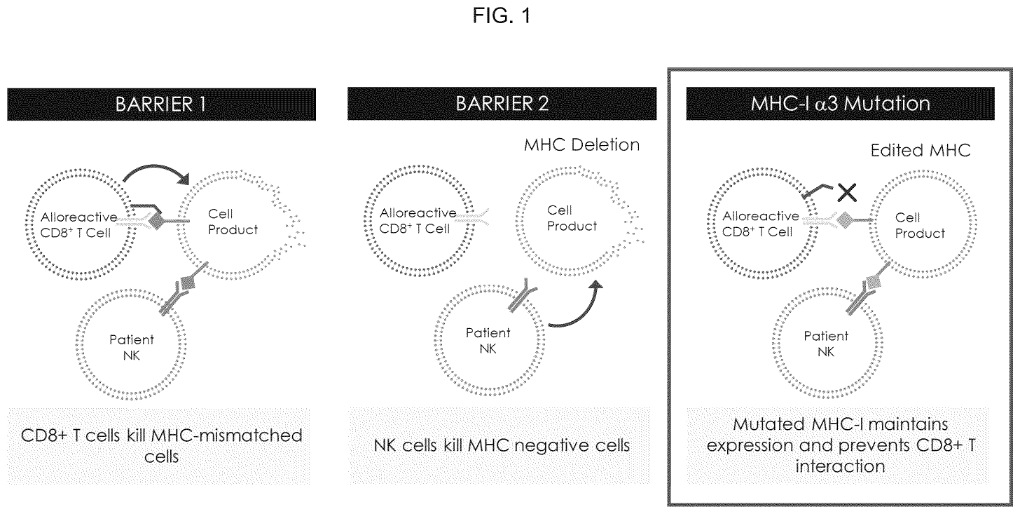

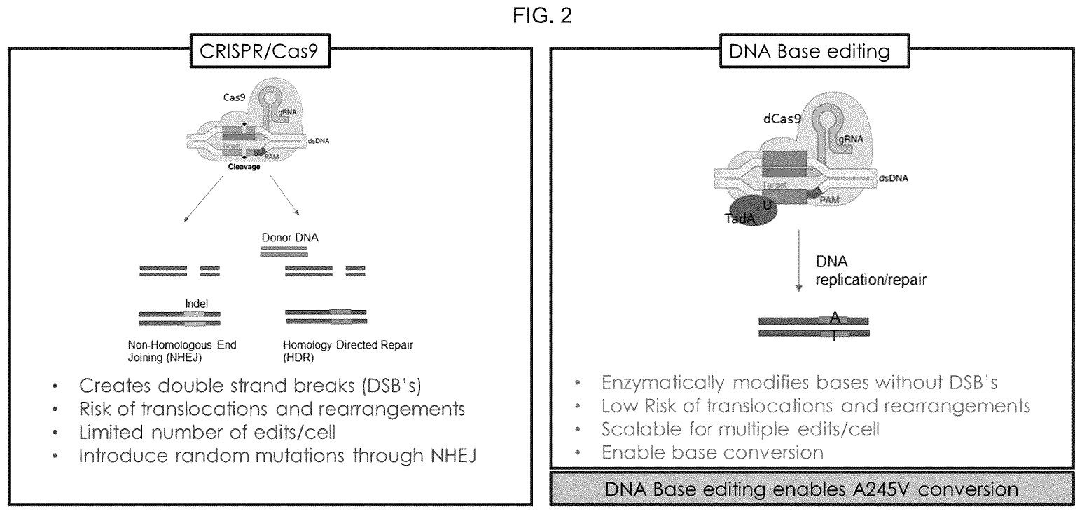

The present disclosure relates to improved allogeneic cellular therapies that effectively abrogate alloreactivity without triggering an NK cell missing self response. Allogeneic cell products are commonly rejected by recipient CD8+ T cells due to a mismatch in donor and recipient MHC (Barrier 1 of FIG. 1). While deletion of MHC prevents the transplanted cells from being targeted by the recipient's T cells, recipient NK cells are activated by the MHC−/− allogeneic cells and kill the MHC-negative cells (Barrier 2 of FIG. 1). The present disclosure provides new compositions and methods to overcome these barriers by providing cells in which MHC-I is mutated. In some embodiments, expression of the mutated MHC-I molecules (MHC-I α3 Mutation) reduces T cell-mediated allo-reactivity by disrupting binding between the CD8 coreceptor and MHC, and expression of MHC prevents triggering of a NK cell missing-self response. The allogeneic cells described herein take advantage from targeted gene editing techniques such as prime editing and/or base editing to specifically disrupt the binding interactions between the MHC-I and CD8 T cell co-receptors (FIG. 2). In some embodiments of the present disclosure, one or more genetic modifications is introduced in one or more alleles of the HLA-A, HLA-B, HLA-C, HLA-E, HLA-F and/or HLA-G genes in order to disrupt binding between the TCR/CD8 coreceptor on recipient T cells and the allo-MHC complex on the transferred cells while preserving binding between NK cell receptors and MHC-I, thereby preventing alloreactive rejection of transferred cells. In some embodiments, the one or more genetic modifications is in HLA-A. In some embodiments, the one or more genetic modifications is in HLA-B. In some embodiments, the one or more genetic modifications is in HLA-C. In some embodiments, the one or more genetic modifications is in HLA-A and HLA-B. In some embodiments, the one or more genetic modifications is in HLA-A and HLA-C. In some embodiments, the one or more genetic modifications is in HLA-A, HLA-B, and HLA-C. In some embodiments, the one or more genetic modifications is in HLA-A, HLA-B, HLA-C, HLA-E, HLA-F, and HLA-G. In some embodiments, the one or more genetic modifications is in HLA-A, HLA-B, HLA-C, HLA-E, and HLA-F. In some embodiments, the one or more genetic modifications is in HLA-B, HLA-C, HLA-E, HLA-F, and HLA-G. In some embodiments, the one or more genetic modifications is in HLA-A, HLA-C, HLA-E, HLA-F, and HLA-G. In some embodiments, the one or more genetic modifications is in HLA-A, HLA-B, HLA-E, HLA-F, and HLA-G. In some embodiments, the one or more genetic modifications is in HLA-A, HLA-B, HLA-C, HLA-F, and HLA-G. In some embodiments, the one or more genetic modifications is in HLA-A, HLA-B, HLA-C, HLA-E, and HLA-G.

In some embodiments, HLA-E cell surface expression on the allogeneic donor cells is preserved because MHC-I deletion is not mediated by beta-2-microglobulin (B2M) gene knockout. In some embodiments, HLA-E cell surface expression on the allogeneic donor cells is preserved because the one or more genetic modifications does not target the beta-2-microglobulin (B2M) gene. In some embodiments, expression of HLA-E inhibits NK cell mediated killing of allogeneic cells.

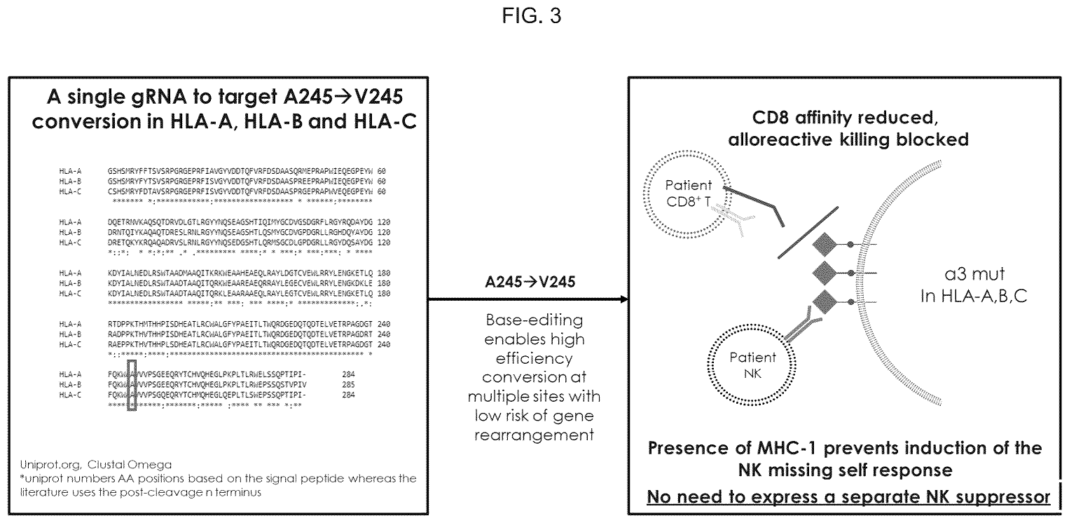

In some embodiments, the one or more genetic modifications comprises a single amino acid substitution. In some embodiments, the one or more genetic modifications is introduced within conserved regions of HLA-A, HLA-B and HLA-C. In some embodiments, the one or more genetic modifications is introduced in the alpha3 domain. In some embodiments, the one or more genetic modifications in the alpha3 domain targets alanine at amino acid residue 245 in HLA-A, HLA-B and HLA-C. In some embodiments, the one or more genetic modifications in the alpha3 domain is an A245V mutation introduced in HLA-A, HLA-B and HLA-C (FIG. 3). In some embodiments, the one or more genetic modifications in the alpha3 domain targets aspartic acid at amino acid residue 227 in HLA-A, HLA-B and HLA-C. In some embodiments, the one or more genetic modifications in the alpha3 domain is an D227K mutation introduced in HLA-A, HLA-B and HLA-C. In some embodiments, the one or more genetic modifications in the alpha3 domain targets threonine at amino acid residue 228 in HLA-A, HLA-B and HLA-C. In some embodiments, the one or more genetic modifications in the alpha3 domain is an T228A mutation introduced in HLA-A, HLA-B and HLA-C. In some embodiments, the one or more genetic modifications in the alpha3 domain is an D227K and an T228A mutation introduced in HLA-A, HLA-B and HLA-C. In some embodiments, the one or more genetic modifications is introduced in the alpha1 domain. In some embodiments, the one or more genetic modifications in the alpha1 domain targets lysine at amino acid residue 66 in HLA-A, HLA-B and HLA-C. In some embodiments, the one or more genetic modifications in the alpha1 domain is an K66A mutation introduced in HLA-A, HLA-B and HLA-C. In some embodiments, the one or more genetic modifications in the alpha1 domain targets arginine at amino acid residue 65 in HLA-A, HLA-B and HLA-C. In some embodiments, the one or more genetic modifications in the alpha1 domain is an R65A mutation introduced in HLA-A, HLA-B and HLA-C. In some embodiments, a combination of these mutations is introduced. These mutations advantageously reduce or prevent alloreactive rejection of engineered cells for use in cellular therapy. In some embodiments, the same or similar mutations are introduced into HLA-E, HLA-F and/or HLA-G.

In some embodiments of the allogeneic cell of the disclosure, one or more HLA genes are modified. The modified HLA genes may comprise at least one of HLA-A, HLA-B, HLA-C, HLA-E, HLA-F, HLA-G. In one embodiment, the modified HLA genes includes HLA-A. In one embodiment, the modified HLA genes includes HLA-B. In one embodiment, the modified HLA genes includes HLA-C. In one embodiment, the modified HLA genes includes HLA-E. In one embodiment, the modified HLA genes includes HLA-F. In one embodiment, the modified HLA genes includes HLA-G. In one embodiment, the modified HLA genes includes HLA-A, HLA-B. In one embodiment, the modified HLA genes includes HLA-A, HLA-C. In one embodiment, the modified HLA genes includes HLA-A, HLA-E. In one embodiment, the modified HLA genes includes HLA-A, HLA-F. In one embodiment, the modified HLA genes includes HLA-A, HLA-G. In one embodiment, the modified HLA genes includes HLA-B, HLA-C. In one embodiment, the modified HLA genes includes HLA-B, HLA-E. In one embodiment, the modified HLA genes includes HLA-B, HLA-F. In one embodiment, the modified HLA genes includes HLA-B, HLA-G. In one embodiment, the modified HLA genes includes HLA-C, HLA-E. In one embodiment, the modified HLA genes includes HLA-C, HLA-F. In one embodiment, the modified HLA genes includes HLA-C, HLA-G. In one embodiment, the modified HLA genes includes HLA-E, HLA-F. In one embodiment, the modified HLA genes includes HLA-E, HLA-G. In one embodiment, the modified HLA genes includes HLA-F, HLA-G. In one embodiment, the modified HLA genes includes HLA-A, HLA-B, HLA-C. In one embodiment, the modified HLA genes includes HLA-A, HLA-B, HLA-E. In one embodiment, the modified HLA genes includes HLA-A, HLA-B, HLA-F. In one embodiment, the modified HLA genes includes HLA-A, HLA-B, HLA-G. In one embodiment, the modified HLA genes includes HLA-A, HLA-C, HLA-E. In one embodiment, the modified HLA genes includes HLA-A, HLA-C, HLA-F. In one embodiment, the modified HLA genes includes HLA-A, HLA-C, HLA-G. In one embodiment, the modified HLA genes includes HLA-A, HLA-E, HLA-F. In one embodiment, the modified HLA genes includes HLA-A, HLA-E, HLA-G. In one embodiment, the modified HLA genes includes HLA-A, HLA-F, HLA-G. In one embodiment, the modified HLA genes includes HLA-B, HLA-C, HLA-E. In one embodiment, the modified HLA genes includes HLA-B, HLA-C, HLA-F. In one embodiment, the modified HLA genes includes HLA-B, HLA-C, HLA-G. In one embodiment, the modified HLA genes includes HLA-B, HLA-E, HLA-F. In one embodiment, the modified HLA genes includes HLA-B, HLA-E, HLA-G. In one embodiment, the modified HLA genes includes HLA-B, HLA-F, HLA-G. In one embodiment, the modified HLA genes includes HLA-C, HLA-E, HLA-F. In one embodiment, the modified HLA genes includes HLA-C, HLA-E, HLA-G. In one embodiment, the modified HLA genes includes HLA-C, HLA-F, HLA-G. In one embodiment, the modified HLA genes includes HLA-E, HLA-F, HLA-G. In one embodiment, the modified HLA genes includes HLA-A, HLA-B, HLA-C, HLA-E. In one embodiment, the modified HLA genes includes HLA-A, HLA-B, HLA-C, HLA-F. In one embodiment, the modified HLA genes includes HLA-A, HLA-B, HLA-C, HLA-G. In one embodiment, the modified HLA genes includes HLA-A, HLA-B, HLA-E, HLA-F. In one embodiment, the modified HLA genes includes HLA-A, HLA-B, HLA-E, HLA-G. In one embodiment, the modified HLA genes includes HLA-A, HLA-B, HLA-F, HLA-G. In one embodiment, the modified HLA genes includes HLA-A, HLA-C, HLA-E, HLA-F. In one embodiment, the modified HLA genes includes HLA-A, HLA-C, HLA-E, HLA-G. In one embodiment, the modified HLA genes includes HLA-A, HLA-C, HLA-F, HLA-G. In one embodiment, the modified HLA genes includes HLA-A, HLA-E, HLA-F, HLA-G. In one embodiment, the modified HLA genes includes HLA-B, HLA-C, HLA-E, HLA-F. In one embodiment, the modified HLA genes includes HLA-B, HLA-C, HLA-E, HLA-G. In one embodiment, the modified HLA genes includes HLA-B, HLA-C, HLA-F, HLA-G. In one embodiment, the modified HLA genes includes HLA-B, HLA-E, HLA-F, HLA-G. In one embodiment, the modified HLA genes includes HLA-C, HLA-E, HLA-F, HLA-G. In one embodiment, the modified HLA genes includes HLA-A, HLA-B, HLA-C, HLA-E, HLA-F. In one embodiment, the modified HLA genes includes HLA-A, HLA-B, HLA-C, HLA-E, HLA-G. In one embodiment, the modified HLA genes includes HLA-A, HLA-B, HLA-C, HLA-F, HLA-G. In one embodiment, the modified HLA genes includes HLA-A, HLA-B, HLA-E, HLA-F, HLA-G. In one embodiment, the modified HLA genes includes HLA-A, HLA-C, HLA-E, HLA-F, HLA-G. In one embodiment, the modified HLA genes includes HLA-B, HLA-C, HLA-E, HLA-F, HLA-G. In one embodiment, the modified HLA genes includes HLA-A, HLA-B, HLA-C, HLA-E, HLA-F, HLA-G.

In some embodiments of the allogeneic cell of the disclosure, the allogeneic cell comprises one or more HLA molecules comprising at least one modified amino acid residue selected from A245, D227, T228, K66, R65. In one embodiment, the modified amino acid residues include A245. In one embodiment, the modified amino acid residues include D227. In one embodiment, the modified amino acid residues include T228. In one embodiment, the modified amino acid residues include K66. In one embodiment, the modified amino acid residues include R65. In one embodiment, the modified amino acid residues include A245, D227. In one embodiment, the modified amino acid residues include A245, T228. In one embodiment, the modified amino acid residues include A245, K66. In one embodiment, the modified amino acid residues include A245, R65. In one embodiment, the modified amino acid residues include D227, T228. In one embodiment, the modified amino acid residues include D227, K66. In one embodiment, the modified amino acid residues include D227, R65. In one embodiment, the modified amino acid residues include T228, K66. In one embodiment, the modified amino acid residues include T228, R65. In one embodiment, the modified amino acid residues include K66, R65. In one embodiment, the modified amino acid residues include A245, D227, T228. In one embodiment, the modified amino acid residues include A245, D227, K66. In one embodiment, the modified amino acid residues include A245, D227, R65. In one embodiment, the modified amino acid residues include A245, T228, K66. In one embodiment, the modified amino acid residues include A245, T228, R65. In one embodiment, the modified amino acid residues include A245, K66, R65. In one embodiment, the modified amino acid residues include D227, T228, K66. In one embodiment, the modified amino acid residues include D227, T228, R65. In one embodiment, the modified amino acid residues include D227, K66, R65. In one embodiment, the modified amino acid residues include T228, K66, R65. In one embodiment, the modified amino acid residues include A245, D227, T228, K66. In one embodiment, the modified amino acid residues include A245, D227, T228, R65. In one embodiment, the modified amino acid residues include A245, D227, K66, R65. In one embodiment, the modified amino acid residues include A245, T228, K66, R65. In one embodiment, the modified amino acid residues include D227, T228, K66, R65. In one embodiment, the modified amino acid residues include A245, D227, T228, K66, R65.

In some embodiments of the methods of the disclosure, one or more HLA genes are modified. The modified HLA genes may comprise at least one of HLA-A, HLA-B, HLA-C, HLA-E, HLA-F, HLA-G. In one embodiment, the modified HLA genes includes HLA-A. In one embodiment, the modified HLA genes includes HLA-B. In one embodiment, the modified HLA genes includes HLA-C. In one embodiment, the modified HLA genes includes HLA-E. In one embodiment, the modified HLA genes includes HLA-F. In one embodiment, the modified HLA genes includes HLA-G. In one embodiment, the modified HLA genes includes HLA-A, HLA-B. In one embodiment, the modified HLA genes includes HLA-A, HLA-C. In one embodiment, the modified HLA genes includes HLA-A, HLA-E. In one embodiment, the modified HLA genes includes HLA-A, HLA-F. In one embodiment, the modified HLA genes includes HLA-A, HLA-G. In one embodiment, the modified HLA genes includes HLA-B, HLA-C. In one embodiment, the modified HLA genes includes HLA-B, HLA-E. In one embodiment, the modified HLA genes includes HLA-B, HLA-F. In one embodiment, the modified HLA genes includes HLA-B, HLA-G. In one embodiment, the modified HLA genes includes HLA-C, HLA-E. In one embodiment, the modified HLA genes includes HLA-C, HLA-F. In one embodiment, the modified HLA genes includes HLA-C, HLA-G. In one embodiment, the modified HLA genes includes HLA-E, HLA-F. In one embodiment, the modified HLA genes includes HLA-E, HLA-G. In one embodiment, the modified HLA genes includes HLA-F, HLA-G. In one embodiment, the modified HLA genes includes HLA-A, HLA-B, HLA-C. In one embodiment, the modified HLA genes includes HLA-A, HLA-B, HLA-E. In one embodiment, the modified HLA genes includes HLA-A, HLA-B, HLA-F. In one embodiment, the modified HLA genes includes HLA-A, HLA-B, HLA-G. In one embodiment, the modified HLA genes includes HLA-A, HLA-C, HLA-E. In one embodiment, the modified HLA genes includes HLA-A, HLA-C, HLA-F. In one embodiment, the modified HLA genes includes HLA-A, HLA-C, HLA-G. In one embodiment, the modified HLA genes includes HLA-A, HLA-E, HLA-F. In one embodiment, the modified HLA genes includes HLA-A, HLA-E, HLA-G. In one embodiment, the modified HLA genes includes HLA-A, HLA-F, HLA-G. In one embodiment, the modified HLA genes includes HLA-B, HLA-C, HLA-E. In one embodiment, the modified HLA genes includes HLA-B, HLA-C, HLA-F. In one embodiment, the modified HLA genes includes HLA-B, HLA-C, HLA-G. In one embodiment, the modified HLA genes includes HLA-B, HLA-E, HLA-F. In one embodiment, the modified HLA genes includes HLA-B, HLA-E, HLA-G. In one embodiment, the modified HLA genes includes HLA-B, HLA-F, HLA-G. In one embodiment, the modified HLA genes includes HLA-C, HLA-E, HLA-F. In one embodiment, the modified HLA genes includes HLA-C, HLA-E, HLA-G. In one embodiment, the modified HLA genes includes HLA-C, HLA-F, HLA-G. In one embodiment, the modified HLA genes includes HLA-E, HLA-F, HLA-G. In one embodiment, the modified HLA genes includes HLA-A, HLA-B, HLA-C, HLA-E. In one embodiment, the modified HLA genes includes HLA-A, HLA-B, HLA-C, HLA-F. In one embodiment, the modified HLA genes includes HLA-A, HLA-B, HLA-C, HLA-G. In one embodiment, the modified HLA genes includes HLA-A, HLA-B, HLA-E, HLA-F. In one embodiment, the modified HLA genes includes HLA-A, HLA-B, HLA-E, HLA-G. In one embodiment, the modified HLA genes includes HLA-A, HLA-B, HLA-F, HLA-G. In one embodiment, the modified HLA genes includes HLA-A, HLA-C, HLA-E, HLA-F. In one embodiment, the modified HLA genes includes HLA-A, HLA-C, HLA-E, HLA-G. In one embodiment, the modified HLA genes includes HLA-A, HLA-C, HLA-F, HLA-G. In one embodiment, the modified HLA genes includes HLA-A, HLA-E, HLA-F, HLA-G. In one embodiment, the modified HLA genes includes HLA-B, HLA-C, HLA-E, HLA-F. In one embodiment, the modified HLA genes includes HLA-B, HLA-C, HLA-E, HLA-G. In one embodiment, the modified HLA genes includes HLA-B, HLA-C, HLA-F, HLA-G. In one embodiment, the modified HLA genes includes HLA-B, HLA-E, HLA-F, HLA-G. In one embodiment, the modified HLA genes includes HLA-C, HLA-E, HLA-F, HLA-G. In one embodiment, the modified HLA genes includes HLA-A, HLA-B, HLA-C, HLA-E, HLA-F. In one embodiment, the modified HLA genes includes HLA-A, HLA-B, HLA-C, HLA-E, HLA-G. In one embodiment, the modified HLA genes includes HLA-A, HLA-B, HLA-C, HLA-F, HLA-G. In one embodiment, the modified HLA genes includes HLA-A, HLA-B, HLA-E, HLA-F, HLA-G. In one embodiment, the modified HLA genes includes HLA-A, HLA-C, HLA-E, HLA-F, HLA-G. In one embodiment, the modified HLA genes includes HLA-B, HLA-C, HLA-E, HLA-F, HLA-G. In one embodiment, the modified HLA genes includes HLA-A, HLA-B, HLA-C, HLA-E, HLA-F, HLA-G.

In some embodiments of the methods of the disclosure, the allogeneic cell comprises one or more HLA molecules comprising at least one modified amino acid residue selected from A245, D227, T228, K66, R65. In one embodiment, the modified amino acid residues include A245. In one embodiment, the modified amino acid residues include D227. In one embodiment, the modified amino acid residues include T228. In one embodiment, the modified amino acid residues include K66. In one embodiment, the modified amino acid residues include R65. In one embodiment, the modified amino acid residues include A245, D227. In one embodiment, the modified amino acid residues include A245, T228. In one embodiment, the modified amino acid residues include A245, K66. In one embodiment, the modified amino acid residues include A245, R65. In one embodiment, the modified amino acid residues include D227, T228. In one embodiment, the modified amino acid residues include D227, K66. In one embodiment, the modified amino acid residues include D227, R65. In one embodiment, the modified amino acid residues include T228, K66. In one embodiment, the modified amino acid residues include T228, R65. In one embodiment, the modified amino acid residues include K66, R65. In one embodiment, the modified amino acid residues include A245, D227, T228. In one embodiment, the modified amino acid residues include A245, D227, K66. In one embodiment, the modified amino acid residues include A245, D227, R65. In one embodiment, the modified amino acid residues include A245, T228, K66. In one embodiment, the modified amino acid residues include A245, T228, R65. In one embodiment, the modified amino acid residues include A245, K66, R65. In one embodiment, the modified amino acid residues include D227, T228, K66. In one embodiment, the modified amino acid residues include D227, T228, R65. In one embodiment, the modified amino acid residues include D227, K66, R65. In one embodiment, the modified amino acid residues include T228, K66, R65. In one embodiment, the modified amino acid residues include A245, D227, T228, K66. In one embodiment, the modified amino acid residues include A245, D227, T228, R65. In one embodiment, the modified amino acid residues include A245, D227, K66, R65. In one embodiment, the modified amino acid residues include A245, T228, K66, R65. In one embodiment, the modified amino acid residues include D227, T228, K66, R65. In one embodiment, the modified amino acid residues include A245, D227, T228, K66, R65.

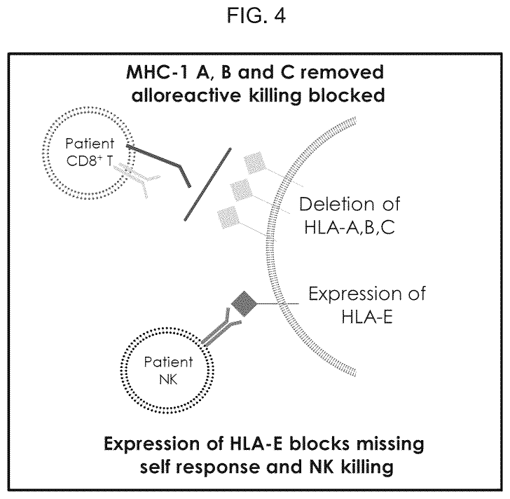

In some embodiments, the one or more genetic modifications comprises an in frame stop codon mutation. In some embodiments, the in frame stop codon mutation is introduced within conserved regions of HLA-A, HLA-B and HLA-C. In some embodiments, the in frame stop codon mutation is introduced within the alpha1 domain of HLA-A, HLA-B and HLA-C. In some embodiments, the in frame stop codon mutation is introduced within the alpha2 domain of HLA-A, HLA-B and HLA-C. In some embodiments, the in frame stop codon mutation is introduced within the alpha3 domain of HLA-A, HLA-B and HLA-C. This strategy of MHC deletion as described herein does not affect the B2M gene. Therefore, the HLA-E gene can be re-expressed with a single chain transgene rather than a triple-chain construct encoding HLA-E, B2M, and peptide. Therefore, deletion of MHC-I expression in this way allows for a simple and improved path to re-expression of HLA-E, or of mutant HLA-A, HLA-B, and/or HLA/C transgenes (e.g., HLA genes carrying one or more genetic modifications such as A245V, D227K, T228A, and/or other mutations described herein or a combination thereof). FIG. 4 provides an exemplary schematic of blocking alloreactive T cell killing by deletion of HLA-A, HLA-B, and HLA-C and blocking alloreactive NK cell killing with transgene expression of HLA-E.

In some embodiments, the one or more genetic modifications comprise modifications that result in in-frame stop codon mutations, introduced using one or more guide RNAs specific for a cytosine base editor (e.g. BE3, BE4) comprising a sequence selected from the group consisting of:

(SEQ ID NO. 50)
UACCGGCAGGACGCCUACGA,
(SEQ ID NO. 51)
GGAGCAGCGGAGAGUCUACC,
(SEQ ID NO. 52)
CGCUGCAGCGCACGGGUACC,
(SEQ ID NO. 53)
GACCUGGCAGCGGGAUGGGG,
(SEQ ID NO. 54)
GGAGGACCAGACCCAGGACA,
(SEQ ID NO. 55)
GACCCAGGACACGGAGCUCG,
and
(SEQ ID NO. 56)
CACACCAUCCAGAUAAUGUA.

In some embodiments, the genetic modifications are introduced with mRNA encoding a cytosine base editor (such as BE4) and using one or more sgRNA guides comprising a sequence selected from the group consisting of:

(SEQ ID NO. 50)
UACCGGCAGGACGCCUACGA,
(SEQ ID NO. 51)
GGAGCAGCGGAGAGUCUACC,
(SEQ ID NO. 52)
CGCUGCAGCGCACGGGUACC,
(SEQ ID NO. 53)
GACCUGGCAGCGGGAUGGGG,
and
(SEQ ID NO. 57)
CCUUACCCCAUCUCAGGGUG [Splice donor exon 6 (SDe6)].

In some embodiments, the genetic modifications are introduced with mRNA encoding a cytosine base editor (such as BE4) and using one or more sgRNA guides comprising a sequence selected from the group consisting of:

(SEQ ID NO. 58)
CGAGCCAGAAGAUGGAGCCG [Stop1],
(SEQ ID NO. 59)
UUACCCCAUCUCAGGGUGAG [Stop2],
and
(SEQ ID NO. 60)
UGACGGCCAUCCUCGGCGUC [Start].

In some embodiments, the genetic modifications are introduced with mRNA encoding a adenine base editor (such as ABE8e) and using one or more sgRNA guides comprising a

(SEQ ID NO. 61)
CUACGUAGGGUCCUUCAUCC [Splice acceptor exon 3 (SAe3)],
(SEQ ID NO. 57)
CCUUACCCCAUCUCAGGGUG [SDe6],
and
(SEQ ID NO. 60)
UGACGGCCAUCCUCGGCGUC [Start].

### General Methods & Definitions

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

Before the present methods and compositions are described, it is to be understood that this invention is not limited to a particular method or composition described. The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

As used herein, “allogeneic” refers to the genetic dissimilarity of a host organism and a cellular transplant where an immune response is generated. Typically, the genetic dissimilarity results in a mismatch between HLA class I and class II molecules between the host and the donor cellular transplant.

By “hypoimmunogenic,” it is meant that the cell elicits an immune response from an allogeneic host or allogeneic cell that is less than what would be elicited by a parental cell or an equivalent naturally occurring cell that does not comprise the one or more modifications. In some instances, the cell elicits a reduced immune response in vivo, i.e., in a subject. In some instances, the cell elicits a reduced immune response in vitro or ex vivo, e.g., in a cell culture setting. In some embodiments, the immune response is a response mounted by leukocytes (e.g., T cells). In some embodiments, the immune response is a response mounted by allogeneic leukocytes (e.g., T cells). In some embodiments, the immune response is a response mounted by NK cells. In some embodiments, the immune response is a response mounted by allogeneic NK cells. In some embodiments, the immune response is reduced to some degree, such as, e.g., 2-fold or more, 3-fold or more, or 4-fold or more, in some cases 5-fold, 6-fold, 7-fold, 8-fold, 9-fold, or 10-fold or more, in certain cases, to negligible levels or levels that are undetectable by methods known in the art for measuring such responses. In some embodiments, the immune response is reduced by at least 20%, at least 30%, at least 40%, at least 50%, at least 60%, at least 70%, at least 80%, at least 90%, up to 100% using methods known in the art for measuring such responses. Such cells can better evade rejection by the host immune system when engrafted into a host.

As used herein, “genetic modification” refers to a site of genomic DNA that has been genetically edited or manipulated using any molecular biological method, e.g., methods described herein, e.g., by delivering to a site of genomic DNA an endonuclease and at least one guide RNA (gRNA). Examples of genetic modifications include insertions, deletions, mutations, duplications, inversions, and translocations, and combinations thereof. In some embodiments, a genetic modification is a deletion. In some embodiments, a genetic modification is an insertion. In other embodiments, a genetic modification is an insertion-deletion mutation (or indel), such that the reading frame of the target gene is shifted leading to an altered gene product or no gene product.

By a “non-naturally occurring” cell, it is meant a cell that has been genetically engineered to comprise one or more modifications relative to a parental cell or an equivalent cell, or that is derived from a cell that has been so genetically engineered.

As used herein, the term “deletion”, which may be used interchangeably with the terms “genetic deletion” or “knock-out” or “KO”, generally refers to a genetic modification wherein a site or region of genomic DNA is removed by any molecular biology method, e.g., methods described herein, e.g., by delivering to a site of genomic DNA an endonuclease and at least one gRNA. Any number of nucleotides can be deleted. In some embodiments, a deletion involves the removal of at least one, at least two, at least three, at least four, at least five, at least ten, at least fifteen, at least twenty, or at least 25 nucleotides. In some embodiments, a deletion involves the removal of 10-50, 25-75, 50-100, 50-200, or more than 100 nucleotides. In some embodiments, a deletion involves the removal of an entire target gene. In some embodiments, a deletion involves the removal of part of a target gene, e.g., all or part of a promoter and/or coding sequence of a target gene. In some embodiments, a deletion involves the removal of a transcriptional regulator, e.g., a promoter region, of a target gene. In some embodiments, a deletion involves the removal of all or part of a coding region such that the product normally expressed by the coding region is no longer expressed, is expressed as a truncated form, or expressed at a reduced level. In some embodiments, a deletion leads to a decrease in expression of a gene relative to an unmodified cell. In some embodiments, a deletion can comprise the introduction of one or more genetic modificationss that disrupts gene expression such as the gene editing introduction of stop codons in gene sequences.

As used herein, the terms “insertion” or “integration”, when used in the context of genomic modification and which may be used interchangeably with the terms “genetic insertion” or “knock-in” or “KI”, generally refers to a genetic modification wherein a polynucleotide is introduced or added into a site or region of genomic DNA by any molecular biological method, e.g., methods described herein, e.g., by delivering to a site of genomic DNA an endonuclease and at least one gRNA. In some embodiments, an insertion may occur within or near a site of genomic DNA that has been the site of a prior genetic modification, e.g., a deletion or insertion-deletion mutation. In some embodiments, an insertion occurs at a site of genomic DNA that partially overlaps, completely overlaps, or is contained within a site of a prior genetic modification, e.g., a deletion or insertion-deletion mutation. In some embodiments, an insertion occurs at a safe harbor locus. An insertion may add a genetic function to a host cell, for example, an increase in levels of an RNA or protein. As will be appreciated by those in the art, this can be accomplished in several ways, including adding one or more additional copies of the gene to the host cell or altering a regulatory component of the endogenous gene to increase expression of the protein that is made.

As used herein, the term “vector” refers to a composition capable of transporting a nucleic acid, i.e., DNA or RNA, or protein into a cell. One type of vector is a “plasmid”, which refers to a circular double-stranded DNA loop into which additional nucleic acid segments can be ligated. Another type of vector is a bacterial artificial chromosome, or “BAC”. Another type of vector is a viral vector, wherein nucleic acid segments can be ligated into the viral genome. Another type of vector is a non-viral vector, e.g., a lipid nanoparticle or an exosome. Another type of vector is a synthetic RNA. Certain vectors are capable of autonomous replication in a host cell into which they are introduced (e.g., bacterial vectors having a bacterial origin of replication and episomal mammalian vectors). Other vectors (e.g., non-episomal mammalian vectors, e.g., lentivirus) are integrated into the genome of a host cell upon introduction into the host cell, and thereby are replicated along with the host genome. Other vectors, while not capable of autonomous replication, are capable of being maintained extrachromosomally in a host cell in which they are introduced (e.g., minicircles, the genome of AAV vectors). Other vectors (e.g., non-episomal mammalian vectors, the genome of lentivirus vectors) are integrated into the genome of a host cell upon introduction into the host cell, and thereby are replicated along with the host genome. Vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Other vectors can include transposons delivered in conjunction with a transposase.

As used herein, the term “heterologous” refers to a composition that is non-native to the rest of the entity to which it is being compared. For example, a polynucleotide introduced by genetic engineering techniques into a plasmid or vector derived from a different species, e.g., a viral genome, is a heterologous polynucleotide. As another example, a promoter operatively linked to a coding sequence with which it is not naturally found linked is a heterologous promoter. As a third example, a gene product, e.g., RNA, protein, not normally encoded by a cell in which it is being expressed is a heterologous gene product. As a fourth example, an expression cassette that is not naturally found in a cell is a heterologous expression cassette.

As used herein, the term “expression cassette” refers to a combination of control elements, e.g., promoter, enhancer(s), Kozak consensus sequence, etc. and a gene or genes to which they are operably linked for expression. An “expression vector” refers to a vector, e.g., plasmid, minicircle DNA, bacterial chromosome (BAC), RNA, virus, and the like, that delivers an expression cassette into a cell.

As used herein, the term “expression” refers to the transcription and/or translation of a coding sequence, e.g., an endogenous gene, a heterologous gene, in a cell.

As used herein, the terms “gene” or “coding sequence” refer to a polynucleotide sequence that encodes a gene product and encompasses both naturally occurring polynucleotide sequences and cDNA. A gene may or may not include regions preceding and following the coding region, e.g., 5′ untranslated (5′ UTR) or “leader” sequences and 3′ UTR or “trailer” sequences, or intervening sequences (introns) between individual coding segments (exons).

As used herein, the term “gene product” refers to the expression product of a polynucleotide sequence such as a polypeptide, peptide, protein or RNA including, for example, a messenger RNA (mRNA), a ribozyme, short interfering RNA (siRNA), microRNA (miRNA), small hairpin RNA (shRNA), guide RNA (gRNA), or circular RNA (circRNA). The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component.

As used herein, the terms “operatively linked”, “operably linked”, or “in operable linkage” refers to a juxtaposition of genetic elements on a single polynucleotide, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the term “promoter” refers to a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species-specific. Promoters may be “constitutive,” meaning continually active, or “inducible,” meaning the promoter can be activated or deactivated by the presence or absence of biotic or abiotic factors.

As used herein, the term “enhancer” refers to a cis-acting regulatory element that stimulates, i.e., promotes or enhances, transcription of an adjacent genes. By a “silencer” it is meant a cis-acting regulatory element that inhibits, i.e., reduces or suppresses, transcription of an adjacent gene, e.g., by actively interfering with general transcription factor assembly or by inhibiting other regulatory elements, e.g., enhancers, associated with the gene. Enhancers can function (i.e., can be associated with a coding sequence) in either orientation, over distances of up to several kilobase pairs (kb) from the coding sequence and from a position downstream of a transcribed region. Enhancer sequences influence promoter-dependent gene expression and may be located in the 5′ or 3′ regions of the native gene. Enhancer sequences may or may not be contiguous with the promoter sequence. Likewise, enhancer sequences may or may not be immediately adjacent to the gene sequence. For example, an enhancer sequence may be several thousand base pairs from the promoter and/or gene sequence.

As used herein, the terms “identical” or percent “identity” in the context of two or more nucleotide sequences or amino acid sequences, refers to having the same or having a specified percentage of amino acid residues or nucleotides that are the same, when compared and aligned for maximum correspondence, as measured using one of the sequence comparison algorithms described herein, e.g., the Smith-Waterman algorithm, or by visual inspection.

As used herein, the term “sequence identity” refers to the degree of identity between nucleotides or amino acids in two or more aligned sequences, when aligned using a sequence alignment program. The term “% homology” is used interchangeably herein with the term “% identity” herein and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Sequence identity may be determined by aligning sequences using any of a number of publicly available alignment algorithm tools, e.g., the local homology algorithm of Smith & Waterman, Adv. Appl. Math. 2: 482 (1981), the global homology alignment algorithm of Needleman & Wunsch, J Mol. Biol. 48: 443 (1970), the search for similarity method of Pearson & Lipman, Proc. Nat'l. Acad. Sci. USA 85: 2444 (1988), computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J Mol. Biol. 215: 403-410 (1990), with software that is publicly available through the National Center for Biotechnology Information (www.ncbi.nlm.nih.gov/), or by visual inspection (see generally, Ausubel et al., infra). In some embodiments, any time the term sequence identity is used herein, it refers to such identity as determined using global alignment.

As used herein, the terms “complement” and “complementary” refer to two antiparallel nucleotide sequences capable of pairing with one another upon formation of hydrogen bonds between the complementary base residues in the antiparallel nucleotide sequences.

As used herein, the terms “native”, or “wild type” when used in the context of a polynucleotide or polypeptide herein, refers to a polynucleotide or polypeptide sequence that is found in nature; i.e., that is present in the genome of a wild type virus or cell.

As used herein, the terms “variant”, when used in the context of a polynucleotide or polypeptide herein, refers to a mutant of a native polynucleotide or polypeptide having less than 100% sequence identity with the native sequence or any other native sequence. Such variants may comprise one or more substitutions, deletions, or insertions in the corresponding native gene or gene product sequence. The term “variant” also includes fragments of the native gene or gene product, and mutants thereof, e.g., fragments comprising one or more substitutions, deletions, or insertions in the corresponding native gene or gene product fragment. In some embodiments, the variant retains a functional activity of the native gene product, e.g., ligand binding, receptor binding, protein signaling, etc., as known in the art.

As used herein, the term “fragment,” when referring to a recombinant protein or polypeptide of the invention, refers to a polypeptide having an amino acid sequence which is the same as part of, but not all of, the amino acid sequence of the corresponding full-length protein or polypeptide, which retains at least one of the functions or activities of the corresponding full-length protein or polypeptide. The fragment preferably includes at least 20-100 contiguous amino acid residues of the full-length protein or polypeptide.

As used herein, the terms “biological activity” and “biologically active” refer to the activity attributed to a particular gene product, e.g., RNA or protein, in a cell line in culture or in vivo. For example, the “biological activity” of an RNAi molecule refers to the ability of the molecule to inhibit the production of a polypeptide from a target polynucleotide sequence.

As used herein, the terms “native” or “wild type” when used in the context of a cell herein, refer to a cell that comprises a genome found in nature, i.e., a genome that has not been engineered to comprise a modification. Thus, for example, a somatic cell that has been harvested from an individual would be a wild type cell. A pluripotent stem cell that has been reprogrammed from that somatic cell would also be a wild type cell. In contrast, a somatic cell or a pluripotent stem cell that has been genetically modified would be a “non-naturally occurring” cell.

As used herein, the term “introducing” refers to contacting a cell, tissue, or subject with a vector for the purposes of delivering a DNA, RNA, or protein to the cell or cells. Such administering or introducing may take place in vivo, in vitro or ex vivo. A vector for expression of a gene product may be introduced into a cell by transfection, which typically means insertion of heterologous DNA, RNA or protein into a cell by physical means (e.g., calcium phosphate transfection, electroporation, microinjection or lipofection), or transduction, which typically refers to introduction by way of a virus or a bacteriophage.

As used herein, the terms “transformation,” “transfection,” “transduction,” or “infection” refer to the delivery of a heterologous DNA, RNA or protein to the interior of a cell, e.g., a mammalian cell, an insect cell, a bacterial cell, etc. by a vector. A vector used to “transform,” “transfect,” “transduce,” or “infect” a cell may be a plasmid, minicircle DNA, synthetic RNA, RNP, lipid nanoparticle, extracellular vesicle, exosome, or other vehicles. Typically, a cell is referred to as “transduced”, “infected,” “transfected” or “transformed” dependent on the means used for administration, introduction or insertion of heterologous DNA, RNA, or protein (i.e., the vector) into the cell. The terms “transfected” and “transformed” are used interchangeably herein to refer to the introduction of heterologous DNA, RNA or protein by non-viral methods, e.g., electroporation, calcium chloride transfection, lipofection, etc. The terms “transduced” and “infected” are used interchangeably herein to refer to introduction of the heterologous DNA or RNA to the cell in the context of a viral particle.

The term “host cell”, as used herein refers to a cell which will be or has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original, transduced, infected, transfected or transformed cell and progeny thereof.

As used herein, a “therapeutic” composition refers to a composition that, when administered, confers a beneficial effect on a subject. Thus, for example, a therapeutic cell composition refers to a cell composition that, when grafted into an individual, confers a beneficial effect on the individual in which it is present, or on a mammal in which the cell composition is grafted. Similarly, for example, a therapeutic gene refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that correct a genetic deficiency in a cell or mammal.

The terms “treatment”, “treating” and the like are used herein to generally refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal and includes: (a) preventing the disease from occurring in a subject which may be predisposed to the disease but has not yet been diagnosed as having it; (b) inhibiting the disease, i.e., slowing or arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein to refer to any mammalian subject for whom diagnosis, treatment, or therapy is desired, particularly humans.

### Targeted Genetic Modifications

The present disclosure employs precise genetic modification techniques in order to introduce specific mutations in one or more genes of interest. While the discovery of clustered regularly interspaced short palindromic repeats (CRISPR)-Cas system has revolutionized the field of molecular biology and medicine (Doudna et al., Science. 2014; 346:1258096), it has also several limitations that hinder its use in certain settings. CRISPR-mediated genome editing involves the generation of a Cas9-induced double-strand break that is repaired by non-homologous end joining (N HEJ) mechanisms or by homology directed repair (HDR) (Gallagher et al., ACS Chem. Biol. 2018; 13:397-405; Rouet et al., Mol. Cell. Biol. 1994; 14:8096-8106; Sander et al., Nat. Biotechnol. 2014; 32:347-355). Although HDR can be harnessed to insert a specific DNA template for precise restoration of the DNA sequence, this pathway is characterized by limited efficiency and high rates of undesired insertion or deletion (indel) mutations that nullify the potential benefit from repairing the one or more genetic modifications (Mao et al., Mol. Ther. Nucleic Acids. 2017; 7:53-60). Moreover, due to reliance on homologous recombination, HDR-mediated editing is restricted to dividing cell types, limiting the range of diseases that can be targeted (Bollen et al., Nucleic Acids Res. 2018; 46:6435-6454).

To address some of these limitations, CRISPR/Cas-mediated single-base-pair editing systems have been devised (Nishida et al., Science. 2016; 353:aaf8729; Komor et al., Cell. 2017; 168:20-36). Two classes of DNA base-editors (BEs) have been described thus far: cytosine base-editors (CBEs) and adenine base-editors (ABEs). These BEs can install all four transition mutations. Prime-editors (PEs) represent another addition to the CRISPR genome-engineering toolkit and represents an approach to expand the scope of donor-free precise DNA editing to not only all transition and transversion mutations, but small insertion and deletion mutations as well (Anzalone et al., Nature. 2019; 576:149-157). Collectively, DNA base-editing and prime-editing tools enable precise nucleotide substitutions in a programmable manner, without requiring a donor template.

In some embodiments of the present disclosure, the desired modifications in the HLA-A, HLA-B and HLA-C genes (or other genes of interest) are introduced using a base-editing technique or a prime-editing technique. In some embodiments, the technique is a CRISPR/Cas-mediated single base pair editing system or other similar system. Many such techniques (as well as variations of them) are known and available and can be readily used in accordance with the present disclosure. Some examples are described below for purposes of illustration.

### DNA Base-Editing

Many DNA base-editing and prime-editing techniques are reviewed in Kantor et al. (Int J Mol Sci. 2020 September; 21(17): 6240). As described therein, DNA base-editors encompass two key components: a Cas enzyme for programmable DNA binding and a single-stranded DNA modifying enzyme for targeted nucleotide alteration. Two classes of DNA base-editors have been described: cytosine base-editors and adenine base-editors. Collectively, all four transition mutations (C→T, T→C, A→G, and G→A) can be installed with the many available CRISPR/Cas BEs (e.g., Table 1 of Kantor et al.). Kurt et al. describe the engineering of two novel base-editor architectures that can efficiently induce targeted C-to-G base transversions (Kurt et al. Nat. Biotechnol. 2020 doi: 10.1038/s41587-020-0609-x). In addition, recent studies report dual base-editor systems for combinatorial editing in human cells (Grinewald et al., Nat. Biotechnol. 2020; 38:861-864; Sakata et al., Nat. Biotechnol. 2020; 38:865-869; Zhang et al., Nat. Biotechnol. 2020; 38:856-860). Together, these new base-editors expand the range of DNA base-editors to transversion mutations and may allow for targeting of more complex compound edits than are currently achievable by a single DNA base-editor.

In some embodiments, as shown in FIG. 2, editing of MHC-I is achieved through CRISPR base editing. CRISPR base editing provides advantages over CRISPR/Cas9-mediated gene knockout, such as lower risk of gene translocations and rearrangements when simultaneously targeting multiple genes for editing. In some embodiments, targeted mutation of alanine to valine at position 245 of all the alleles of the MHC-I genes is achieved by base editing.

### A1. Cytosine Base-Editors

The first-generation base-editor (CBE1) was described by Liu and co-workers (Komor et al., Cell. 2017; 168:20-36; Komor et al., Nature. 2016; 533:420-424). It was engineered by fusing a rat-derived cytosine deaminase Apolipoprotein B MRNA Editing Enzyme Catalytic Subunit 1 (APOBEC1) to the amino terminus of catalytically deficient, or “dead”, Cas9 (dCas9). In a narrow window of the non-targeted strand, CBE1 deaminates cytosine to uracil. Uracil is then recognized by cell replication machinery as a thymine, resulting in a C-G to T-A transition. Importantly, although CBE1 mediates efficient, targeted base-editing in vitro (up to 37% editing with a 11% indel formation rate), it is not effective in human cells. This decrease is largely due to cellular-mediated repair of the U-G intermediate in DNA by the base excision repair (BER) pathway. BER of U-G in DNA is initiated by uracil N-glycosylate (UNG), which recognizes the U-G mismatch and cleaves the glycosidic bond between the uracil and the deoxyribose backbone of DNA, resulting in reversion of the U-G intermediate created by the base-editor back to the C-G base pair (Krokan et al., Cold Spring Harbor Perspect. Biol. 2013; 5:a012583; Kunz et al., Cell. Mol. Life Sci. 2009; 66:1021-1038). Keeping in view the low editing efficiency and limitations of CBE1, a series of improved base-editors were developed further. To improve base-editing efficiency, a second-generation cytosine base-editor (CBF2) was developed by fusing an uracil DNA glycosylase inhibitor (UTGI) to the C-terminus of BE., inhibiting the activity of UDG. The inhibition of BER by BE2 resulted in a threefold increase in editing efficiency in human cells (Komor et al., Nature. 2016; 533:420-424). To further improve editing efficacy, BE3 was developed by restoring histidine at position 840 (H1840, HNH catalytic domain) in dCas9 to generate a base-editor that uses Cas9 nickase (nCas9). This variant induces a nick in the G-containing strand of the U-G intermediate (non-edited DNA strand) to bias cellular repair of the intermediate towards a U-A outcome, further converted to T-A during DNA replication. This modification further increased editing efficiency by six-fold in BE3 over BE2. The use of nCas9 also exhibited an increase in indel frequency of 1.1% as compared to 0.1% in BE2; however, this is still a low rate that is much less frequent than indels induced by DSBs.

Further optimization of CBE was performed to reduce indel formation during base-editing, improve editing efficiency, and narrow the editing window. An improved fourth-generation cytosine base-editor (CBE4) was generated by fusing an additional copy of UGI to the N terminus of nCas9 with an optimized 27 bp linker (Kim et al., Nat. Biotechnol. 2017; 35:371). YEE-BE3 was developed by screening several mutations previously reported to modulate the catalytic activity of cytosine deaminases in the APOBEC family to generate an improved rAPOBEC1 with a narrower editing window and reduced “bystander editing” compared to CBE3. Gam, a DNA-binding protein from bacteriophage Mu, can form a complex with free-ends of DBSs, thus preventing NHEJ-mediated repair and reducing indel formation (Bhattacharyya et al., Proc. Natl. Acad. Sci. USA. 2018; 115:E11614-E11622). These changes resulted in BE4-Gam, which is characterized by higher base-editing efficiency, increased product purity, and decreased indel frequency (Komor et al., Elife. 2013; 2:e01222), which may reduce its adaptability towards therapeutic applications.

Koblan et al. added two nuclear localization signals (NLS) to nCas9 and performed codon-optimization and ancestral sequence reconstruction on APOBEC, yielding BE4max and ancBE4max (Koblan et alt, Nat. Biotechnol. 2018; 36:843-846). Another base-editing system, Target-AID (activation-induced cytidine deaminase), was developed and composed of nCas9, Petromyzon marinus cytidine deaminase 1 (pmCDA1), which is similar to rAPOBEC1 in structure and function (Nishida et al., Science. 2016; 353:aaf8729). The use of alternative cytosine deaminase enzymes yields base-editors with alternative sequence motif preference and the ability to efficiently edit methylated cytosines. Liu and colleagues used phase assisted continuous evolution (PACE) to evolve CBEs and generate evoAPOBEC1-BE4max, which can efficiently edit cytosine in G/C sequences (a disfavored context for wild-type APOBEC1 deaminase) and evoFERNY-BE4max, a smaller deaminase that edits efficiently in all tested sequence contests (Thuronyi et al., Nat. Biotechnol. 2019; 37:1070-1079).

To increase the number of targetable bases, researchers have developed base-editors incorporating different CRISPR-associated nuclease enzymes. CBEs based on SpCas9 are limited by their G/C-rich PAM sequence. In order to expand the scope of base-editing, Li et al. generated a Cpf1-based cytosine deaminase base-editor by fusing catalytically inactive LbCpf1 (dLbCpf1) or dAsCpf1 with rAPOBEC1 and UGI (creating dLbCpf1-BEG and dAsCpf1-BEG). A variety of engineered Cas9 variants with altered PAM sequences and improved cleavage specificity have been developed and may allow for further expansion of the targeting scope of CRISPR-base-editing reagents (Chatterjee et al., Nat. Biotechnol. 2020 doi: 10.1038/s41587-020-0517-0; Kim et al., Nat. Biomed. Eng. 2020; 4:111-124; Miller et al., Nat. Biotechnol. 2020; 38:471-481; Kleinstiver et al., Nature. 2015; 523:481-485; Lee et al., Mol. Ther. 2016; 24:645-654; Miller et al., Mo. Ther. 2016; 24:636-644; Zetsche et al., Cell. 2015; 163:759-771; Pausch et al., Science. 2020; 369:333-337). These constructs enable efficient single-vector AAV delivery and may prove especially useful for therapeutic applications that are constrained by viral-vector packaging capacity.

### A2. Adenine Base-Editors

The cytosine base-editor is limited to installing a C-G to T-A mutation, greatly restricting the range of correctable disease-causing mutations. Importantly, methylated cytosines are vulnerable to high rates of spontaneous cytosine deamination (Alsøe et al., Sci. Rep. 2017; 7:1-14) and nearly half of all pathogenic point mutations in principle can be reversed using an ABE to convert an A-T base pair back into a G-C base pair. As such, base-editing capabilities and study of genetic diseases were further expanded by the development of a new class of adenine base-editors that could induce A to G conversions (Gaudelli et al., Nature. 2017; 551:464-471). ABE-mediated DNA editing operates under a similar mechanism as CBE. The ABE-dCas9 fusion binds to a target DNA sequence in a guide RNA-programmed manner, and the deoxyadenosine deaminase domain catalyzes an adenine to inosine transition. In the context of DNA replication, inosine is interpreted as guanine, and the original A-T base pair may be replaced with a G-C base pair at the target site. Unlike cytosine deaminases, ssDNA adenosine deaminase enzymes do not occur in nature. Attempts at utilizing RNA adenosine deaminases to act on DNA resulted in no detectable RNA editing (Gaudelli et al., Nature. 2017; 551:464-471). David Liu and group overcame this limitation through extensive protein engineering and directed evolution of Escherichia coli tRNA adenosine deaminase, TadA (ecTadA). EcTadA converts adenine to inosine in the single-stranded anticodon loop of tRNA^{ARG}, and shares sequence similarity with the APOBEC family. The first-generation adenine base-editors were developed through an antibiotic resistance complementation approach in bacteria. To test TadA on a DNA target, E. coli cells were equipped with TadA mutants and defective antibiotic resistance genes. To grow in the presence of antibiotic, a mutant TadA-dCa9 fusion had to correct the targeted adenine in a mutant chloramphenicol resistance gene. The first-generation ABE (ABE1.2) was generated by fusing the evolved TadA variant (TadA*) to the N-terminus of nCas9 through XTEN (a 16 amino acid linked used in BE3), with the C terminal of nCas9 fused with a nuclear localization signal (TadA*-XTEN-nCas9-NLS). In comparison with cytosine base-editing, adenine base-editing by ABE yields a much cleaner product that has virtually no indels, and there are no reports of significant off-target (A-to-non-G) edits to date. Consistent with this observation, unlike UGI-mediated inhibition of UDG in CBEs, ABE editing in cells lacking alkyl adenine DNA glycosylase (AAG), the enzyme known to recognize and remove inosine in DNA, failed to increase editing efficiency or product purity compared with cells containing wild-type AAG. Indeed, even early generations of ABE recovered ≥99.9% pure product with a negligible rate of indels (≤0.1%) (Gaudelli et al., Nature. 2017; 551:464-471; Yang et al., Protein Cell. 2018; 9:814-819; Huang T. P., et al., Nat. Biotechnol. 2019; 37:626-631).

In its native context, TadA acts as a homodimer, with one monomer catalyzing deamination and the other monomer enabling tRNA substrate binding (Losey et al., Nat. Struct. Mol. Biol. 2006; 13:153-159). To optimize ABEs, Gaudelli et al. engineered a single-chain heterodimer comprised of a wild-type non-catalytic TadA monomer and evolved ecTadA monomer (TadA-TadA*). To improve editing efficiency, further optimization of ABE was performed. Extensive PACE and protein engineering resulted in seventh generation ABEs (ABE7.10), which converted target A-T to G-C efficiently (˜50%) in human cells (Gaudelli et al., Nature. 2017; 551:464-471; Table 1).

Only about one-quarter of pathogenic transition mutations encompass an appropriately located NGG PAM site that facilitates SpCas9-mediated base-editing. Unlike CBEs, which have proven to be broadly customizable with many Cas orthologs, ABEs have shown limited compatibility with Cas9 of any origin other than SpCas9. Although some homologs such as SaCas9 and circularly permuted Cas9 (CP-Cas9) have been adapted (Huang et al., Nat. Biotechnol. 2019; 37:626-631), editing efficiencies are substantially lower than those demonstrated with CBE counterparts (Zuo et al., Science. 2019; 364:289-292). This incompatibility is due to the low DNA-bound residence time of non-SpCas9, coupled with the slow enzymatic rate of deoxyadenosine deaminase. To address this problem, Richter et al. utilized phage-assisted continuous and non-continuous evolution (PACE and PANCE) methods to enhance the catalytic rate of the deoxyadenosine deaminase enzyme by 590-fold compared to that of AB137.10 (Richter et al., Nat. Biotechnol. 2020:1-9. doi: 10.1038/s41587-020-0453-z). This next generation of ABEs, designated ABE8e, shows greatly enhanced activity and compatibility with diverse Cas9 homologs. As expected, the targeting scope of ABE8e also increased off-target RNA and DNA editing. However, the authors show that the off-target editing can be ameliorated by introduction of an additional point-mutation (V106W). Together, ABESe expands the targeting range, editing efficiency and broad functionality of ABEs. It will be interesting to see whether these outcomes will translate in vivo and the eight-generation of ABEs can outperform previously developed base-editor constructs.

### A3. Prime-Editing

Despite the profound capabilities of CBEs and ABEs to edit the DNA, a major limitation of the current base-editing technologies (until recently) has been the ability to generate precise base-edits beyond the four transition mutations. A method to overcome these shortcomings, known as prime-editing, has been described by Anzalone et al. (Anzalone et al., Nature. 2019; 576:149-157). As with CRISPR-mediated base-editing, prime-editing does not rely on DSBs. Prime-editors use an engineered reverse transcriptase fused to Cas9 nickase and a prime-editing guide RNA (pegRNA). Importantly, the pegRNA differs significantly from regular sgRNAs and plays a major role in the system's function. The pegRNA contains not only (a) the sequence complimentary to the target sites that directs nCas9 to its target sequence, but also (b) an additional sequence spelling the desired sequence changes (Anzalone et al., Nature. 2019; 576:149-157). The 5′ of the pegRNA binds to the primer binding site (PBS) region on the DNA, exposing the non-complimentary strand. The unbound DNA of the PAM-containing strand is nicked by Cas9, creating a primer for the reverse transcriptase (RT) that is linked to nCas9. The nicked PAM-strand is then extended by the RT by using the interior of the pegRNA as a template, consequently modifying the target region in a programmable manner. The result of this step is two redundant PAM DNA flaps: the edited 3′ flap that was reverse transcribed from the pegRN A and the original, unedited 5′ flap. The choice of which flap hybridizes with the non-PAM containing DNA-strand is an equilibrium process, in which the perfectly complimentary 5′ would likely be thermodynamically favored. However, the 5′ flaps are preferentially degraded by cellular endonucleases that are ubiquitous during lagging-strand DNA synthesis (Hosfield et al., Cell. 1998; 95:135-146). Finally, the resulting heteroduplex containing the unedited strand and edited 3′ flap is resolved and stably integrated into the host genome via cellular replication and repair process.

The first generation of PEs (PEI) was comprised of Moloney murine leukemia virus reverse transcriptase (M-MLV T), linked to the c-terminus of nCas9 and pegRNA, which was expressed on a second plasmid. The efficiency of PEI reached maximum editing efficiency of 0.7-5.5% (Anzalone et al., Nature. 2019; 576:149-157). To further enhance the efficiency of the reverse transcriptase, Anzalone and colleagues tested different M-MLV RT variants that have been shown to enhance binding, enzyme processivity, and thermostability. As was previously applied to enhance editing in CBE and ABE systems, a separate sgRNA was directed to introduce a nick in the non-edited strand, thus directing DNA repair to that strand using the edited strand as a template. This yielded another generation prime-editor, designated PE3, which performed all 12 possible transition and transversion mutations (24 single-nucleotide substitutions) with average editing efficiencies of 33% (±7.9%) (Anzalone et al., Nature. 2019; 576:149-157). The number of off-target effects observed with PEs was greatly reduced, likely due to the need for complementation at Cas9 binding, PBS binding, and RT product complementation for flap resolution (Anzalone et al., Nature. 2019; 576:149-157). Prime-editing shows other advantages over previous CRISPR-mediated base-editing approaches, including less stringent PAM requirements due to the varied length of the RT template and no “bystander” editing. The prime-editing system represents a significant milestone in the development of a universal method for genome editing, and its clinical adaptation towards the correction of known pathogenic mutations may prove tremendous.

The above base editing and prime editing techniques (as well as related methods and further developments of same) can be readily used by the skilled individual to carry out precise gene editing in accordance with the present disclosure with a reasonable expectation of success.

In certain specific embodiments, for example, the targeted modifications are introduced using a technique described in US 2017/0073670, WO2015/089406, WO2017/070632, WO2018/176009, U.S. Ser. No. 11/268,082, WO2020/191241, WO2020/191248, WO2021/226558, WO2021/072328 and/or WO2021/155065, the contents of which are incorporated herein by reference in their entireties.

### Hypoimmunogenic Cells

The targeted mutations described herein can be carried out in essentially any cell type where it is advantageous to reduce the immunogenicity of the cell. In many embodiments, the hypoimmunogenic cells comprise cells to be used as an allogeneic cell therapy in methods of treating a human disease or disorder, such as cancer.

In some embodiments, the hypoimmunogenic cells of the disclosure will comprise hypoimmunogenic stem cells, such as pluripotent stem cells. The term “pluripotent cells” refers to cells that can self-renew and proliferate while remaining in an undifferentiated state and that can, under the proper conditions, be induced to differentiate into any of the three germ layers: endoderm (e.g., the stomach lining, gastrointestinal tract, lungs, etc.), mesoderm (e.g., muscle, bone, blood, urogenital tissue, etc.) or ectoderm (e.g., epidermal tissues and nervous system tissues). The term “pluripotent cells,” as used herein, encompasses embryonic stem cells and other types of stem cells, including fetal, amniotic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350:13. (2004), incorporated by reference herein in its entirety.) The term “pluripotent stem cells,” as used herein, also encompasses “induced pluripotent stem cells”, “iPSs” or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of reprogramming proteins or by the exogenous application of certain proteins. Methods of generating and characterizing iPS cells are well known in the art and include those described herein and found in Application Nos. US20090047263, US20090068742, US20090191159, US20090227032, US20090246875, and US20090304646, and Zhou et al., Stem Cells 27 (11): 2667-74 (2009); Huangfu et al., Nature Biotechnol. 26 (7): 795 (2008); Woltjen et al., Nature 458 (7239): 766-770 (2009); Zhou et al., Cell Stem Cell 8:381-384 (2009) and Omole and Fakoya, PeerJ. 2018; 6:e4370 (2018), each of which is incorporated by reference in its entirety.

In some embodiments, the hypoimmunogenic cells of the disclosure are hypoimmunogenic somatic cells. A “somatic cell” refers to any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, a somatic cell would include an adult stem cell, a progenitor cell (i.e., a cell that can renew and can differentiate into a specific cell type), a precursor cell (i.e., a cell that can differentiate into one type of cell), or a differentiated cell. As one nonlimiting example, a somatic cell would include both neurons and neural progenitors, the latter of which may be able to naturally give rise to all or some neuronal types of the central nervous system but cannot give rise to all types of cells of the CNS or to the mesoderm or endoderm lineages.

In some embodiments, the hypoimmunogenic somatic cell is selected from the group consisting of a cardiomyocyte, a retinal pigment epithelial cell, a photoreceptor cell, a neural cell, a glial cell, a hepatocyte, a chondrocyte, a keratinocyte, a beta islet cell, a hepatocyte, a parathyroid cell, a thymic epithelial cell, an endothelial cell, a mesenchymal cell, a CD34+ hematopoietic stem cell, a peripheral blood mononuclear cell (PBMC), an immune cell, a cytotoxic T-cell, a helper T-cell, a memory T-cell, a regulatory T-cell, a NK cell, a memory like NK cell, a cytokine induced memory like (CIML) NK cell, a macrophage, tumor infiltrating lymphocyte, a CAR-T cell and a chimeric T cell (TCR-T cells), and the like, including progeny thereof.

The term “somatic cell” as used herein also encompasses somatic cells that are derivatives of a pluripotent stem cell, i.e., the somatic cell is differentiated from a stem cell in vitro (such as an iPSC); and somatic cells that are derivatives of another somatic cell, i.e., the somatic cell is transdifferentiated directly from another somatic cell in vitro. The somatic cell derivatives of hypoimmunogenic cells will likewise be hypoimmunogenic.

In some embodiments of the disclosure, somatic cell derivatives of hypoimmunogenic cells can be essentially any cell type of interest. Thus, for example, the methods of the invention can comprise a step of differentiating hypoimmunogenic pluripotent stem cells into hypoimmunogenic cardiomyocytes, into hypoimmunogenic skeletal muscle, into hypoimmunogenic endothelial cells, into hypoimmunogenic neurons, into hypoimmunogenic retinal cells, e.g. RPE or photoreceptors, into hypoimmunogenic cartilage, into hypoimmunogenic hepatocytes, into hypoimmunogenic beta-like pancreatic cells or islet organoids, into hypoimmunogenic epithelial cells, or into hypoimmunogenic thymic epithelial progenitors, etc.

In other more particular embodiments, the methods of the invention can comprise a step of differentiating hypoimmunogenic pluripotent stem cells (e.g. hypoimmunogenic iPSCs), into hypoimmunogenic immune cells, including hypoimmunogenic T cells (including cytotoxic T-cells, helper T-cells, memory T-cells, regulatory T-cells), NK cells, memory like (CIML) NK cells, macrophages, tumor infiltrating lymphocytes, etc., as well as into hypoimmunogenic CAR-T cells, hypoimmunogenic chimeric T cells (TCR-T cells), and the like, including progeny thereof.

### Natural Killer Cells

In certain preferred embodiments of the present disclosure, the hypoimmunogenic cells are NK cells. Natural killer (NK) cells are an alternative to T cells for allogeneic cellular immunotherapy since they have been administered safely without major toxicity, do not cause graft versus host disease (GvHD), naturally recognize and eliminate malignant cells, and are amendable to cellular engineering.

The term “NK cells” can refer generally to NK cells and subtypes thereof, such as memory NK cells, memory-like (ML) NK cells, and cytokine-induced memory-like (CIML) NK cells, and variations thereof, any of which may be derived from various sources, including peripheral or cord blood cells, stem cells, induced pluripotent stem cells (iPSCs), and immortalized NK cells such as NK-92 cells.

NK cells are traditionally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. Natural killer (NK) cells are an alternative to T cells for allogeneic cellular immunotherapy since they have been administered safely without major toxicity, do not cause graft versus host disease (GvHD), naturally recognize and eliminate malignant cells, and are amendable to cellular engineering.

In addition to their innate cytotoxic and immunostimulatory activity, NK cells constitute a heterogeneous and versatile cell subset, including persistent memory NK populations, in some cases also called memory-like or cytokine-induced-memory-like (CIML) NK cells, that mount robust recall responses. Memory NK cells can be produced by stimulation by pro-inflammatory cytokines or activating receptor pathways, either naturally or artificially (“priming”). Memory NK cells produced by cytokine activation have been used clinically in the setting of leukemia immunotherapy.

Increased CD56, Ki-67, NKG2A, and increased activating receptors NKG2D, NKp30, and NKp44 have been observed in in vivo differentiated memory NK cells. In addition, in vivo differentiation showed modest decreases in the median expression of CD16 and CD11b. Increased frequency of TRAIL, CD69, CD62L, NKG2A, and NKp30-positive NK cells were observed in ML NK cells compared with both ACT and BL NK cells, whereas the frequencies of CD27+ and CD127+NK cells were reduced. Finally, unlike in vitro differentiated ML NK cells, in vivo differentiated ML NK cells did not express CD25.

NK cells may be induced to acquire a memory-like phenotype, for example by priming (preactivation) with combinations of cytokines, such as interleukin-12 (IL-12), IL-15, and IL-18. These cytokine-induced memory-like (CIML) NK cells (CIML-NKs or CIMLs) exhibit enhanced response upon restimulation with the cytokines or triggering via activating receptors. CIML NK cells may be produced by activation with cytokines such as IL-12, IL-15, and IL-18 and/or their related family members, or functional fragments thereof, or fusion proteins comprising functional fragments thereof.

Memory NK cells typically exhibit differential cell surface protein expression patterns when compared to traditional NK cells. Such expression patterns are known in the art and may comprise, for example, increased CD56, CD56 subset CD56dim, CD56 subset CD56bright, CD16, CD94, NKG2A, NKG2D, CD62L, CD25, NKp30, NKp44, and NKp46 (compared to control NK cells) in CIML NK cells (see e.g., Romee et al. Sci Transl Med. 2016 Sep. 21; 8(357):357). Memory NK cells may also be identified by observed in vitro and in vivo properties, such as enhanced effector functions such as cytotoxicity, improved persistence, increased IFN-gamma production, and the like, when compared to a heterogenous NK cell population.

The NK cells used according to the present disclosure can be prepared using any known methodologies. For example, in some embodiments, the isolated NK cells can be activated using cytokines, such as IL-12/15/18. The NK cells can be incubated in the presence of the cytokines for an amount of time sufficient to form cytokine-activated memory-like (ML) NK cells. In some embodiments, methods for preparing ML NK cells to be used according to the present disclosure include those described in WO2020/047299 and WO2020/047473, the contents of which are incorporated herein by reference in their entireties.

### Chimeric Antigen Receptors

In some embodiments, the hypoimmunogenic cells of the present disclosure are immune cells (e.g., T cells, NK cells, etc.) that contain an chimeric antigen receptor (CAR) or other heterologous transgene of interest.

CARs have been widely studied and described. They are generally designed in a modular fashion that comprise an extracellular target-binding domain, a hinge region, a transmembrane domain that anchors the CAR to the cell membrane, and one or more intracellular domains that transmit activation signals. Introduction of CAR molecules into a cell can successfully redirect cells with additional antigen specificity and provides the necessary signals to drive full immune cell activation. Furthermore, the CAR construct moieties can be operably linked with a linker. A linker can be any nucleotide sequence capable of linking the moieties described herein. For example, the linker can be any amino acid sequence suitable for this purpose (e.g., of a length of 9 amino acids).

In some embodiments of the present disclosure, a CAR sequence comprises at least an extracellular domain that binds a target antigen, a transmembrane domain and one or more intracellular signaling domains.

In some embodiments, the hypoimmunogenic cell of the disclosure is a chimeric antigen receptor NK cell as described in WO2020/097164, the contents of which are incorporated herein by reference.

### Illustrative Extracellular Target-Binding Domains

Illustrative extracellular domains can include, for example, receptor proteins, subunits thereof, or targeting antibody fragments against a disease-associated antigen. Such receptor proteins and subunits thereof can include, for example, CD16, CD64, and CD3epsilon. Such targeting antibody fragments can comprise single-chain variable fragments (scFvs). scFvs, as described herein can be any scFv capable of binding to a target antigen or target antigen epitope. For example, the scFvs can target an antigen associated with an infectious disease, a bacterial infection, a virus, or a cancer. scFvs can be against any antigen known in the art, such as those described in U.S. application Ser. No. 15/179,472, and is incorporated by reference in its entirety.

Targeting antibody fragments or scFvs, as described herein, can be against any tumor-associated antigen (TAA). A TAA can be any antigen known in the art to be associated with tumors.

Illustrative examples of scFvs incorporated into CARs can include those that bind, for example, to mesothelin, CD2, CD3, CD4, CD5, CD7, BAFF-R, gpl20, gp41, BCMA, CD123, CD138, CD19, CD20, CD22, CD33, CD38, CD5, IgK, LeY, NKG2D-Ligands, MICA, MICB, ULBP1, ULBP2, ULBP3, ULBP4, ULBP5, ULBP6, ROR1, WT1, c-MET, CAIX, CD133, CD171, CD70, CEA, EGFR, EGFRvIII, EPCAM, EphA2, FAP, GD2, GPC3, Her2, HPV16-E6, IL13Ra2, LeY, MAGEA3, MAGEA4, MART1, MSLN, MUC1, MUC16, NY-ESO-1, PD-L1, PSCA, PSMA, ROR1, VEGFR2, BAFF-R, DLL-3 and SLAM-F7.

The antigen-binding capability of the CAR is defined by the extracellular scFv, not the targeted antigen. The format of a scFv is generally two variable domains linked by a flexible peptide sequence, either in the orientation VH-linker-VL or VL-linker-VH. The orientation of the variable domains within the scFv, depending on the structure of the scFv, may contribute to whether a CAR will be expressed on the ML NK cell surface or whether the CARML NK cells target the antigen and signal. In addition, the length and/or composition of the variable domain linker can contribute to the stability or affinity of the scFv.

scFvs are well known in the art to be used as a binding moiety in a variety of constructs (see e.g., Sentman 2014 Cancer J. 20 156-159; Guedan 2019 Mol Ther Methods Clin Dev. 12 145-156). Any scFv known in the art or generated against an antigen using means known in the art can be used as the binding moiety.

CAR scFv affinities, modified through mutagenesis of complementary-determining regions while holding the epitope constant, or through CAR development with scFvs derived from therapeutic antibodies against the same target, but not the same epitope, can change the strength of the ML NK cell signal and allow CAR NK cells to differentiate overexpressed antigens from normally expressed antigens. The scFv, a critical component of a CAR molecule, can be carefully designed and manipulated to influence specificity and differential targeting of tumors versus normal tissues. Given that these differences may only be measurable with CAR NK cells (as opposed to soluble antibodies), pre-clinical testing of normal tissues for expression of the target, and susceptibility to on-target toxicities, requires live-cell assays rather than immunohistochemistry on fixed tissues.

The scFvs described herein can be used for hematological malignancies such as AML, ALL, or Lymphoma, but can also be expanded for use in any malignancy, autoimmune, or infectious disease where a scFv can be generated against a target antigen or antigen epitope. For example, the constructs described herein can be used to treat or prevent autoimmunity associated with auto-antibodies (similar indications as rituximab for autoimmunity). As another example, the disclosed constructs can also be applied to virally infected cells, using scFv that can recognize viral antigens, for example gp120 and gp41 on HIV-infected cells. scFv sequences and specificities:

Anti-CD19 scFv
(SEQ ID NO: 1)
ATGGCCCT GCCCGTGACCGCTCTCCT GCTGCCTCTGGCCCT GCTCCTCCATGCT
GCCAG ACCCGACATCCAGAT GACACAGACAACCAGCAGCCT
GTCCGCTTCCCTCGGAGACAGGG TGACAATTTCCTGCAGGGCCAGCCAGGACAT
CAGCAAGTACCTGAACTGGTACCAGCAG AAACCCGACGGCACCGT CAAGCTCCT
GATCTACCACACCAGCAGACTGCACAGCGGAGT
GCCTTCCAGGTTCAGCGGCAGCGGCT CCGGCACCGATTACT CCCT
GACCATTAGCAACT TAGAACAGGAGGACATT
GCCACCTACTTTTGTCAGCAGGGCAACACCCTCCCCTACACC
TTTGGAGGCGGAACCAAGTTAGAAAT
CACCGGCGGCGGCGGCAGCGGAGGAGGAGGCAG
CGGAGGCGGAGGCTCCGAGGTGAAACTGCAGGAGAGCGGCCCCGGACTGGTCGCCC
CTA
GCCAATCCCTCTCCGTCACCTGCACCGTGAGCGGAGTGAGCCTGCCTGACTACGGAG
TG AGCT GGAT CAGACAGCCCCCTAGGAAAGGACTGGAATGGCT
GGGCGTGATTTGGGGCAG
CGAGACCACCTATTACAACAGCGCCCTGAAGTCCAGACTGACAAT
CATCAAGGACAATA GCAAAAGCCAAGT GTTT
CTGAAGATGAACAGCCTGCAGACCGATGACACCGCCATCTAT TATT
GCGCCAAGCACTACTACTACGGAGGAAGCTACGCTAT GGATTATTGGGGCCAAGG
CACAAGCGTGACCGTCAGCAGCGCGGCCGCC
Anti-CD33 scFv
(SEQ ID NO: 2)
ATGGCCTTACCAGTGACCGCCTTGCT CCTGCCGCTGGCCTT
GCTGCTCCACGCCGCCAG GCCGATGGAAAAGGATACACTGTTGTTGTGGGTTCT
GCTCCTGTGGGTGCCCGGCAGCA CCGGAGATATTGT GCTGACGCAGTCT
CCTGCATCACTCGCCGTGT CTCTGGGCCAGCGC GCTACCAT CAGCT GCAGAGCCT
CTGAAAGTGTTGACAATTATGGAATTTCTTTCAT GAA
TTGGTTCCAGCAGAAGCCTGGCCAGCCCCCGAAACT
CCTCATATATGCCGCGTCTAATC AGGGCTCT GGGGT CCCT
GCTAGATTTTCTGGCAGCGGCTCCGGCACCGACTTCAGTCTG
AATATACATCCCATGGAAGAAGACGATACCGCCATGTACTTTTGCCAACAATCTAAG
GA GGTGCCTT
GGACGTTCGGCGGCGGTACGAAGCTGGAAATTAAGGGCGGCGGGGGAAGCG
GCGGGGGGGGATCAGGCGGGGGTGGCTCCGGAGGCGGTGGAAGTATGGGCTGGAG
TTGG
ATCTTCCTTTTCCTTCTTTCTGGTACCGCGGGAGTGCACTCTGAGGTGCAGCTCCAGC
A GTCCGGCCCCGAGCTCGTCAAGCCTGGGGCCAGTGT
CAAGATTTCCTGTAAGGCATCTG GAT AT AC CT TT AC AGAT T AC AAT AT G CAT
TGGGT GAAAC AGT C AC AT GGAAAGT C ACT C GAGT GGAT
CGGATACATTTACCCTTACAATGGAGGAACCGGATATAATCAGAAGTTTAA
GAGCAAGGCCACACTCACGGTGGACAATTCTTCATCTACAGCCTACATGGATGTTCG
GT CTCT
GACTTCCGAGGATAGTGCGGTGTATTACTGCGCCAGGGGACGCCCCGCTATGGAT
TACT GGGGGCAGGGAACCTCTGTAACAGTTAGCTCA
Anti-CD123 scFv
(SEQ ID NO: 3)
ATGGCCTTACCAGTGACCGCCTTGCT CCTGCCGCTGGCCTT
GCTGCTCCACGCCGCCAG GCCGGACTTCGTGAT GACTCAGTCTCCTAGCTCCCT
GACCGTGACAGCCGGCGAGAAGG TGACCATGTCCTGCAAATCTAGTCAGAGTCT
GCTGAACTCAGGCAATCAGAAGAACTAT
CTGACATGGTACCTGCAGAAGCCAGGGCAGCCCCCTAAACT
GCTGATCTATTGGGCCAG
CACCAGGGAATCCGGCGTGCCCGACAGATTCACCGGCTCCGGGTCTGGAACAGATT
TTA CTCT GACCATTTCAAGCGTGCAGGCCGAGGACCTGGCTGTGTACTATTGT
CAGAAT GAT
TACAGCTATCCCTACACATTTGGCGGGGGAACTAAGCTGGAAATCAAAGGTGGT
GGTGG TTCT GGTGGTGGT GGTT
CCGGCGGCGGCGGCTCCGGTGGTGGTGGATCCGAGGT GCAGC
TGCAGCAGAGTGGACCCGAACT GGTGAAACCTGGCGCCTCCGTGAAAATGTCTT
GCAAG GCTAGTGGGTACACCTT CACAGACTACTATATGAAATGGGT
GAAGCAGTCACACGGGAA
GAGCCTGGAGTGGATCGGAGATATCATTCCCTCTAACGGCGCCACTTTCTACAATCA
GA AGTTTAAAGGCAAGGCTACTCT
GACCGTGGACCGGAGCTCCTCTACCGCCTATATGCAC
CTGAACAGTCTGACATCAGAAGATAGCGCTGTGTACTATTGTACACGGTCCCAT
CTGCT GAGAGCCT
CTTGGTTTGCTTATTGGGGCCAGGGGACACTGGTGACTGTGAGCTCCGCTA
GCACCACGACGCCAGCGCCGCGACCACCAACACCGGCGCCCACCATCGCGTCGCAG
CCC CTGT CCCT GCGCCCAGAGGCGT
GCCGGCCAGCGGCGGGGGGCGCAGTGCACACGAGGGG
GCTGGACTTCGCCTGTGATTTTTGGGTGCTGGTGGT
GGTTGGTGGAGTCCTGGCTTGCT
ATAGCTTGCTAGTAACAGTGGCCTTTATTATTTTCT GGGTG

Receptor and subunit sequences and specificities:

CD16
(SEQ ID NO: 44)
Atggccttaccagtgaccgccttgctcctgccgctggccttgctgctccacgccgccaggccgggcatgcgcaccgaggacctgcccaa
ggccgtggtgttcctggagccccagtggtaccgcgtgctggagaaggactccgtgaccctgaagtgccagggcgcctactcccccgagg
acaactccacccagtggttccacaacgagtccctgatctcctcccaggcctcctcctacttcatcgacgccgccaccgtggacgactccg
gcgagtaccgctgccagaccaacctgtccaccctgtccgaccccgtgcagctggaggtgcacatcggctggctgctgctgcaggcccccc
gctgggtgttcaaggaggaggaccccatccacctgcgctgccactcctggaagaacaccgccctgcacaaggtgacctacctgcagaacg
gcaagggccgcaagtacttccaccacaactccgacttctacatccccaaggccaccctgaaggactccggctcctacttctgccgcggcct
gttcggctccaagaacgtgtcctccgagaccgtgaacatcaccatcacccagggcctggccgtgcccaccatctcctccttcttccccccc
ggctaccag
CD64
(SEQ ID NO: 45)
CAGGTGGACACCACCAAGGCCGTGATCACCCTGCAGCCCCCCTGGGTGTCCGTGTTC
CAGGAGGAGACCGTGACCCTGCACTGCGAGGTGCTGCACCTGCCCGGCTCCTCCTCC
ACCCAGTGGTTCCTGAACGGCACCGCCACCCAGACCTCCACCCCCTCCTACCGCATC
ACCTCCGCCTCCGTGAACGACTCCGGCGAGTACCGCTGCCAGCGCGGCCTGTCCGGC
CGCTCCGACCCCATCCAGCTGGAGATCCACCGCGGCTGGCTGCTGCTGCAGGTGTCC
TCCCGCGTGTTCACCGAGGGCGAGCCCCTGGCCCTGCGCTGCCACGCCTGGAAGGA
CAAGCTGGTGTACAACGTGCTGTACTACCGCAACGGCAAGGCCTTCAAGTTCTTCCA
CTGGAACTCCAACCTGACCATCCTGAAGACCAACATCTCCCACAACGGCACCTACCA
CTGCTCCGGCATGGGCAAGCACCGCTACACCTCCGCCGGCATCTCCGTGACCGTGAA
GGAGCTGTTCCCCGCCCCCGTGCTGAACGCCTCCGTGACCTCCCCCCTGCTGGAGGG
CAACCTGGTGACCCTGTCCTGCGAGACCAAGCTGCTGCTGCAGCGCCCCGGCCTGCA
GCTGTACTTCTCCTTCTACATGGGCTCCAAGACCCTGCGCGGCCGCAACACCTCCTC
CGAGTACCAGATCCTGACCGCCCGCCGCGAGGACTCCGGCCTGTACTGGTGCGAGG
CCGCCACCGAGGACGGCAACGTGCTGAAGCGCTCCCCCGAGCTGGAGCTGCAGGTG
CTGGGCCTGCAGCTGCCCACCCCCGTGTGGTTCCAC
CD3E
(SEQ ID NO: 46)
ATGCAGTCCGGCACCCACTGGCGCGTGCTGGGCCTGTGCCTGCTGTCCGTGGGCGTG
TGGGGCCAGGACGGCAACGAGGAGATGGGCGGCATCACCCAGACCCCCTACAAGGT
GTCCATCTCCGGCACCACCGTGATCCTGACCTGCCCCCAGTACCCCGGCTCCGAGAT
CCTGTGGCAGCACAACGACAAGAACATCGGCGGCGACGAGGACGACAAGAACATC
GGCTCCGACGAGGACCACCTGTCCCTGAAGGAGTTCTCCGAGCTGGAGCAGTCCGG
CTACTACGTGTGCTACCCCCGCGGCTCCAAGCCCGAGGACGCCAACTTCTACCTGTA
CCTGCGCGCCCGCGTGTGCGAGAACTGCATGGAGATGGACGTGATGTCCGTGGCCA
CCATCGTGATCGTGGACATCTGCATCACCGGCGGCCTGCTGCTGCTGGTGTACTACT
GGTCCAAGAACCGCAAGGCCAAGGCCAAGCCCGTGACCCGCGGCGCCGGCGCCGGC
GGCCGCCAGCGCGGCCAGAACAAGGAGCGCCCCCCCCCCGTGCCCAACCCCGACTA
CGAGCCCATCCGCAAGGGCCAGCGCGACCTGTACTCCGGCCTGAACCAGCGCCGCA
TC
CD3G
(SEQ ID NO: 47)
ATGGAGCAGGGCAAGGGCCTGGCCGTGCTGATCCTGGCCATCATCCTGCTGCAGGG
CACCCTGGCCCAGTCCATCAAGGGCAACCACCTGGTGAAGGTGTACGACTACCAGG
AGGACGGCTCCGTGCTGCTGACCTGCGACGCCGAGGCCAAGAACATCACCTGGTTC
AAGGACGGCAAGATGATCGGCTTCCTGACCGAGGACAAGAAGAAGTGGAACCTGG
GCTCCAACGCCAAGGACCCCCGCGGCATGTACCAGTGCAAGGGCTCCCAGAACAAG
TCCAAGCCCCTGCAGGTGTACTACCGCATGTGCCAGAACTGCATCGAGCTGAACGCC
GCCACCATCTCCGGCTTCCTGTTCGCCGAGATCGTGTCCATCTTCGTGCTGGCCGTGG
GCGTGTACTTCATCGCCGGCCAGGACGGCGTGCGCCAGTCCCGCGCCTCCGACAAG
CAGACCCTGCTGCCCAACGACCAGCTGTACCAGCCCCTGAAGGACCGCGAGGACGA
CCAGTACTCCCACCTGCAGGGCAACCAGCTGCGCCGCAAC

### Illustrative Hinge Regions

The hinge, also referred to as a spacer, is in the extracellular structural region of the CAR that separates the binding units from the transmembrane domain. The hinge can be any moiety capable of ensuring proximity of the cell of interest to the target (e.g., NKG2-based hinge, TMa-based hinge, CD8− based hinge). With the exception of a few CARs based on the entire extracellular moiety of a receptor, such as NKG2D, as described herein, the majority of CAR (such as CAR T) cells are designed with immunoglobulin (Ig)-like domain hinges.

Hinges generally supply stability for efficient CAR expression and activity. The NKG2 hinge (also in combination with the transmembrane domain), described herein also ensures proper proximity to target.

The hinge also provides flexibility to access the targeted antigen. The optimal spacer length of a given CAR can depend on the position of the targeted epitope. Long spacers can provide extra flexibility to the CAR and allow for better access to membrane-proximal epitopes or complex glycosylated antigens. CARs bearing short hinges can be more effective at binding membrane-distal epitopes. The length of the spacer can be important to provide adequate intercellular distance for immunological synapse formation. As such, hinges may be optimized for individual epitopes accordingly. Illustrative hinge and TM sequences are provided below.

### Illustrative Transmembrane Domains

In some embodiments, the constructs described herein incorporate a transmembrane (TM) domain typically consisting of a hydrophobic a helix that spans the cell membrane. Illustrative TM domains include, for example, NKG2D, FcyRIIIa, NKp44, NKp30, NKp46, activating KIR, NKG2C, CD8a, CD3e, CD3g, or CD3d.

### Hinge/Transmembrane (TM) Domain Sequences

NKG2D (Hinge (SEQ ID NO: 4)/TM (SEQ ID NO: 5))
(SEQ ID NO: 4)
TCCACAAGAATCAAGATCTTCCCTCTCTGAGCAGGAATCCTTTGTGCATTGAAGACT
TTAGATTCCTCTCTGCGGTAGACGTGCACTTATAAGTATTTGATGGGGTGGATTCGT
GGTCGGAGGTCTCGACACAGCTGGGAGATGAGTGAATTTCATAATTATAACTTGGAT
CTGAAGAAGAGTGATTTTTCAACACGATGGCAAAAGCAAAGATGTCCAGTAGTCAA
AAGCAAATG TAGAGAAAATGCATCT;
(SEQ ID NO: 5)
CCATTTTTTTTCTGCTGCTTCATCGCTGTAGCCATGGGAATCCGTTTCATTATTATGGT
AACAATATGGAGT
FcyRIIIa (Hinge (SEQ ID NO: 6)/TM (SEQ ID NO: 7))
(SEQ ID NO: 6)
CACCTGAGGTGTCACAGCTGGAAGAACACTGCTCTGCATAAGGTCACATATTTACAG
AATGGCAAAGGCAGGAAGTATTTTCATCATAATTCTGACTTCTACATTCCAAAAGCC
ACACTCAAAGACAGCGGCTCCTACTTCTGCAGGGGGCTTTTTGGGAGTAAAAATGTG
TCTTCA GAGACT GT GAACAT CAC CAT CACT CAAGGTT T GGCAGT GT C AACC
AT CT C AT CATT CTT TCCACCTGGGTACCAA;
(SEQ ID NO: 7)
GTCTCTTTCTGCTTGGTGATGGTACTCCTTTTTGCAGTGGAC ACAGGACT AT ATT T
CT CT GT GAAGAC AAACA
NKp44 (Hinge (SEQ ID NO: 8)/TM (SEQ ID NO: 9))
(SEQ ID NO: 8)
TGTAGAATCTACCGCCCTTCTGACAACTCTGTCTCTAAGTCCGTCAGATTCTATCTGG
TGGTATCTCCAGCCTCTGCCTCCACACAGACCTCCTGGACTCCCCGCGACCTGGTCT
CTT CACAGACCCAGACCCAGAGCTGTGTGCCTCC
CACTGCAGGAGCCAGACAAGCCCCT GAG
TCTCCATCTACCATCCCTGTCCCTTCACAGCCACAGAACTCCACGCTCCGCCCTGGCC
C TGCAGCCCCCATTGCC;
(SEQ ID NO: 9)
CTGGTGCCTGTGTTCTGTGGACTCCTCGTAGCCAAGAGCCTG
GTGCTGTCAGCCCTGCTCGTCTGGTGGGGG
NKp30 (Hinge (SEQ ID NO: 10)/TM (SEQ ID NO: 11))
(SEQ ID NO: 10)
TCCGTCACGTGGTTCCGAGATGAGGTGGTTCCAGGGAAGGAGGTGAGGAATGGAAC
CCC
AGAGTTCAGGGGCCGCCTGGCCCCACTTGCTTCTTCCCGTTTCCTCCATGACCACCA
GG
CTGAGCTGCACATCCGGGACGTGCGAGGCCATGACGCCAGCATCTACGTGTGCAGA
GTG
GAGGTGCTGGGCCTTGGTGTCGGGACAGGGAATGGGACTCGGCTGGTGGTGGAGAA
AGA ACATCCTCAGCTAGGG;
(SEQ ID NO: 11)
GCTGGTACAGTCCTCCTCCTTCGGGCTGGATTCTATGCTGTC
AGCTTTCTCTCTGTGGCCGTGGGCAGCACC
NKp46 (Hinge (SEQ ID NO: 12)/TM (SEQ ID NO: 13))
(SEQ ID NO: 12)
TTCCCCCTGGGCCCTGTGACCACAGCCCACAGAGGGACATACCGATGTTTTGGCTCC
TA
TAACAACCATGCCTGGTCTTTCCCCAGTGAGCCAGTGAAGCTCCTGGTCACAGGCGA
CA
TTGAGAACACCAGCCTTGCACCTGAAGACCCCACCTTTCCTGCAGACACTTGGGGCA
CC
TACCTTTTAACCACAGAGACGGGACTCCAGAAAGACCATGCCCTCTGGGATCACACT
GC CCAGAATCTCCTTCGG;
(SEQ ID NO: 13)
ATGGGCCTGGCCTTTCTAGTCCTGGTGGCTCTAGTGTGGTTC
CTGGTTGAAGACTGGCTCAGCAGGAAGAGG
Activating KIR (KIR2DS4) (Hinge (SEQ ID NO: 14)/TM (SEQ ID NO: 15))
(SEQ ID NO: 14)
AGGGAAGGGGAGGCCCATGAACGTAGGCTCCCTGCAGTGCGCAGCATCAACGGAAC
ATT CCAGGCCGACTTT CCTCTGGGCCCTGCCACCCACGGAGGGACCTACAGAT
GCTT CGGCT CTTT CCGT GACGCTCCCTACGAGTGGTCAAACTCGAGTGAT
CCACTGCTT GTTT CCGTC ACAGGAAACCCTT CAAATAGTT GGCCTTCACCCACT
GAACCAAGCTCCAAAACCGGTAA CCCCAGACACCTACAT;
(SEQ ID NO: 15)
GTTCT GATT GGGACCTCAGTGGTCAAAATCCCTTTCACCATC CTCCTCTT
CTTTCTCCTTCATCGCTGG
NKG2C (Hinge (SEQ ID NO: 16)/TM (SEQ ID NO: 17))
(SEQ ID NO: 16)
AT GAATAAACAAAGAGGAACCTTCTCAGAAGTGAGT
CTGGCCCAGGACCCAAAGCGGCA
GCAAAGGAAACCTAAAGGCAATAAAAGCTCCATTTCAGGAACCGAACAGGAAATAT
TCC AAGTAGAATTAAATCTT CAAAATCCTTCCCT
GAATCATCAAGGGATTGATAAAATATAT GACT GCCAAGGTTTACT
GCCACCTCCAGAGAAG;
(SEQ ID NO: 17)
CT CACTGCCGAGGTCCTAGGAATCA TTTGCATT GTCCT
GATGGCCACTGTGTTAAAAACAATAGTT CTTATTCCTTTC
CD8a (Hinge (SEQ ID NO: 18)/TM (SEQ ID NO: 19))
(SEQ ID NO: 18)
GTCCTCACCCTGAGCGACTTCCGCCGAGAGAACGAGGGCTACTATTTCTGCTCGGCC
CTGAGCAACT CCATCATGTACTTCAGCCACTTCGTGCCGGTCTTCCT
GCCAGCGAAGCCCACCACGACGCCAGCGCCGCGACCACCAACACCGGCGCCCACCA
TCGCGTCGCAGCCCCTGTCCCTGCGCCCAGAGGCGTGCCGGCCAGCGGCGGGGGGC
GCAGTGCACACGAGGGGGCT
GGACTTCGCCTGT GAT;
(SEQ ID NO: 19)
ATCTACATCTGGGCGCCCTTGGCCGGGACTTGT
GGGGTCCTTCTCCTGTCACTGGTTAT CACCCTTTACTGC
IL15Rb (Hinge (SEQ ID NO: 20)/TM (SEQ ID NO: 21))
(SEQ ID NO: 20)
GCCT CCCACTACTTTGAAAGACACCT
GGAGTTCGAGGCCCGGACGCTGTCCCCAGGCCA
CACCTGGGAGGAGGCCCCCCTGCTGACTCTCAAGCAGAAGCAGGAATGGATCTGCC
TGG AGACGCTCACCCCAGACACCCAGTAT GAGTTTCAGGTGCGGGTCAAGCCT
CTGCAAGGC GAGTTCACGACCT GGAGCCCCT GGAGCCAGCCCCTGGCCTT
CAGGACAAAGCCT GCAGC CCTT GGGAAGGACACC;
(SEQ ID NO: 21)
ATTCCGTGGCTCGGCCACCTCCTCGTGGGCCTCAGCGGGGCT TTTGGCTT
CATCATCTTAGTGTACTT GCTGATCAACTGCAGG

### Illustrative Intracellular Signaling Domains (Costimulatory Domains)

In some embodiments, the CAR construct comprises one or more intracellular signaling domains. In some embodiments, the one or more intracellular signaling domains are active and effective in NK cells.

In some embodiments, an intracellular signaling domain can be any co-activating receptor capable of functioning in an NK cell (e.g., a ML NK cell). For example, a co-activating receptor can be CD137/41 BB (TRAF, NFkB), DNAM-1 (Y-motif), NKp80 (Y-motif), 2B4 (SLAMF)::ITSM, CRACC (CS1/SLAMF7)::ITSM, CD2 (Y-motifs, MAPK/Erk), CD27 (TRAF, NFkB), or integrins (e.g., multiple integrins).

In some embodiments, an intracellular signaling domain can be a cytokine receptor capable of functioning in an NK cell (e.g., a ML NK cell). For example, a cytokine receptor can be a cytokine receptor associated with persistence, survival, or metabolism, such as IL2R-beta or IL2R-gamma::Jak1/3, STAT3/5, PI3K/mTOR, MAPK/ERK. As another example, a cytokine receptor can be a cytokine receptor associated with activation, such as IL-18R::NFkB. As another example, a cytokine receptor can be a cytokine receptor associated with IFN-gamma production, such as IL-12R::STAT4. As another example, a cytokine receptor can be a cytokine receptor associated with cytotoxicity or persistence, such as IL-21R::Jak3/Tyk2, or STAT3.

In some embodiments, an intracellular signaling domain can be derived from a transmembrane protein with a signaling domain, such as FceR1 y (ITAMx1), CD3z (ITAMx3), DAP 12 (ITAMx1), or DAP 10 (YxxM/YINM).

In some embodiments, CAR intracellular signaling domains (also known as endodomains) can be derived from costimulatory molecules from the CD28 family (such as CD28 and ICOS) or the tumor necrosis factor receptor (TNFR) family of genes (such as 4-1 BB, 0X40, or CD27). The TNFR family members signal through recruitment of TRAF proteins and are associated with cellular activation, differentiation and survival.

In some embodiments, CD28 and 4-1 BB are used as costimulatory endodomains in CARs. In some embodiments, the high effector function and self-limited expansion of CD28-based CARs may be ideal to transiently treat diseases with a rapid tumor elimination and short-term persistence of the CAR in ML NK cells (i.e., as a bridge therapy for allogeneic hematopoietic stem cell transplantation). Furthermore, 4-1 BB-based CARs may be used to treat diseases in which complete response may require sustained NK cell persistence.

In some embodiments, an intracellular signaling domain can be an ITAM containing domain. In some embodiments, the ITAM containing domain can comprise CD79-alpha and CD79-beta.

In some embodiments, other domains, such as incorporation of ICOS can be used.

In some embodiments, a CAR cell can join the properties of different intracellular domains by combining two or more intracellular domains in a CAR. For example, such combinations can include one intracellular domain from the CD28 family and one intracellular domain from the TNFR family, resulting in the simultaneous activation of different signaling pathways.

In some embodiments, an illustrative costimulatory domain useful according to the present disclosure is a sequence such as those set out below.

CD137/41BB
(SEQ ID NO: 22)
aaacggggcagaaagaaactcctgtatatattcaaacaaccatttatgagaccagtacaaactactcaagaggaagatggctgtagctgccg
atttccagaagaagaagaaggaggatgtgaactg
DNAM-1
(SEQ ID NO: 23)
aaggagaaggagagagagaagagatctatttacagagtcctgggatacacagaaggcacccaataactatagaagtcccatctctaccagt
caacctaccaatcaatccatggatgatacaagagaggatatttatgtcaactatccaaccttctctcgcagaccaaagactagagt ttaag
NKp80
(SEQ ID NO: 24)
tttctcagggagtattgctaaaatgccaaaaaggaagttgttcaaatgccactcagtatgaggacactggagatctaaaagtgaataatggca
caagaagaaatataagtaataaggacctttgtgcttcgagatctgcagaccagacagtactatgccaatcagaatggctcaaataccaaggg
aagtgttattggttctctaatgagatgaaaagctggagtgacagttatgtgtattgtttggaaagaaaatctcatctactaatcatacatgac
caacttgaaatggcttttatacagaaaaacctaagacaattaaactacgtatggattgggcttaactttacctccttgaaaatgacatggact
tgggtggatggttctccaatagattcaaagatattcttcataaagggaccagctaaagaaaacagctgtgctgccattaaggaaagcaaaatt
ttctctgaaacctgcagcagtgttttcaaatggatttgtcagtattag
2B4
(SEQ ID NO: 25)
agaccagtcccaaggaatttttgacaatttacgaagatgtcaaggatctgaaaaccaggagaaatcacgagcaggagcagacttttcctgga
ggggggagcaccatctactctatgatccagtcccagtcttctgctcccacgtcacaagaacctgcatatacattatattcattaattcagcct
tccaggaagtctggatccaggaagaggaaccacagcccttccttcaatagcactatctatgaagtg
NTBA
(SEQ ID NO: 26)
attccctatctttgtctactcagcgaacacagggccccgcagagtccgcaaggaacctagagtatgtttcagtgtctccaacgaacaacactg
tgtatgcttcagtcactcattcaaacagggaaacagaaatctggacacctagagaaaatgatactatcacaatttactccacaattaatcatt
ccaaagagagtaaacccactttttccagggcaactgcccttgacaatgtc gtgtaa
CRACC
(SEQ ID NO: 27)
agtacattgaagagaagaagagagtggacatttgtcgggaaactcctaacatatgcccccattctggagagaacacagagtacgacacaat
ccctcacactaatagaacaatcctaaaggaagatccagcaaatacggtttactccactgtggaaataccgaaaaag
CD2
(SEQ ID NO: 28)
aaaaggaaaaaacagaggagtcggagaaatgatgaggagctggagacaagagcccacagagtagctactgaagaaaggggccggaa
gccccaccaaattccagcttcaacccctcagaatccagcaacttcccaacatcctcctccaccacctggtcatcgttcccaggcacctagtca
tcgtcccccgcctcctggacaccgtgttcagcaccagcctcagaagaggcctcctgctccgtcgggcacacaagttcaccagcagaaagg
cccgcccctccccagacctcgagttcagccaaaacctccccatggggcagcagaaaactcattgtccccttcctctaattaa
CD27
(SEQ ID NO: 29)
atccttgtgatcttctctggaatgttccttgttttcaccctggccggggccctgttcctccatcaacgaaggaaatatagatcaaacaaagga
gaaagtcctgtggagcctgcagagccttgtcgttacagctgccccagggaggaggagggcagcaccatccccatccaggaggattaccgaa
aaccggagcctgcctgctccccctga
Integrins
ITGB1
(SEQ ID NO: 30)
aagcttttaatgataattcatgacagaagggagtttgctaaatttgaaaaggagaaaatgaatgccaaatgggacacgggtgaaaatcctattt
ataagagtgccgtaacaactgtggtcaatccgaagtatgagggaaaatga
ITGB2
(SEQ ID NO: 31)
aaggctctgatccacctgagcgacctccgggagtacaggcgctttgagaaggagaagctcaagtcccagtggaacaatgataatccccttt
tcaagagcgccaccacgacggtcatgaaccccaagtttgctgagagttag
ITGB3
(SEQ ID NO: 32)
aaactcctcatcaccatccacgaccgaaaagaattcgctaaatttgaggaagaacgcgccagagcaaaatgggacacagccaacaaccc
actgtataaagaggccacgtctaccttcaccaatatcacgtaccggggcacttaa
IL15RB
(SEQ ID NO: 33)
aactgcaggaacaccgggccatggctgaagaaggtcctgaagtgtaacaccccagacccctcgaagttcttttcccagctgagctcagag
catggaggagacgtccagaagtggctctcttcgcccttcccctcatcgtccttcagccctggggcctggcacctgagatctcgccactaga
agtgctggagagggacaaggtgacgcagctgctcctgcagcaggacaaggtgcctgagcccgcatccttaagcagcaaccactcgctga
ccagctgcttcaccaaccagggttacttcttcttccacctcccggatgccttggagatagaggcctgccaggtgtactttacttacgacccct
actcagaggaagaccctgatgagggtgtggccggggcacccacagggtc
ttccccccaacccctgcagcctctgtcaggggaggacgacgcctactgcaccttcccctccagggatgacctgctgctcttctcccccagtc
tcctcggtggccccagccccccaagcactgcccctgggggcagtggggccggtgaagagaggatgcccccttctttgcaagaaagagtc
cccagagactgggacccccagcccctggggcctcccaccccaggagtcccagacctggtggattttcagccaccccctgagctggtgct
gcgagaggctggggaggaggtccctgacgctggccccagggagggagtcagtttcccctggtccaggcctcctgggcagggggagttc
agggcccttaatgctcgcctgcccctgaacactgatgcctacttgtccctccaagaactccagggtcaggacccaactcacttggtgtag
IL18R
(SEQ ID NO: 34)
tataaagttgacttggttctgttctataggcgcatagcggaaagagacgagacactaacagatggtaaaacatatgatgcctttgtgtcttac
ctgaaagagtgtcatcctgagaataaagaagagtatacttttgctgtggagacgttacccagggtcctggagaaacagtttgggtataagtta
tgcatatttgaaagagatgtggtgcctggcggagctgttgtcgaggagatccattcactgatagagaaaagccggaggctaatcatcgttctc
agccagagttacctgactaacggagccaggcgtgagctcgagagtggactccacgaagcactggtagagaggaagattaagatcatcttaa
ttgagtttactccagccagcaacatcacctttctccccccgtcgctgaaactcctgaagtcctacagagttctaaaatggagggctgacagtc
cctccatgaactcaaggttctggaagaatcttgtttacctgatgcccgcaaaagccgtcaagccatggagagaggagtcggaggcgcggtct
gttctctcagcaccttga
IL12R
IL12RB1
(SEQ ID NO: 35)
aacagggccgcacggcacctgtgcccgccgctgcccacaccctgtgccagctccgccattgagttccctggagggaaggagacttggca
gtggatcaacccagtggacttccaggaagaggcatccctgcaggaggccctggtggtagagatgtcctgggacaaaggcgagaggact
gagcctctcgagaagacagagctacctgagggtgcccctgagctggccctggatacagagttgtccttggaggatggagacaggtgcaa
ggccaagatgtga
IL12RB2
(SEQ ID NO: 36)
cattacttccagcaaaaggtgtttgttctcctagcagccctcagacctcagtggtgtagcagagaaattccagatccagcaaatagcacttgc
gctaagaaatatcccattgcagaggagaagacacagctgcccttggacaggctcctgatagactggcccacgcctgaagatcctgaaccg
ctggtcatcagtgaagtccttcatcaagtgaccccagttttcagacatcccccctgctccaactggccacaaagggaaaaaggaatccaagg
tcatcaggcctctgagaaagacatgatgcacagtgcctcaagcccaccacctccaagagctctccaagctgagagcagacaactggtgga
tctgtacaaggtgctggagagcaggggctccgacccaaagcccgaaaacccagcctgtccctggacggtgctcccagcaggtgaccttc
ccacccatgatggctacttaccctccaacatagatgacctcccctcacatgaggcacctctcgctgactctctggaagaactggagcctcag
cacatctccctttctgttttcccctcaagttctcttcacccactcaccttctcctgtggtgataagctgactctggatcagttaaagatgag
gtgtgactccctcatgctctga
IL21R
(SEQ ID NO: 37)
agcctgaagacccatccattgtggaggctatggaagaagatatgggccgtccccagccctgagcggttcttcatgcccctgtacaagggct
gcagcggagacttcaagaaatggggggtgcacccttcactggctccagcctggagctgggaccctggagcccagaggtgccctccacc
ctggaggtgtacagctgccacccaccacggagcccggccaagaggctgcagctcacggagctacaagaaccagcagagctggtggag
tctgacggtgtgcccaagcccagcttctggccgacagcccagaactcggggggctcagcttacagtgaggagagggatcggccatacgg
cctggtgtccattgacacagtgactgtgctagatgcagaggggccatgcacctggccctgcagctgtgaggatgacggctacccagccct
ggacctggatgctggcctggagcccagcccaggcctagaggacccactcttggatgcagggaccacagtcctgtcctgtggctgtgtctc
agctggcagccctgggctaggagggcccctgggaagcctcctggacagactaaagccaccccttgcagatggggaggactgggctggg
ggactgccctggggtggccggtcacctggaggggtctcagagagtgaggcgggctcacccctggccggcctggatatggacacgtttga
cagtggctttgtgggctctgactgcagcagccctgtggagtgtgacttcaccagccccggggacgaaggacccccccggagctacctccg
ccagtgggtggtcattcctccgccactttcgagccctggaccccaggccagctaa
IRE1a
(SEQ ID NO: 38)
cccctgagcatgcatcagcagcagcagctccagcaccagcagttccagaaggaactgga
gaagatccagctcctgcagcagcagcagcagcagctgcccttccacccacctggagacacggctcaggacggcgagctcctggacacg
tctggcccgtactcagagagctcgggcaccagcagccccagcacgtcccccagggcctccaaccactcgctctgctccggcagctctgc
ctccaaggctggcagcagcccctccctggaacaagacgatggagatgaggaaaccagcgtggtgatagttgggaaaatttccttctgtccc
aaggatgtcctgggccatggagctgagggcacaattgtgtaccggggcatgtttgacaaccgcgacgtggccgtgaagaggatcctcccc
gagtgttttagcttcgcagaccgtgaggtccagctgttgcgagaatcggatgagc
acccgaacgtgatccgctacttctgcacggagaaggaccggcaattccagtacattgccatcgagctgtgtgcagccaccctgcaagagta
tgtggagcagaaggactttgcgcatctcggcctggagcccatcaccttgctgcagcagaccacctgggcctggcccacctccactccctc
aacatcgttcacagagacctaaagccacacaacatcctcatatccatgcccaatgcacacggcaagatcaaggccatgatctccgactttgg
cctctgcaagaagctggcagtgggcagacacagtttcagccgccgatctggggtgcctggcacagaaggctggatcgctccagagatgct
gagcgaagactgtaaggagaaccctacctacacggtggacatcttttctgcaggctgcgtcttttactacgtaatctctgagggcagccaccc
ttttggcaagtccctgcagcggcaggccaacatcctcctgggtgcctgcagccttgactgcttgcacccagagaagcacgaagacgtcatt
gcacgtgaattgatagagaagatgattgcgatggatcctcagaaacgcccctcagcgaagcatgtgctcaaacacccgttcttctggagcct
agagaagcagctccagttcttccaggacgtgagcgacagaatagaaaaggaatccctggatggcccgatcgtgaagcagttagagagag
gcgggagagccgtggtgaagatggactggcgggagaacatcactgtccccctccagacagacctgcgtaaattcaggacctataaaggt
ggttctgtcagagatctcctccgagccatgagaaataagaagcaccactaccgggagctgcctgcagaggtgcgggagacgctggggtc
cctccccgacgacttcgtgtgctacttcacatctcgcttcccccacctcctcgcacacacctaccgggccatggagctgtgcagccacgaga
gactcttccagccctactacttccacgagcccccagagccccagcccccagtgactccagacgccctctga
CD79A
(SEQ ID NO: 48)
cgcaagcgctggcagaacgagaagctgggcctggacgccggcgacgagtacgaggacgagaacctgtacgagggcctgaacctgga
cgactgctccatgtacgaggacatctcccgcggcctgcagggcacctaccaggacgtgggctccctgaacatcggcgacgtgcagctgg
agaagccc
CD79B
(SEQ ID NO: 49)
ctggacaaggacgactccaaggccggcatggaggaggaccacacctacgagggcctggacatcgaccagaccgccacctacgaggac
atcgtgaccctgcgcaccggcgaggtgaagtggtccgtgggcgagcaccccggccaggag

Optionally, an extracellular signaling domain can be incorporated into the CAR construct to propagate signaling. The extracellular signaling domain can be cloned into the hinge region, such as a CD8 hinge, but can be chosen based on the target.

### Methods of Treatment and Other Uses

Methods of treatment are provided that comprise administration of hypoimmunogenic cells of the present disclosure or compositions comprising the same. Cell transplantation is one of the most promising therapeutic approaches for the treatment of intractable medical conditions in which cell replacement or ongoing support is required. Because the cells genetically engineered as disclosed herein have reduced immunogenicity, these cells are useful in the treatment of any of a number of indications.

Typically, the hypoimmunogenic cells described herein (e.g., somatic cells, hypoimmunogenic iPSCs or somatic cells derived therefrom) can effectively survive in a host without stimulating the host immune response for a desired duration, such as for at least one week, two weeks, one month, two months, three months, six months, one year, two years, three years, four years, five years or more, e.g., for the life of the cell and/or its progeny).

The methods of treatment that are carried out according to the present disclosure may be directed at treating the cause of the disease or condition; or alternatively, the therapy may be to treat the effects of the disease or condition. The hypoimmunogenic cells may be administered to, or close to, an injured site in a subject; or the cells can be administered to the subject in a manner allowing the cells to migrate, or home, to the injured site. In some instances, the administered cells may advantageously replace the damaged or injured cells and allow improvement in the overall condition of the subject. In some instances, the administered cells may advantageously support the viability of existing endogenous cells. In some instances, the administered cells may stimulate tissue regeneration or repair, including skin regeneration or skin repair.

Therefore, in some instances, the present disclosure provides methods of treating a disease or condition comprising administering to a subject in need a hypoimmunogenic cell or a population thereof that has been modified as described herein. This could be, for example, a hypoimmunogenic somatic cell, a hypoimmunogenic somatic cell derivative that has been differentiated from a hypoimmunogenic stem cell ex vivo or that has been transdifferentiated from a hypoimmunogenic somatic cell of a different type ex vivo.

In some embodiments of the disclosure, for example, the hypoimmunogenic cells administered to a subject comprise somatic cells, such as a hypoimmunogenic cardiomyocyte, a hypoimmunogenic retinal pigment epithelial cell, a hypoimmunogenic photoreceptor cell, a hypoimmunogenic neural cell, a hypoimmunogenic glial cell, a hypoimmunogenic hepatocyte, a hypoimmunogenic muscle cell, a hypoimmunogenic cartilage cell, a hypoimmunogenic chondrocyte, a hypoimmunogenic keratinocyte, a hypoimmunogenic beta islet cell, a hypoimmunogenic hepatocyte, a hypoimmunogenic parathyroid cell, a hypoimmunogenic thymic epithelial cell, a hypoimmunogenic endothelial cell, a hypoimmunogenic mesenchymal cell, a hypoimmunogenic CD34+ hematopoietic stem cell, or a hypoimmunogenic peripheral blood mononuclear cell (PBMC).

In other embodiments of the disclosure, the hypoimmunogenic cells administered to a subject comprise hypoimmunogenic immune cells, including hypoimmunogenic T cells (including cytotoxic T-cells, helper T-cells, memory T-cells, regulatory T-cells), hypoimmunogenic NK cells, hypoimmunogenic memory like NK cells, hypoimmunogenic macrophages, hypoimmunogenic tumor infiltrating lymphocytes, etc., as well as hypoimmunogenic CAR-T cells, hypoimmunogenic chimeric T cells (TCR-T cells), and the like, including progeny thereof.

In some embodiments of the disclosure, where somatic cell derivatives of hypoimmunogenic stem cells are used, the somatic cell derivatives can be essentially any cell type of interest. Thus, for example, the methods of the invention can comprise a step of differentiating hypoimmunogenic pluripotent stem cells into hypoimmunogenic cardiomyocytes, into hypoimmunogenic skeletal muscle, into hypoimmunogenic endothelial cells, into hypoimmunogenic neurons, into hypoimmunogenic retinal cells, e.g. RPE or photoreceptors, into hypoimmunogenic cartilage, into hypoimmunogenic hepatocytes, into hypoimmunogenic beta-like pancreatic cells or islet organoids, into hypoimmunogenic epithelial cells, or into hypoimmunogenic thymic epithelial progenitors, etc.

In further embodiments, the methods of the invention can comprise a step of differentiating hypoimmunogenic pluripotent stem cells into hypoimmunogenic immune cells, including hypoimmunogenic T cells (including cytotoxic T-cells, helper T-cells, memory T-cells, regulatory T-cells), NK cells, macrophages, tumor infiltrating lymphocytes, etc., as well as into hypoimmunogenic CAR-T cells, hypoimmunogenic chimeric T cells (TCR-T cells), and the like, including progeny thereof.

In some embodiments, the hypoimmunogenic somatic cell derivative used in the methods of the invention is a somatic cell that has been differentiated from an induced pluripotent stem cell while it is undergoing reprogramming. Put another way, the pluripotent cell is induced to reprogram and differentiate at the same time.

The methods of the invention include methods for the treatment of many diseases and conditions using appropriate types of hypoimmunogenic cells. In some embodiments, the disease or condition being treated is selected from the group consisting of cancer, an immune disorder (e.g., an autoimmune disease), blindness, spinal cord injury, ALS, Parkinson's disease, Huntington's disease, Alzheimer's disease, an enteric neuropathy, multiple sclerosis, osteoarthritis or joint injury, a disease of the skin such as Epidermolysis Bullosa, diabetes, liver disease, osteoporosis, kidney disease and DiGeorge Syndrome.

In some more particular embodiments, the disease or condition to be treated is cancer or an immune disorder and the hypoimmunogenic cells administered to the subject are hypoimmunogenic leukocytes, e.g.., T cells, NK cells, cytokine-induced memory-like (CIML) NK cells, and the like.

In some embodiments, the disease or condition to be treated is cancer or an immune disorder and the cells administered are stem cells (e.g., iPSCs) differentiated into leukocytes, e.g., T cells, NK cells, and the like, prior to their administration.

In some embodiments, the disease or condition to be treated an autoimmune disease and the cells administered are differentiated into Treg cells prior to their administration.

In some embodiments, the disease or condition to be treated is myocardial infarction and the cells administered to the subject in need are differentiated into cardiomyocytes prior to their administration.

In some embodiments, the disease or condition to be treated is blindness and the cells administered are differentiated into retinal pigment epithelial cells or photoreceptor cells prior to their administration.

In some embodiments, the disease or condition to be treated is spinal cord injury, ALS, Parkinson's disease, Huntington's disease, or Alzheimer's disease and the cells administered are differentiated into neurons or glia prior to their administration.

In some embodiments, the disease or condition to be treated is enteric neuropathy, and the cells administered are differentiated into enteric neurons prior to their administration.

In some embodiments, the disease or condition to be treated is multiple sclerosis and the cells administered are differentiated into glial cells prior to their administration.

In some embodiments, the disease or condition to be treated is osteoarthritis or joint injury and the cells administered are differentiated into chondrocytes or cartilage prior to their administration.

In some embodiments, the disease or condition to be treated is a disease of the skin such as Epidermolysis Bullosa and the cells administered are differentiated into keratinocytes prior to their administration.

In some embodiments, the disease or condition to be treated is diabetes and the cells administered are differentiated into pancreatic progenitors or endocrine cells prior to their administration.

In some embodiments, the disease or condition to be treated is liver disease and the cells administered are differentiated into hepatocytes prior to their administration.

In some embodiments, the disease or condition to be treated is osteoporosis and the cells administered are differentiated into parathyroid cells prior to their administration.

In some embodiments, the disease or condition to be treated is DiGeorge Syndrome and the cells administered are differentiated into thymic epithelial cells prior to their administration.

In some embodiments, the disease or condition to be treated is kidney disease and the cells administered are differentiated into the nephrons prior to their administration.

See, e.g., Human Embryonic and Induced Pluripotent Stem Cells, Lineage-Specific Differentiation Protocols, Editors: Ye, Kaiming, Jin, Sha (Eds.) Springer 2011; Human Embryonic Stem Cell Protocols. Methods in Molecular Biology, Turksen, Kursad (Ed.) 2016; Induced Pluripotent Stem (iPS) Cells: Methods and Protocols (Methods in Molecular Biology), by Kursad Turksen and Andras Nagy, Dec. 29, 2015, for illustrative and nonlimiting methods for differentiating somatic cells from pluripotent stem cells.

In still other instances, hypoimmunogenic pluripotent stem cells can be differentiated into an organ or tissue in vitro using methods known by those of skill in the art and administered to a subject in need of an organ or tissue transplant.

Alternatively, in other instances, the composition administered to a subject can include a population of hypoimmunogenic pluripotent stem cells with one or more factors that stimulate cell differentiation into a desired cell type, where the cell differentiation or maturation occurs in vivo at the tissue site.

Of course, it will be understood that the cell populations obtained and/or used according to the present disclosure may also have other modifications of interest. For example, they may express one or more desired growth factors or cytokines and/or they may have other modifications of interest.

The hypoimmunogenic cells and compositions described herein may be administered to a subject in need thereof (e.g., a subject who is receiving or has received a transplant, or a subject having a disease or condition described herein) by a variety of routes, such as local administration to or near the site of a transplant, local administration to the site affected by the disease or condition (e.g. injection to a joint for treating RA, injection into the subretinal space for treating AMD, direct administration to the central nervous system (CNS) (e.g., intracerebral, intraventricular, intrathecal, intracisternal, or stereotactic administration) for treating a neurological disease, such as Parkinson's disease, direct injection into the cardiac muscle for treating cardiac infarction), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular cells or composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, or monthly). For local administration, the hypoimmunogenic cells may be administered by any means that places the population of cells in a desired location, including catheter, syringe, shunt, stent, microcatheter, pump, implantation with a device, or implantation with a scaffold.

Subjects that may be treated as described herein include subjects that have received a transplant, or subjects having a disease or condition for which a regenerative therapy finds use (e.g., AMD or retinal dystrophy, a neurodegenerative disease, such as Parkinson's disease, cardiac infarction, osteoarthritis or RA, diabetes, hemophilia, a metabolic disorder, a skin disease such as Epidermolysis Bullosa, etc.). Put another way, a regenerative therapy encompassing transplantation of the hypoimmunogenic cells of the present disclosure finds use in any disease or condition caused by or associated with loss of cells, a mutation or deficiency in a protein, aberrant production of a protein, or injury which could be treated using cell replacement protein or cellular therapy, production of a therapeutic protein, production of an agonist antibody, or production of an inhibitory antibody. The methods described herein may include a step of screening a subject for mutations in genes associated with deficient protein production prior to treatment with or administration of the compositions described herein. A subject can be screened for a genetic mutation using standard methods known to those of skill in the art (e.g., genetic testing). The methods described herein may also include a step of evaluating the symptoms of the disease or condition in a subject prior to treatment with or administration of the hypoimmunogenic cells or compositions described herein. The subject can then be evaluated using the same diagnostic tests after administration of the hypoimmunogenic cells or compositions to determine whether the subject's condition has improved. The compositions and methods described herein may be administered as a preventative treatment to patients who have received a tissue or organ transplant before the patient shows any signs of tissue or organ rejection.

The hypoimmunogenic cells, compositions, and methods described herein can be used to replace dead or dying cells in a subject (e.g., to replace neurons in a subject suffering from a neurodegenerative disease, or to replace cardiac muscle cells in a subject who has had a myocardial infarction).

Hypoimmunogenic cells that express a therapeutic agent, such as a protein or agonist antibody, compositions including such cells, or methods of administering such cells, may be used to replace or supply wild type versions of proteins that are mutated or deficient in a subject (e.g., proteins that are produced but do not function correctly due to a genetic mutation, such as truncated proteins or proteins with altered charge, polarity, or binding properties); or proteins that are not produced or that are produced in insufficient quantities.

Hypoimmunogenic cells that express a therapeutic agent, such as an inhibitory or neutralizing antibody, compositions including such cells, or methods of administering such cells, may be used to block or neutralize proteins that are overexpressed in a subject or proteins that are aberrantly produced (e.g., proteins that are produced at a time or in a location that differs from production of that protein in healthy subjects, e.g., aberrant protein production that is associated with a disease or condition).

Treatment may include administration of hypoimmunogenic cells (e.g., hypoimmunogenic somatic cells, iPSCs or somatic cells derived therefrom) or a composition containing hypoimmunogenic cells in various unit doses. Each unit dose will ordinarily contain a predetermined quantity of the hypoimmunogenic cells described herein. The quantity to be administered, and the particular route of administration and formulation, are within the skill of those in the clinical arts. A unit dose need not be administered as a single injection but may comprise continuous infusion over a set period of time. Administration may be performed using a catheter, syringe, shunt, stent, microcatheter, pump, implantation with a device, or implantation with a scaffold. The number of cells administered may vary depending on whether the cells are administered to a tissue, organ, or body site associated with a disease or injury, or are administered subcutaneously to produce a hypoimmunogenic subcutaneous tissue.

For administration to a tissue, organ, or body site, the hypoimmunogenic cells may be administered to the patient at a dose of, for example 1×10\hat{ }4 cells to 1×10\hat{ }10 cells (e.g., 1×10\hat{ }4, 2×10\hat{ }4, 3×10\hat{ }4,4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10 cells). The number of cells administered will depend on the size of the recipient tissue, organ, or body site. For example, 1×10\hat{ }4 to 1×10\hat{ }8 cells (e.g., 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, or 1×10\hat{ }8 cells) can be administered (e.g., injected) to the subretinal space of the eye, to a specific brain region, or to a joint, with the quantity of cells depending on the size of the joint; and 1×10\hat{ }6 to 5×10\hat{ }9 cells (e.g., 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, or 1×10\hat{ }8 cells, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, or 5×10\hat{ }9 cells) can be administered to the cardiac muscle, e.g., via a patch or via direct injection into the heart muscle wall, for example. For creating hypoimmunogenic subcutaneous tissue, 8×10\hat{ }8 cells to 3×10\hat{ }9 cells (e.g., 8×10\hat{ }8, 9×10\hat{ }8, 1×10\hat{ }9, 2×10\hat{ }9, 3×10\hat{ }9 cells) can be administered (e.g., injected) subcutaneously.

Hypoimmunogenic cells can be administered in two or more doses (e.g., two, three, four, five, or more different doses, e.g., to joints of different sizes in a patient with RA) or at the same dose two or more times (e.g., two, three, four, five, six, or more times over the course of an hour, day, week, month, or year). In some embodiments, the hypoimmunogenic cells described herein are administered as a tissue (e.g., a tissue that has been grown and/or differentiated in vitro from hypoimmunogenic cells). In some embodiments, the hypoimmunogenic tissue is administered (e.g., implanted) with a gel, biocompatible matrix, or scaffold.

The compositions described herein are administered in an amount sufficient to improve symptoms of a disease or condition (e.g., to reduce the symptoms of cancer, to reduce symptoms of osteoarthritis or RA (e.g., reduce inflammation, joint pain, stiffness, or immobility); reduce symptoms of retinal dystrophy or wet AMD (e.g., improve vision, slow or reduce vascularization of the eye); reduce symptoms of Parkinson's disease (e.g., reduce tremors, rigidity, bradykinesia, or improve posture or gait); reduce symptoms of diabetes (e.g., improve insulin levels, reduce the need for regular insulin injections); reduce symptoms of cardiac infarction (e.g., improve heart function, reduce infarct size); reduce symptoms of hemophilia (e.g., increase level of blood coagulation factors, such as Factor VIII, reduce excessive bleeding, reduce bruising, reduce nosebleeds, reduce joint pain or swelling); or reduce symptoms of metabolic disorders (e.g., increase appetite, growth, or weight gain, or reduce lethargy, weight loss, jaundice, seizures, abdominal pain, or vomiting)).

Transplant rejection may be evaluated using standard methods known by those of skill in the art and may be reduced by 5% or more (e.g., 5%, 10%, 15%, 20%, 30%, 40%, 50%, 60%, 70%, 80%, 90%, 100%) compared to rates of transplant rejection typically observed without treatment. In some embodiments, administration of the hypoimmunogenic cells or compositions described herein results in an equivalent outcome in transplant rejection as that observed in subjects administered immunosuppressive agent(s).

Symptoms of diseases and conditions described herein can be evaluated using standard methods known to those of skill in the art and may be reduced (e.g., the subject's condition may be improved) by 5% or more (e.g., 5%, 10%, 15%, 20%, 30%, 40%, 50%, 60%, 70%, 80%, 90%, 100%) compared to symptoms prior to administration of the hypoimmunogenic cells or compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the hypoimmunogenic cell or composition depending on the dose and route of administration used for treatment.

### Pharmaceutical Compositions

The hypoimmunogenic cells described herein may be incorporated into a pharmaceutical composition for administration into a patient, such as a human patient receiving a transplant or suffering from a disease or condition described herein. Pharmaceutical compositions containing hypoimmunogenic cells can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of aqueous solutions.

The hypoimmunogenic cells described herein can be administered in any physiologically compatible carrier, such as a buffered saline solution. Pharmaceutically acceptable carriers and diluents include saline, aqueous buffer solutions, solvents and/or dispersion media. The use of such carriers and diluents is well known in the art. Other examples include liquid media, for example, Dulbeccos modified eagle's medium (DMEM), sterile saline, sterile phosphate buffered saline, Leibovitz's medium (L15, Invitrogen, Carlsbad, Calif.), dextrose in sterile water, and any other physiologically acceptable liquid. Dispersions may also be prepared in glycerol, liquid polyethylene glycols, and mixtures thereof and in oils. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. In many cases, it will be preferable to include isotonic agents, for example, sugars or sodium chloride. The solution is preferably sterile and fluid to the extent that easy syringeability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosol, and the like. Solutions of the invention can be prepared by using a pharmaceutically acceptable carrier or diluent and, as required, other ingredients enumerated above, followed by filtered sterilization, and then incorporating the hypoimmunogenic cells as described herein.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, intraperitoneal, intraventricular, subretinal, and intravitreal administration. In this connection, sterile aqueous media that can be employed will be known to those of skill in the art in light of the present disclosure. The person responsible for administration will, in any event, determine the appropriate dose for the individual subject. Moreover, for human administration, preparations may meet sterility, pyrogenicity, general safety, and purity standards as required by FDA Office of Biologics standards.

Pharmaceutical compositions comprising hypoimmunogenic cells in a semi-solid or solid carrier are typically formulated for surgical implantation at the site of transplantation or at the affected site of a disease or condition in the subject. It will be appreciated that liquid compositions also may be administered by surgical procedures. In particular embodiments, semi-solid or solid pharmaceutical compositions may comprise semi-permeable gels, matrices, cellular scaffolds and the like, which may be non-biodegradable or biodegradable. For example, in certain embodiments, it may be desirable or appropriate to sequester the hypoimmunogenic cells from their surroundings yet enable the cells to secrete and deliver biological molecules (e.g., a therapeutic agent) to surrounding cells.

In other embodiments, different varieties of degradable gels and networks are utilized for the pharmaceutical compositions of the invention. For example, degradable materials include biocompatible polymers, such as poly(lactic acid), poly(lactic acid-co-glycolic acid), methylcellulose, hyaluronic acid, collagen, and the like.

In another embodiment, one or more hydrogels are used for the pharmaceutical compositions. The one or more hydrogels may include collagen, atelocollagen, fibrin constructs, hydrophilic vinyl and acrylic polymers, polysaccharides such as calcium alginate, and poly(ethylene oxide). Further, the hydrogel may be formed of poly(2-hydroxyethyl methacrylate), poly(acrylic acid), self-assembling peptides (e.g., RAD16), poly(methacrylic acid), poly(N-vinyl-2-pyrrolidinone), polyvinyl alcohol) and their copolymers with each other and with hydrophobic monomers such as methyl methacrylate, vinyl acetate, and the like. Also preferred are hydrophilic polyurethanes containing large poly(ethylene oxide) blocks. Other preferred materials include hydrogels comprising interpenetrating networks of polymers, which may be formed by addition or by condensation polymerization, the components of which may comprise hydrophilic and hydrophobic monomers such as those just enumerated. In situ-forming degradable networks are also suitable for use in the invention (see, e.g., Anseth, K S et al. J. Controlled Release, 2002; 78: 199-209; Wang, D. et al., Biomaterials, 2003; 24:3969-3980; U.S. Patent Publication 2002/0022676). These in situ forming materials are formulated as fluids suitable for injection; then may be induced to form a hydrogel by a variety of means such as change in temperature, pH, and exposure to light in situ or in vivo. In one embodiment, the construct contains fibrin glue containing gels. In another embodiment, the construct contains atelocollagen containing gels.

A polymer used to form a matrix may be in the form of a hydrogel. In general, hydrogels are cross-linked polymeric materials that can absorb more than 20% of their weight in water while maintaining a distinct three-dimensional structure. This definition includes dry cross-linked polymers that will swell in aqueous environments, as well as water-swollen materials. A host of hydrophilic polymers can be cross-linked to produce hydrogels, whether the polymer is of biological origin, semi-synthetic or wholly synthetic. The hydrogel may be produced from a synthetic polymeric material. Such synthetic polymers can be tailored to a range of properties and predictable lot-to-lot uniformity and represent a reliable source of material that generally is free from concerns of immunogenicity. The matrices may include hydrogels formed from self-assembling peptides, such as those discussed in U.S. Pat. Nos. 5,670,483 and 5,955,343, U.S. Patent Application No. 2002/0160471, and PCT Application No. WO 02/062969.

Properties that make hydrogels valuable in drug delivery applications include the equilibrium swelling degree, sorption kinetics, solute permeability, and their in vivo performance characteristics. Permeability to compounds depends, in part, upon the swelling degree or water content and the rate of biodegradation. Since the mechanical strength of a gel may decline in proportion to the swelling degree, it is also well within the contemplation of the present invention that the hydrogel can be attached to a substrate so that the composite system enhances mechanical strength. In some embodiments, the hydrogel can be impregnated within a porous substrate, so as to gain the mechanical strength of the substrate, along with the useful delivery properties of the hydrogel.

In other embodiments, the pharmaceutical composition comprises a biocompatible matrix made of natural, modified natural or synthetic biodegradable polymers, including homopolymers, copolymers and block polymers, as well as combinations thereof.

Examples of suitable biodegradable polymers or polymer classes include any biodegradable polymers discussed within this disclosure, including but not limited to, fibrin, collagen types I, II, III, IV and V, elastin, gelatin, vitronectin, fibronectin, laminin, thrombin, poly(aminoacid), oxidized cellulose, tropoelastin, silk, ribonucleic acids, deoxyribonucleic acids; proteins, polynucleotides, gum arabic, reconstituted basement membrane matrices, starches, dextrans, alginates, hyaluron, chitin, chitosan, agarose, polysaccharides, hyaluronic acid, poly(lactic acid), poly(glycolic acid), polyethylene glycol, decellularized tissue, self-assembling peptides, polypeptides, glycosaminoglycans, their derivatives and mixtures thereof. Suitable polymers also include poly(lactide) (PLA) which can be formed of L(+) and D(−) polymers, polyhydroxybutyrate, polyurethanes, polyphoshazenes, poly(ethylene glycol)-poly(lactide-co-glycolide) co-polymer, degradable polycyanoacrylates and degradable polyurethanes. For both glycolic acid and lactic acid, an intermediate cyclic dimer is may be prepared and purified prior to polymerization. These intermediate dimers are called glycolide and lactide, respectively.

Other useful biodegradable polymers or polymer classes include, without limitation, aliphatic polyesters, poly(alkylene oxalates), tyrosine derived polycarbonates, polyiminocarbonates, polyorthoesters, polyoxaesters, polyamidoesters, polyoxaesters containing amine groups, poly(propylene fumarate), polyfumarates, polydioxanones, polycarbonates, polyoxalates, poly(alpha-hydroxyacids), poly(esters), polyurethane, poly(ester urethane), poly(ether urethane), polyanhydrides, polyacetates, polycaprolactones, poly(orthoesters), polyamino acids, polyamides and blends and copolymers thereof. Additional useful biodegradable polymers include, without limitation stereopolymers of L- and D-lactic acid, copolymers of bis(para-carboxyphenoxy)propane and sebacic acid, sebacic acid copolymers, copolymers of caprolactone, poly(lactic acid)/poly(glycolic acidypolyethyleneglycol copolymers, copolymers of polyurethane and poly(lactic acid), copolymers of alpha-amino acids, copolymers of alpha-amino acids and caproic acid, copolymers of alpha-benzyl glutamate and polyethylene glycol, copolymers of succinate and poly(glycols), polyphosphazene, poly(hydroxyalkanoates) and mixtures thereof. Binary and ternary systems also are contemplated.

In general, the material used to form a matrix is desirably configured so that it: (1) has mechanical properties that are suitable for the intended application; (2) remains sufficiently intact until tissue has in-grown and healed; (3) does not invoke an inflammatory or toxic response; (4) is metabolized in the body after fulfilling its purpose; (5) is easily processed into the desired final product to be formed; (6) demonstrates acceptable shelf-life; and (7) is easily sterilized.

In another embodiment, the population of hypoimmunogenic cells can be administered by use of a scaffold. The composition, shape, and porosity of the scaffold may be any described above. Typically, these three-dimensional biomaterials contain the living cells attached to the scaffold, dispersed within the scaffold or incorporated in an extracellular matrix entrapped in the scaffold. Once implanted into the target region of the body, these implants become integrated with the host tissue, wherein the transplanted cells gradually become established.

Non-limiting examples of scaffolds that may be used include textile structures, such as weaves, knits, braids, meshes, non-wovens, and warped knits; porous foams, semi-porous foams, perforated films or sheets, microparticles, decellularized organs or tissues, beads, and spheres and composite structures being a combination of the above structures. Nonwoven mats may, for example, be formed using fibers comprised of a synthetic absorbable copolymer of glycolic and lactic acids (PGA/PLA), sold under the tradename VICRYL sutures (Ethicon, Inc., Somerville, N.J.). Foams, composed of, for example, poly(epsilon-caprolactone)/poly(glycolic acid) (PCL/PGA) copolymer, formed by processes such as freeze-drying, or lyophilized, as discussed in U.S. Pat. No. 6,355,699, also may be utilized.

Pharmaceutical compositions may include one or more trophic factors, e.g., survival factors, growth factors, and the like, to supplement and/or further differentiate the delivered cells. In some embodiments, the one or more trophic factors is suspended within the carrier. In other embodiments, the one or more trophic factors is associated with a gel, e.g., a biocompatible and/or biodegradable polymer, such as poly(lactic acid), poly(lactic acid-co-glycolic acid), methylcellulose, hyaluronic acid, collagen, and the like, or a scaffold, as disclosed herein or as known in the art.

Pharmaceutical compositions may include preparations made from hypoimmunogenic cells that are formulated with a pharmaceutically acceptable carrier or medium. Suitable pharmaceutically acceptable carriers include any discussed within this disclosure, including but not limited to, water, salt solution (such as Ringer's solution), alcohols, oils, gelatins, polyvinyl pyrrolidine, carbohydrates such as lactose, amylose, or starch, fatty acid esters, and hydroxymethylcellulose. Such preparations can be sterilized, and if desired, mixed with auxiliary agents such as lubricants, preservatives, stabilizers, wetting agents, emulsifiers, salts for influencing osmotic pressure, buffers, and coloring agents. Pharmaceutical carriers suitable for use in the present invention are known in the art and are described, for example, in Pharmaceutical Sciences (17th Ed., Mack Pub. Co., Easton, Pa.) and WO 96/05309.

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of making hypoimmunogenic cells and cell compositions and methods of administering hypoimmunogenic cells and cell composition to a subject in need of therapy.

In addition to the above components, the subject kits can further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium on which the information has been recorded. Yet another means that may be present is a website address which may be used via the internet to access the information at a removed site. Any convenient means may be present in the kits.

### Other Embodiments of the Disclosure

Notwithstanding the appended claims, the following numbered clauses also form part of the instant disclosure.

1. A non-naturally occurring hypoimmunogenic cell comprising one or more genetic modification in the HLA-A, HLA-B and HLA-C alleles, where the modification (i) reduces or prevents a CD8+ T cell response against the cell, and (ii) reduces or prevents induction of an NK cell missing self response against the cell.

2. The non-naturally occurring hypoimmunogenic cell of clause 1, where the genetic modification is in the alpha3 domain of the HLA-A, HLA-B and HLA-C alleles.

3. The non-naturally occurring hypoimmunogenic cell of clause 2, where the genetic modification in the alpha3 domain comprises one or more genetic modifications in the HLA-A, HLA-B and HLA-C alleles selected from the group consisting of A245V, D227K, T228A, K66A and R65A.

4. The non-naturally occurring hypoimmunogenic cell of clause 3, where the genetic modification in the alpha3 domain further comprises a mutation in the HLA-E, HLA-F and/or HLA-G alleles.

5. The non-naturally occurring hypoimmunogenic cell of clause 1, where the genetic modification comprises one or more introduced in-frame stop codon mutations.

6. The non-naturally occurring hypoimmunogenic cell of clause 5, where the introduced in-frame stop codon mutation is in the alpha1 domain, alpha2 domain, alpha3 domain, transmembrane domain, intracellular domain and/or signal peptide region of the HLA-A, HLA-B and HLA-C alleles.

7. The non-naturally occurring hypoimmunogenic cell any one of clauses 1-6, where the genetic modification is introduced using a gene editing technique.

8. The non-naturally occurring hypoimmunogenic cell of clause 7, where the gene editing technique is selected from the group consisting of prime editing and base editing.

9. The non-naturally occurring hypoimmunogenic cell of any one of clauses 1-8, where the cell is a human cell.

10. The non-naturally occurring hypoimmunogenic cell of any one of clauses 1-8, where the cell is an immune cell.

11. The non-naturally occurring hypoimmunogenic cell of any one any one of clauses 1-8, where the cell is selected from the group consisting of a T cell, a B cell, an NK cell and a dendritic cell.

12. The non-naturally occurring hypoimmunogenic cell of any one of clauses 1-8, where the cell is an induced pluripotent stem cell (iPSC).

13. The non-naturally occurring hypoimmunogenic cell of any one of clauses 1-8, where the cell is a somatic cell differentiated or derived from an iPSC.

14. The non-naturally occurring hypoimmunogenic cell of any one 1-13, where the cell further comprises at least one heterologous transgene.

15. The non-naturally occurring hypoimmunogenic cell of any one clauses 1-13, where the heterologous transgene encodes a chimeric antigen receptor.

16. A method of preparing a hypoimmunogenic cell, the method comprising a step of introducing into a cell a genetic modification in the HLA-A, HLA-B and HLA-C alleles, where the modification (i) reduces or prevents a CD8+ T cell response against the cell, and (ii) reduces or prevents induction of an NK cell missing self response against the cell.

17. The method of clause 16, where the genetic modification is in the alpha3 domain of the HLA-A, HLA-B and HLA-C alleles.

18. The method of clause 17, where the genetic modification in the alpha3 domain comprises a mutation in the HLA-A, HLA-B and HLA-C alleles selected from the group consisting of A245V, D227K, T228A, K66A and R65A.

19. The method of clause 18, where the genetic modification in the alpha3 domain further comprises a mutation in the HLA-E, HLA-F and/or HLA-G alleles.

20. The method of clause 16, where the genetic modification comprises an introduced in-frame stop codon mutation.

21. The method of clause 20, where the introduced in-frame stop codon mutation is in the alpha1, alpha1 or alpha3 domain of the HLA-A, HLA-B and HLA-C alleles.

22. The method of any one of clauses 16-21, where the genetic modification is introduced using a gene editing technique.

23. The method clause 22, where the gene editing technique is selected from the group consisting of prime editing and base editing.

24. The method of clause 23, where the genetic modification is an A245V mutation introduced using a small guide RNA comprising a sequence selected from the group consisting of GCGGCUGUGGUGGUGCCUUC (SEQ ID NO: 39) and GCAGCUGUGGUGGUGCCUUC (SEQ ID NO: 40).

25. The method of clause 23, where the genetic modification is an in-frame stop codon mutation introduced using a small guide RNA comprising a sequence selected from the group consisting of CCAGAAGUGGGCGGCUGUGG (SEQ ID NO: 41), AGCAGGAGGGGCCGGAGUAU (SEQ ID NO: 42) and GCAGGACGCCUACGACGGCA (SEQ ID NO: 43).

26. The method of any one of clauses 20, 21 and 25, further comprising the step of introducing a heterologous transgene encoding HLA-E or a transgene encoding a mutant HLA-A/B/C expressing A245V, D227K and T228A single mutations or any combination thereof.

27. The method of any one of clauses 16-26, where the cell is a human cell.

28. The method of any one of clauses 16-26, where the cell is an immune cell.

29. The method of any one of clauses 16-26, where the cell is selected from the group consisting of a T cell, a B cell, an NK cell and a dendritic cell.

30. The method of any one of clauses 16-26, where the cell is an induced pluripotent stem cell (iPSC).

31. The method of any one of clauses 16-26, where the cell is a somatic cell differentiated or derived from an iPSC.

32. The method of any one of clauses 16-31, further comprising a step of introducing at least one heterologous transgene.

33. The method of any one of clauses 16-31, further comprising a step of introducing at least one heterologous transgene encoding a chimeric antigen receptor.

34. A pharmaceutical composition comprising (i) hypoimmunogenic cells according to any one of clauses 1-15 or (ii) hypoimmunogenic cells prepared according to the method of any one of clauses 16-33, and a physiologically acceptable excipient.

35. A method of treating a disease or condition comprising administering to a subject in need thereof a pharmaceutical composition according to 34.

36. The method according to clause 35, wherein the disease or condition is selected from the group consisting of cancer, myocardial infarction, blindness, spinal cord injury, ALS, Parkinson's disease, Huntington's disease, Alzheimer's disease, enteric neuropathy, multiple sclerosis, osteoarthritis, skin disease, diabetes, liver disease, osteoporosis, DiGeorge syndrome, kidney disease and an immune disorder.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1: Targeted A245V Mutation of MHC-I Alleles to Disrupt Allo-Reactivity

The following sgRNAs were designed to induce the A245V mutation of MHC-I alleles HLA-A, HLA-B, HLA-C, HLA-E, HLA-G by a cytosine base editor, thereby preventing the binding of the CD8 coreceptor to MHC-I and subsequently disrupting allo-reactive TCR binding to the allogeneic cellular product.

Exemplary small guide RNA (sgRNA) sequences containing the gene specific crRNA are shown below:

(SEQ ID NO. 39)
GCGGCUGUGGUGGUGCCUUC;
(SEQ ID NO. 40)
GCAGCUGUGGUGGUGCCUUC.

Exemplary sequences when expressed as a full sgRNA are shown below:

(SEQ ID NO. 62)
mG*mC*mG* rGrCrU rGrUrG rGrUrG rGrUrG rCrCrU rUrCrG rUrUrU rUrArG rArGrC
rUrArGrArArA rUrArG rCrArA rGrUrU rArArA rArUrA rArGrG rCrUrA rGrUrC rCrGrU
rUrArU rCrArA rCrUrU rGrArA rArArA rGrUrG rGrCrA rCrCrG rArGrU rCrGrG rUrGrC
mU*mU*mU* rU;
(SEQ ID NO. 63)
mG*mC*mA* rGrCrU rGrUrG rGrUrG rGrUrG rCrCrU rUrCrG rUrUrU rUrArG rArGrC
rUrArGrArArA rUrArG rCrArA rGrUrU rArArA rArUrA rArGrG rCrUrA rGrUrC rCrGrU
rUrArU rCrArA rCrUrU rGrArA rArArA rGrUrG rGrCrA rCrCrG rArGrU rCrGrG rUrGrC
mU*mU*mU* rU.

“*”=phosphorothioate linkage
“m”=2-O-Methyl modification
“r” is used preceding an RNA base

Transient delivery via electroporation of the base editor and sgRNA(s) as mRNAs and/or RNPs generates a MHC-I mutation and mitigates T cell dependent allogenic rejection.

This mutated MHC-I prevents binding of recipient CD8 to donor MHC-I, preventing T cell allogenic rejection through the mechanism of non-self T cell killing. Targeted mutations of MHC-I also prevent host NK cell killing of allografted NK cells via maintaining allograft NK “self” signaling to the host NK cells. Therefore, MHC-I mutation protects allografted cells from host immune cell recognition and subsequent rejection by T-cells and NK cells through a ‘non-self’ response and ‘missing self’ response, respectively.

Successful editing of the MHC-I is determined by sequencing, flow cytometry and SNP q-PCR.

To assess resistance of the edited hypo-immune NK cells to alloreactive T cell killing, alloreactive recipient T cells are expanded and purified. Edited donor NK cells and T cells are co-cultured together at various Effector (T cells): Target (NK cell) ratios. T cell dependent allogenic killing of the edited NK cells is monitored via flow cytometry over the time course of 24 hrs. Recipient T cells are also dyed with CFSE and alloreactive T cell expansion is determined by flow cytometry.

To determine whether the edited NK cells are resistant to loss of self NK killing, different ratios of Effector (allo NK cells): Target (edited NK cells) are prepared and monitored for three to five days by flow cytometry.

### Example 2: Targeted Introduction of In-Frame Stop Codon Mutation of MHC-1 Alleles to Disrupt Allo-Reactivity

The following sgRNAs were designed to induce an in-frame stop codon mutation in the MHC-I alleles of HLA-A, HLA-B, HLA-C, HLA-E, HLA-G by a cytosine base editor, thereby preventing the expression of MHC-I on the cell surface and allowing for the simplified re-expression of an HLA-E transgene. Transient delivery of a base editor and sgRNA(s) as mRNAs and/or RNPs to mediate MHC-I knock-out will reduce the potential for unwanted off-target effects. This also allows for the re-introduction/expression of specific HLA alleles/mutants without the need for sequence alterations to confer resistance to knock-out guide RNAs. Introducing these changes into allograft cells will protect them from host immune cell recognition and rejection by T-cells and NK cells through a ‘non-self’ response and ‘missing self’ response, respectively.

Exemplary small guide RNA (sgRNA) sequence containing the gene specific crRNA are shown below:

(SEQ ID NO. 41)
CCAGAAGUGGGCGGCUGUGG;
(SEQ ID NO. 42)
AGCAGGAGGGGCCGGAGUAU;
(SEQ ID NO. 43)
GCAGGACGCCUACGACGGCA.

Exemplary sequences when expressed as a full sgRNA are shown below:

(SEQ ID NO. 64)
mC*mC*mA*rGrArArGrUrGrGrGrCrGrGrCrUrGrUrGrGrGrUrUrUrUrArGrArGrCrUrArGrAr
ArArUrArGrCrArArGrUrUrArArArArUrArArGrGrCrUrArGrUrCrCrGrUrUrArUrCrArArCrUr
UrGrArArArArArGrUrGrGrCrArCrCrGrArGrUrCrGrGrUrGrCmU*mU*mU*rU;
(SEQ ID NO. 65)
mA*mG*mC*ArGrGrArGrGrGrGrCrCrGrGrArGrUrArUrGrUrUrUrUrArGrArGrCr
UrArGrArArArUrArGrCrArArGrUrUrArArArArUrArArGrGrCrUrArGrUrCrCrGrUrUrArUrCr
ArArCrUrUrGrArArArArArGrUrGrGrCrArCrCrGrArGrUrCrGrGrUrGrCmU*mU*mU*rU;
(SEQ ID NO. 66)
mG*mC*mA*GrGrArCrGrCrCrUrArCrGrArCrGrGrCrArGrUrUrUrUrArGrArGrCr
UrArGrArArArUrArGrCrArArGrUrUrArArArArUrArArGrGrCrUrArGrUrCrCrGrUrUrArUrCr
ArArCrUrUrGrArArArArArGrUrGrGrCrArCrCrGrArGrUrCrGrGrUrGrCmU*mU*mU*rU.

Additionally, illustrative in-frame stop guides specific for a cytosine base editor (e.g. BE3, BE4) are shown below:

(SEQ ID NO. 50)
UACCGGCAGGACGCCUACGA;
(SEQ ID NO. 51)
GGAGCAGCGGAGAGUCUACC;
(SEQ ID NO. 52)
CGCUGCAGCGCACGGGUACC;
(SEQ ID NO. 53)
GACCUGGCAGCGGGAUGGGG;
(SEQ ID NO. 54)
GGAGGACCAGACCCAGGACA;
(SEQ ID NO. 55)
GACCCAGGACACGGAGCUCG;
(SEQ ID NO. 56)
CACACCAUCCAGAUAAUGUA.

### Example 3: Targeted Introduction of Double Mutant HLA D227K/T228A into the MHC-I Alleles to Disrupt Allo-Reactivity

The following pegRNA was designed to induce the D227K/T228A mutation of MHC-I alleles HLA-A, HLA-B, HLA-C, HLA-E, HLA-G by prime editor 2, thereby preventing the binding of the CD8 coreceptor to MHC-I and subsequently disrupting allo-reactive TCR binding to our cellular product. Although T-cell receptor interaction is disrupted, NK cell recognition is retained, preventing NK-mediated ‘missing self’ rejection without the need to engineer cells to express exogenous protein construct(s).

### Example pegRNA Sequence

(SEQ ID NO. 67)
mG*mG*mA*rUrGrGrGrGrArGrGrArCrCrArGrArCrCrCrGrUrUrUrUrArGrArGrCrUrArGrAr
ArArUrArGrCrArArGrUrUrArArArArUrArArGrGrCrUrArGrUrCrCrGrUrUrArUrCrArArCrUr
UrGrArArArArArGrUrGrGrCrArCrCrGrArGrUrCrGrGrUrGrCrGrCrUrCrCrGrCrCrUrUrCrUr
GrGrGrUrCrUrGrGrUrC*mC*mU*mC.

Disruption of allo-reactivity is assessed using a MLR-based assay. Donor/stimulator PBMC or WT tumor cells are irradiated (30Gy) and incubated with alloreactive recipient PBMC at a ratio of 1:1 or 2:1. Recipient/responder PBMC cells are re-challenged with irradiated donor PBMC or WT tumor cells for 21 days. Expansion and re-challenge experiments are performed in a GREX using RPMI+15% HAB serum+Sodium Pyruvate+NEAA+beta-mercaptoethanol+GlutaMAX+HEPES++IL-2+IL-7+IL-15 as the expansion media. After 21 days, expanded alloreactive cells are purified using CD4 and CD8 magnetic bead selection and validated for purity by FACS (anti-CD3, anti-CD4 and anti-CD8 antibodies). Purified T cells are used immediately or cryo-preserved in liquid nitrogen prior to alloreactive killing assays.

Purified alloreactive T cells (effector) are incubated with T cells or NK cells or tumor cells (NALM-6-GFP) for control and hypo-immune experimental conditions; stimulator T cells and NK cells are derived from the original donor cells (Target). Autologous controls are included where T cells from the same donor are incubated together with the relevant experimental conditions. Target cells are stained with amine binding cell trace dye. Effector to Target ratios are incubated at 1:1, 5:1 and 15:1 and incubated for 24 hrs at 37° C., 5% CO₂. Target cell death is evaluated by FACS. Effector cell intra-cellular IFN-gamma and TNF-alpha are evaluated by FACS.

Additional MLR assays are performed using responder PBMC cells, stained with CFSE for T cell division, incubated with control and experimental engineered tumor cells (NALM-6-GFP and Jurkat cells). Tumor Jurkat cells are cell trace violet tagged. Experimental conditions include mitomycin C treated conditions and untreated conditions. T cells expansion by CFSE, IFN-gamma expression, and TNF-alpha expression was analyzed by FACS analysis at 4, 5, 6, or 7 days. Reduced cell division, decreased IFN-gamma expression and/or decreased TNF-alpha expression indicate hypo-immune engineered cells.

Resistance of the engineered cells to NK killing specific to “loss of self” is evaluated in cNK and activated NK killing assays. Assays are performed in RPMI+10% FBS+P/S+IL-15+IL-7. cNK and activated NK cells (effector cells) are incubated with engineered control and experimental cells (Target cells) at an E:T ratio of 1:1, 3:1 and 9:1. For IncuCyte NK killing assays, target cells are identified using amine binding cell trace dyes, GFP tagged cells or nucRed cells. Assay are incubated at 37° C., 5% CO₂, and wells are imaged for target cell signal every 2 hrs for 3-5 days. Target cell counts over the analysis period are normalized to the baseline (time 0 hr). For cNK and activated NK assays, technical replicates are n=5-6. Assays are repeated using effector cells from n=2 or n=3 donors. NK specific killing of control and hypo-immune engineered experimental cell conditions is determined by changes in target cell numbers, normalized to base-line, over the analysis time period. NK cell purity and expression of NKG2A are evaluated by FACS using anti-CD3 plus anti-CD56 and anti-NKG2A antibodies, respectively.

### Example 4: Efficient Gene Editing of MHC-I Genes Using Cytosine and Adenine Base Editors to Disrupt Gene Splicing and Protein Production

Experiments were performed to assess the efficiency of multiple methods of base editing in targeting the different MHC-I alleles, including HLA-A, HLA-B, HLA-C, HLA-H, HLA-K, and HLA-L. The following sgRNAs were designed to induce the A245V mutation of the MHC-I alleles by a cytosine base editor (BE4) or a adenine base editor (ABE8e).

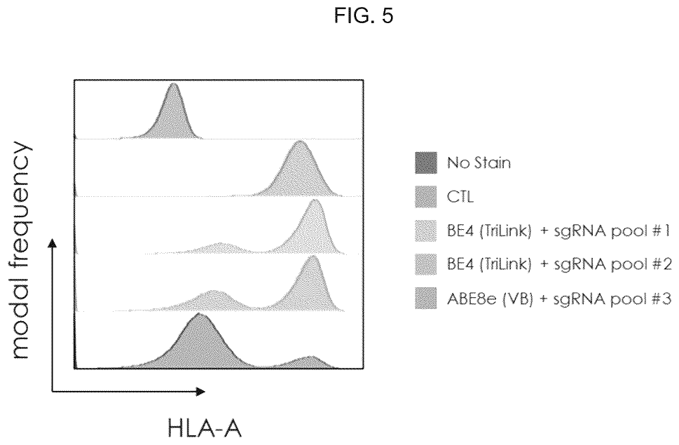

Exemplary small guide RNA (sgRNA) sequences containing the gene specific crRNA, used as sgRNA pools #1, #2, and #3 in FIG. 5, are shown below:

sgRNA pool #1 [With a cytosine base editor BE4]
(SEQ ID NO. 50)
UACCGGCAGGACGCCUACGA;
(SEQ ID NO. 51)
GGAGCAGCGGAGAGUCUACC;
(SEQ ID NO. 52)
CGCUGCAGCGCACGGGUACC;
(SEQ ID NO. 53)
GACCUGGCAGCGGGAUGGGG;
(SEQ ID NO. 57)
CCUUACCCCAUCUCAGGGUG [Splice donor exon 6 (SDe6)].
sgRNA pool #2 [With a cytosine base editor BE4]
(SEQ ID NO. 58)
CGAGCCAGAAGAUGGAGCCG [Stop1];
(SEQ ID NO. 59)
UUACCCCAUCUCAGGGUGAG [Stop2];
(SEQ ID NO. 60)
UGACGGCCAUCCUCGGCGUC [Start].
sgRNA pool #3 [With an adenine base editor ABE8e]
(SEQ ID NO. 61)
CUACGUAGGGUCCUUCAUCC [Splice acceptor exon 3
(SAe3)];
(SEQ ID NO. 57)
CCUUACCCCAUCUCAGGGUG [SDe6];
(SEQ ID NO. 60)
UGACGGCCAUCCUCGGCGUC [Start]

Exemplary sequences when expressed as a full sgRNA are shown below:

Full sgRNA 0034
(SEQ ID NO. 68)
mU*mA*mC*rCrGrGrCrArGrGrArCrGrCrCrUrArCrGrA rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU
Full sgRNA 0035
(SEQ ID NO. 69)
mG*mG*mA*rGrCrArGrCrGrGrArGrArGrUrCrUrArCrC rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU
Full sgRNA 0036
(SEQ ID NO. 70)
mC*mG*mC*rUrGrCrArGrCrGrCrArCrGrGrGrUrArCrC rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU
Full sgRNA 0037
(SEQ ID NO. 71)
mG*mA*mC*rCrUrGrGrCrArGrCrGrGrGrArUrGrGrGrG rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU
Full sgRNA SDe6
(SEQ ID NO. 72)
mC*mC*mU*rUrArCrCrCrCrArUrCrUrCrArGrGrGrUrG rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU
Full sgRNA Stop1
(SEQ ID NO. 73)
mC*mG*mA*rGrCrCrArGrArArGrArUrGrGrArGrCrCrG rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU
Full sgRNA Stop2
(SEQ ID NO. 74)
mU*mU*mA*rCrCrCrCrArUrCrUrCrArGrGrGrUrGrArG rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU
Full sgRNA Start
(SEQ ID NO. 75)
mU*mG*mA*rCrGrGrCrCrArUrCrCrUrCrGrGrCrGrUrC rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU
Full sgRNA SAe3
(SEQ ID NO. 76)
mC*mU*mA*rCrGrUrArGrGrGrUrCrCrUrUrCrArUrCrC rG rUrUrU rUrArG rArGrC rUrArG
rArArA rUrArG rCrArA rGrUrU rArArA rArUrA rArGrG rCrUrA rGrUrC rCrGrU rUrArU
rCrArA rCrUrU rGrArA rArArA rGrUrG rGrCrA rCrCrG rArGrU rCrGrG rUrGrC
mU*mU*mU* rU

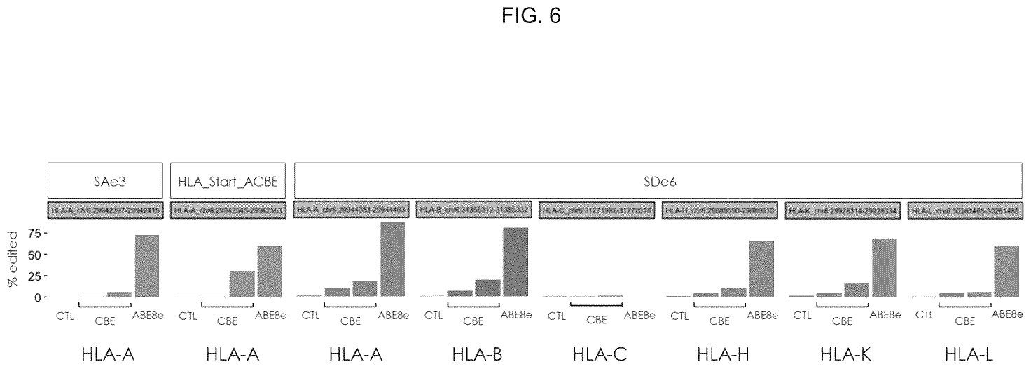

NK cells were isolated from cryopreserved Leukopaks using a CD56 negative selection kit and electroporated with either BE4 mRNA (CBE) or ABE8e mRNA and and the indicated sgRNAs pools. Electroporation was performed using a Maxcyte ATx electroporator, employing a pre-installed program from Maxcyte, Expanded T cell 4. Cells were allowed to recover and expand for 4 days post-electroporation. Successful editing of the MHC-I alleles was determined by sequencing, flow cytometry, and SNP q-PCR. As shown in FIG. 5, flow cytometry analysis was performed to quantify HLA-A expression in the edited cells. A significant percentage of the NK cells edited with the cytosine base editor BE4 expressed HLA-A levels comparable to control (CTL), unedited NK cells. Only a small percentage of these edited NK cells had reduced HLA-A expression. Conversely, NK cell editing with the adenine base editor ABE8e led to reduced HLA-A expression in a high percentage of the NK cells. The percent of cells that were edited at the target site at the indicated HLA loci was quantified by a Targeted Sequencing Panel and NGS (next-generation sequencing). As shown in FIG. 6, use of ABE8e consistently led to 50%-80% of cells being edited at the target loci, and use of cytosine base editors (CBE) led to at most 30% of the cells being edited. These results show that the MHC-I genes were successfully targeted and edited by base editing and that ABE outperformed CBE in efficiency when targeting multiple loci.

### Example 5: Engineered Hypoimmune NALM6 Cells Avoid Allogeneic T and NK Cell-Meditated Cytotoxicity

Experiments were performed to demonstrate the ability of engineered hypoimmune cells to avoid host-mediated rejection. NALM6 cells, an immortalized B cell line, were used as a model system. These cells were engineered to express MHC-I proteins that bind to inhibitory receptors on NK cells but do not interact with CD8 on the T cell surface. Accordingly, these engineered cells can avoid both CD8+ T cell-mediated cytotoxicity due to MHC mismatch and NK cell-mediated cytotoxicity triggered by lack of cell surface MHC-I expression.

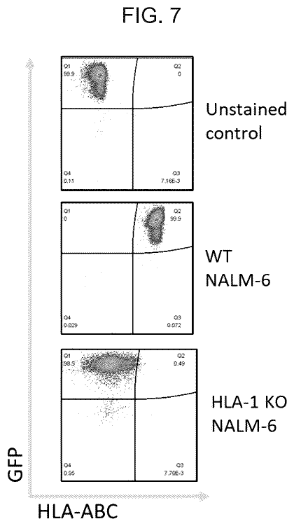

GFP+NALM6 cells were engineered as follows. MHC class I genes were knocked out using CRISPR-Cas9 and a guide RNA which targets exon 4 of HLA Class I molecules (HLA-1): CCAGAAGUGGGCGGCUGUGG (SEQ ID NO: 77). Following electroporation of the CRISPR-Cas9 and sgRNA to delete HLA-1, cells were allowed to recover and divide. The cells were then subjected to magnetic separation to purify the HLA-1 knockout cells. Briefly, the cells were labeled with an APC-tagged anti-HLA-ABC antibody, mixed with anti-APC microbeads, and incubated together. The labeled, HLA positive cells were then depleted from the population using the AutoMacs system. As demonstrated by FIG. 7, flow cytometry analysis showed while WT NALM-6 cells expressed HLA-A, HLA-B, and HLA-C on the cell surface, the expression of these proteins was not detected in HLA-1 knockout NALM6 cells after CRISPR-Cas9-mediated gene editing.

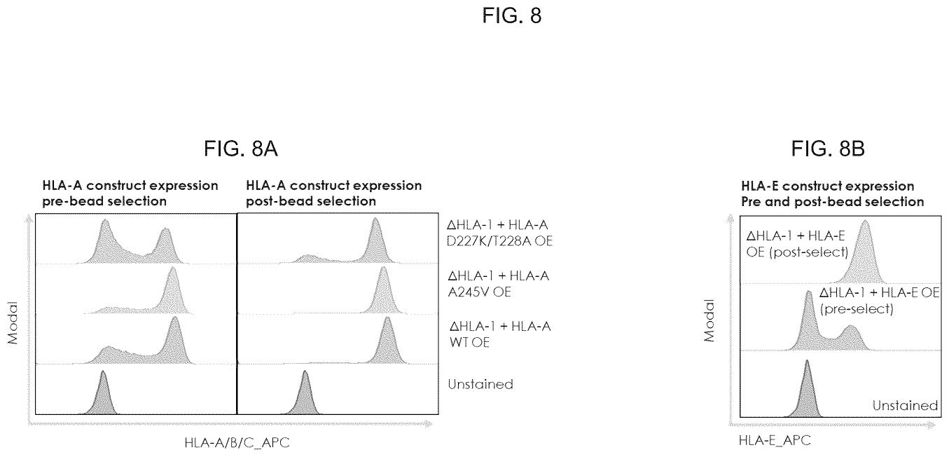

WT HLA-A (HLAA*03:01), mutant HLA-A-A245V, double mutant HLA-A-D227K/T228A, and HLA-E were tested for their ability to protect NALM6 cells from conventional NK cell (cNK) killing. Each molecule was overexpressed in the HLA-1 knockout cells using lentiviral vectors. HLA-1 knockout cells overexpressing WT HLA-A (ΔHLA-1+HLA-A WT OE), mutant HLA-A-A245V (ΔHLA-1+HLA-A A245V OE), double mutant HLA-A-D227K/T228A (ΔHLA-1+HLA-A D227K/T228A OE) (as shown in FIG. 8A), or HLA-E (ΔHLA-1+HLA-E OE) (as shown in FIG. 8B) were subsequently enriched using magnetic bead selection.

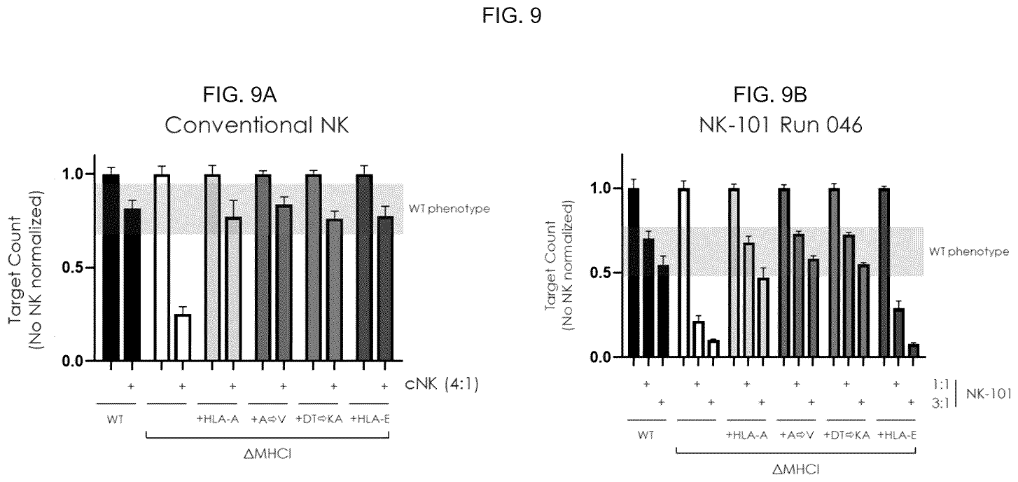

To assess the ability of the above HLA proteins to protect cells from NK cell killing, NALM6 ΔHLA-1 (NALM6-ΔMHCI) cells were transduced with lentiviral vectors to express the HLA proteins and then assayed for survival relative to WT NALM6 cells. As shown in FIG. 9, NALM6 target cell survival when challenged with conventional NK (cNK) cells was analyzed by Incucyte after coculturing at an E:T of 4:1 for 8 days. NALM6 target cell survival (relative) when challenged with memory NK cells was analyzed by Incucyte after coculturing at an E:T of 1:1 or 3:1 for 7 days (n=6; bars are relative cell count+SD normalized to Target only). As shown in FIG. 9A, WT NALM6 cells were relatively resistant to conventional NK cell (cNK) killing but became susceptible to killing after the HLA-1 genes are knocked out (ΔMHCI). Importantly, overexpression of WT HLA-A, mutant HLA-A-A245V, double mutant HLA-A-D227K/T228A, and HLA-E restored protection from cNK killing to the WT phenotype relative to NALM6-ΔMHCI cells. As shown in FIG. 9B, overexpression of WT HLA-A, mutant HLA-A-A245V, and double mutant HLA-A-D227K/T228A also protected NALM6-ΔMHCI cells from killing by memory NK cells, which have enhanced tumor cytotoxicity. However, HLA-E overexpression did not confer any appreciable protection from memory NK killing relative to ΔMHCI cells. These results suggest that allogeneic cells relying on HLA-E for protection will likely be rejected by memory NK cells.

To assess the ability of the above HLA proteins to protect allogeneic cells from T cell killing, NALM6 ΔHLA-1 (NALM6-ΔMHCI) cells overexpressing WT HLA-A, mutant HLA-A-A245V, double mutant HLA-A-D227K/T228A, and WT HLA-E were cocultured with peripheral blood mononuclear cells (PBMCs).

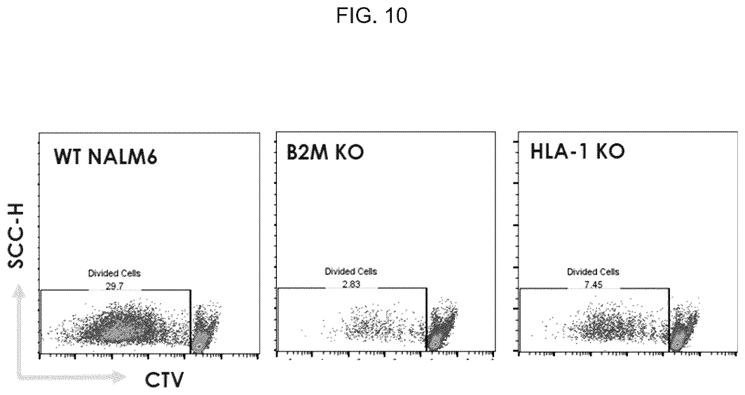

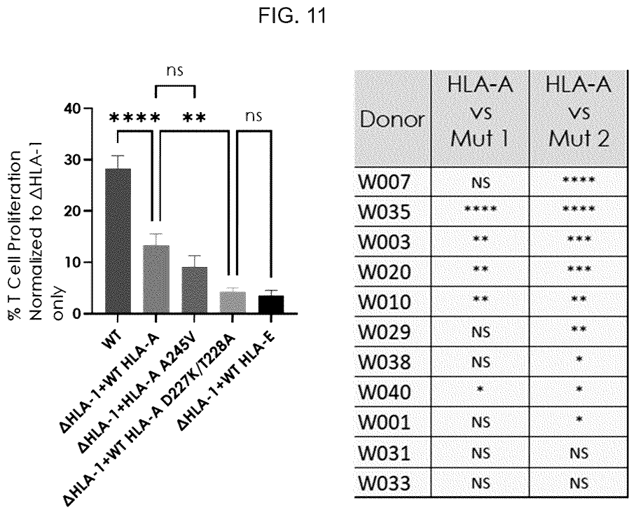

First, CD8+ T cell proliferation was measured by flow cytometry. As shown in FIG. 10, WT NALM6 cells induced T cell proliferation, demonstrated by the dilution of the CellTrace Violet (CTV) fluorescent stains in the divided T cells. However, similar to NALM6 cells with β2m knockout (B2M KO), NALM6 cells with HLA Class I knockout (HLA-1 KO) failed to activate T cells. CD8+ T cell proliferation induced by NALM6 cells with genetically modified HLA genes was calculated by subtraction of background T cell proliferation as determined by the percent T cell proliferation induced by HLA-1 KO only controls (normalized to HLA-1 KO only). Importantly, as shown in FIG. 11, the percent of CD8+ T cell proliferation from 11 donors demonstrated that ΔHLA-1+WT HLA-A and ΔHLA-1+WT HLA-A A245V mutant NALM6 cells led to reduced T cell proliferation compared to WT NALM6 cells. Moreover, ΔHLA-1+WT HLA-A D227K/T228A mutant NALM6 cells prevented T cell alloreactivity and led to an even lower percent of T cell proliferation, similar to the low percent of T cell proliferation induced by ΔHLA-1+WT HLA-E NALM6 cells.

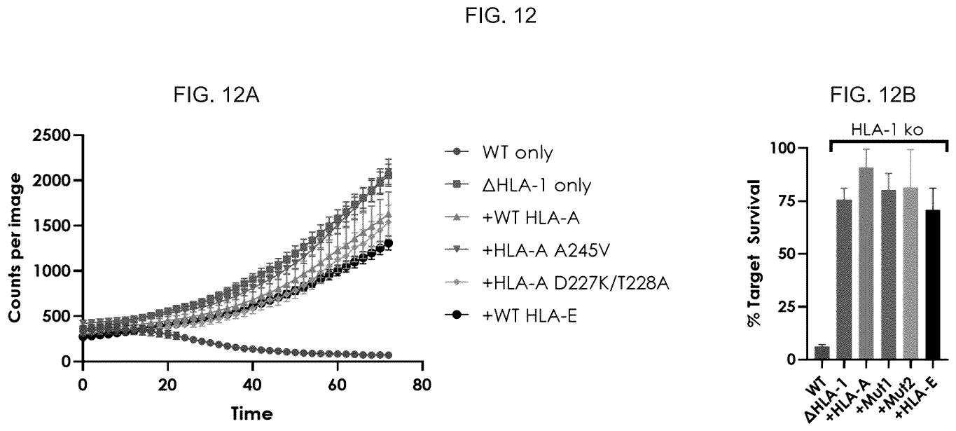

T cell cytotoxicity was then assessed by GFP+ target cell counts as measured and analyzed by Incucyte. Notably, as shown in FIG. 12A, target counts of NALM6 ΔHLA-1 (ΔHLA-1 only) cells overexpressing WT HLA-A, mutant HLA-A-A245V, double mutant HLA-A-D227K/T228A, and WT HLA-E were similar to that of NALM6 ΔHLA-1 cells and significantly higher compared to WT NALM6 cells (WT only) after 72 hours of coculturing. Furthermore, percent target survival was measured by flow cytometry at 48 hours. As shown in FIG. 12B, while only about 5% of WT NALM6 cells were alive, 70-85% of NALM6 ΔHLA-1 cells and NALM6 ΔHLA-1 cells overexpressing WT HLA-A (+HLA-A), mutant HLA-A-A245V (+Mut1), double mutant HLA-A-D227K/T228A (+Mut2), and WT HLA-E (+HLA-E) remained alive. Together, these results show that overexpression of WT HLA-A, mutant HLA-A-A245V, double mutant HLA-A-D227K/T228A, and WT HLA-E did not restore T cell killing of NALM6 ΔHLA-1 cells.

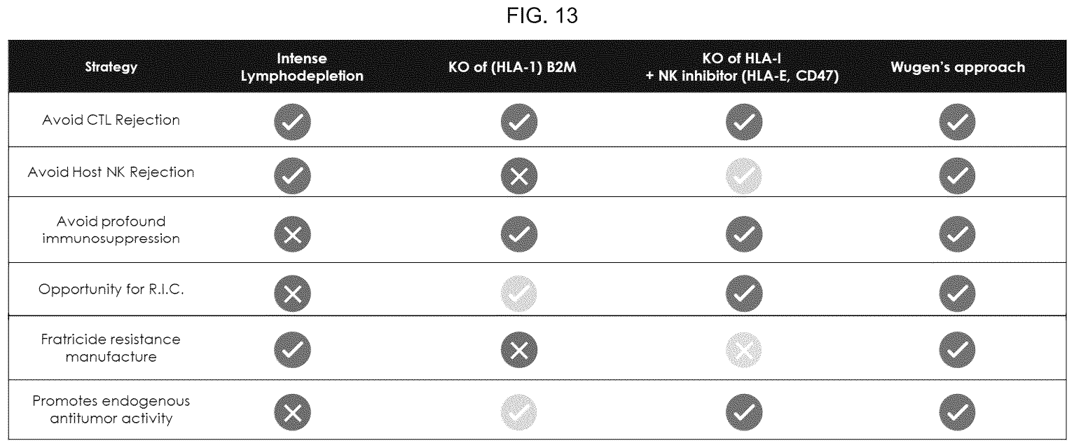

In conclusion, engineered hypoimmune cells expressing mutant HLA-I proteins avoided CD8+ T cell-mediated cytotoxicity and NK cell-mediated cytotoxicity because they lack cell surface MHC-I expression. These distinguishing features make the engineered cells of the present disclosure superior to other allogeneic cell products. As summarized in FIG. 13, the engineered cells (1) avoid CTL and NK cell rejection, (2) do not require profound immunosuppression, (3) provide an opportunity for reduced intensity conditioning, (4) are not susceptible to fratricide during manufacturing, and (5) promote endogenous antitumor activity.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, embodiments, and embodiments of the invention as well as specific examples thereof, are intended to encompass both structural and functional equivalents thereof.

Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. An hypoimmunogenic cell comprising one or more genetic modifications in one or more HLA genes selected from the group consisting of: HLA-A, HLA-B, HLA-C, HLA-E, HLA-F, and HLA-G, wherein the modification (i) reduces or prevents a CD8+ T cell-mediated response against the hypoimmunogenic cell, and/or (ii) reduces or prevents an NK cell-mediated response against the hypoimmunogenic cell.

2. The hypoimmunogenic cell of claim 1, wherein the one or more genetic modifications comprises a modification in one or more domains of the one or more HLA genes selected from the group consisting of: alpha 1 domain, alpha 2 domain, alpha 3 domain, transmembrane domain, intracellular domain, and signal peptide region.

3. The hypoimmunogenic cell of claim 1, wherein the one or more genetic modifications comprises a modification in the alpha 3 domain of the one or more HLA genes.

4. The hypoimmunogenic cell of claim 1, wherein the one or more genetic modifications encode one or more single amino acid substitutions.

5. The hypoimmunogenic cell of claim 2, wherein the one or more genetic modifications encode one or more modifications at one or more amino acid residues selected from the group consisting of: A245, D227, T228, K66, and R65.

6. The hypoimmunogenic cell of claim 2, wherein the one or more genetic modifications encode one or more amino acid substitutions selected from the group consisting of: A245V, D227K, T228A, K66A, and R65A.

7. The hypoimmunogenic cell of claim 1, wherein the one or more genetic modifications encode a modification at A245 in HLA-A, HLA-B, and HLA-C.

8. The hypoimmunogenic cell of claim 7, wherein the modification is A245V.

9. The hypoimmunogenic cell of claim 1, wherein the one or more genetic modifications encode a modification at D227 in HLA-A, HLA-B, and HLA-C and a modification at T228 in HLA-A, HLA-B, and HLA-C.

10. The hypoimmunogenic cell of claim 9, wherein the modification at D227 is D227K and the modification at T228 is T228A.

11. The hypoimmunogenic cell of claim 1, wherein the one or more genetic modifications comprises one or more in-frame stop codon mutations.

12. The hypoimmunogenic cell of claim 11, wherein the hypoimmunogenic cell comprises one or more exogenously expressed HLA genes, optionally wherein the HLA genes comprise one or more mutations.

13. The hypoimmunogenic cell of claim 12, wherein the hypoimmunogenic cell comprises an exogenously expressed HLA-E gene.

14. The hypoimmunogenic cell of claim 1, wherein the genetic modification is introduced using a gene editing technique, optionally wherein the gene editing technique is selected from the group consisting of CRISPR-Cas9 gene editing, prime editing, and base editing.

15. The hypoimmunogenic cell of claim 1, wherein the hypoimmunogenic cell is differentiated or derived from a stem cell, optionally wherein the stem cell is an induced pluripotent stem cell (iPSC).

16. The hypoimmunogenic cell of claim 1, wherein the hypoimmunogenic cell is an immune cell.

17. The hypoimmunogenic cell of claim 16, wherein the hypoimmunogenic cell is selected from the group consisting of: a T cell, a B cell, an NK cell, a dendritic cell, and a macrophage.

18. The hypoimmunogenic cell of claim 1, wherein the hypoimmunogenic cell further comprises a polynucleotide encoding a cell surface receptor, optionally wherein the cell surface receptor is a chimeric receptor.

19. A method of making a hypoimmunogenic cell, the method comprising introducing into a cell one or more genetic modifications in one or more HLA genes selected from the group consisting of: HLA-A, HLA-B, HLA-C, HLA-E, HLA-F, and HLA-G; wherein the modification (i) reduces or prevents a CD8+ T cell-mediated response against the hypoimmunogenic cell, and/or (ii) reduces or prevents an NK cell-mediated response against the hypoimmunogenic cell.

20.-40. (canceled)

41. A pharmaceutical composition comprising the hypoimmunogenic cell of claim 1.

42. A method of treating a disease or condition in a subject in need thereof, comprising administering to the subject the pharmaceutical composition of claim 41.

43.-44. (canceled)
